(19) United States
(12) Patent Application Publication
(10) Pub. No.: US 2022/0204540 A1
(43) Pub. Date: Jun. 30, 2022

(54) ESTERS OF NON- AROMATIC HETEROCYCLIC COMPOUNDS HAVING A NEMATOCIDAL ACTIVITY, THEIR AGRONOMIC COMPOSITIONS AND USE THEREOF

(71) Applicant: ISAGRO S.p.A., Milan (IT)

(72) Inventors: Marilena GUSMEROLI, Monza (MB) (IT); Giuseppe D'ORAZIO, Bollate (MI) (IT); Entela SINANI, Novara (IT); Daniele FORGIA, Borgomanero (NO) (IT); Paolo BELLANDI, Carcare (SV) (IT); Chiara SARGIOTTO, Galliate (NO) (IT); Daniele BIANCHI, Bellinzago Novarese (NO) (IT); Jessica VILLATA, Cameri (NO) (IT); Riccardo LIGUORI, Monza (MB) (IT)

(21) Appl. No.: 17/604,892

(22) PCT Filed: Apr. 28, 2020
(86) PCT No.: PCT/IB2020/053992

§ 371 Date: Oct. 19, 2021

(30) Foreign Application Priority Data
Apr. 30, 2019 (IT) 102019000006460

(51) Int. Cl. C07F 9/6574; C07F 9/40; C07D 207/16; C07D 211/60; C07D 211/78; C07D 233/02; C07D 285/12; C07D 279/06; C07D 277/14; C07D 277/06; C07D 277/56; C07D 263/06; C07D 263/24; C07D 263/34; C07D 263/26; A01N 57/24; A01N 47/16; A01N 43/76; A01N 43/86; A01N 43/50; A01N 43/82; A01N 43/78; A01N 43/40; A01N 43/36

(52) U.S. Cl. CPC C07F 9/65742; A01N 43/36; C07D 207/16; C07D 211/60; C07D 211/78; C07D 233/02; C07D 285/12; C07D 279/06; C07D 277/14; C07D 277/06; C07D 277/56; C07D 263/06; C07D 263/24; C07D 263/34; C07D 263/26; A01N 57/24; A01N 47/16; A01N 43/76; A01N 43/86; A01N 43/50; A01N 43/82; A01N 43/78; A01N 43/40; C07F 9/4018

(57) ABSTRACT

Non-aromatic fluoroalkenyl heterocyclic compounds having general formula (I), agronomic compositions containing said compounds having formula (I) and their use for the control of nematodes in agricultural crops, are described.

The present invention relates to new non-aromatic fluoroalkenyl heterocyclic compounds.

The present invention also relates to agronomic compositions which contain said compounds having formula (I) and their use for the control of nematodes in agricultural crops.

### STATE OF THE ART

Esters of fluoroalkenyl heterocyclic compounds have been described in literature for use as pesticides and, in particular, as nematocides.

These compounds consist of three characteristic residues: an aromatic heterocycle, a residue —(CH₂)ₙ—COO— wherein n is an integer ranging from 0 to 2 and a fluoroalkenyl chain having general formula —(CH₂)ₘ—CX═CF₂ wherein m can vary from 1 to 6 and X is a hydrogen atom or a halogen.

Patent application JP2000/038379 describes pyridine ester compounds substituted in position 2, 3 or 4 with a fluoroalkenyl chain and suitable substituents on the ring.

Patent application JP2000/086636 describes pyrazole derivatives bearing the above-mentioned fluoroalkenyl chain.

Other heterocyclic groups such as thiophen-2-yl, furan-2-yl, pyrazin-2-yl, quinol-4-yl or pyrrol-2-yl are described in patent application JP2000/186073, substituted with the same fluoroalkenyl chain.

All of these compounds, however, have proved to be unsatisfactory in terms of nematocidal activity, as they cannot effectively limit the attack of the parasite and reduce the formation of galls on the root system of the plant.

Furthermore, in various cases, these products are phytotoxic with respect to important agricultural crops at the doses that allow a good nematocidal activity to be obtained, demonstrating a significant necrosis of the leaves and the stem.

### DESCRIPTION

The Applicant has now surprisingly found new esters of fluoroalkenyl heterocyclic compounds that overcome the drawbacks indicated above, being characterized by a high nematocidal activity even at low doses and which, at the same time, are well tolerated by agricultural crops.

The present invention relates to non-aromatic heterocyclic compounds with 5 or 6 terms substituted in position 2 with a fluoroalkenyl chain, having general formula (I):

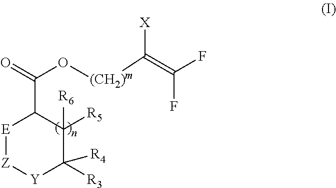

wherein:
E represents an oxygen atom, a sulfur atom or an N-A group;
Y represents a sulfur atom possibly oxidized to an S(O) or S(O)₂ group, an oxygen atom, an NR₇ group or a CH₂ group;
A represents a hydrogen atom, a C₁-C₆-alkyl group, a C₁-C₆-haloalkyl group, a C₂-C₇ alkenyl group, a C₂-C₇ haloalkenyl group, a C₃-C₆-cycloalkyl group, a C₃-C₆-cycloalkylalkylC₁-C₆ group, a C₁-C₆-alkylsulfinyl group, a C₁-C₆-alkylsulfonyl group, a C₁-C₆-haloalkylsulfonyl group, an arylsulfonyl group, a C₁-C₆-alkanoyl group, a formyl group, a C₁-C₆-haloalkanoyl group, a C₃-C₆-cycloalkanoyl group, an aroyl group, a heterocyclylcarbonyl group, a C₁-C₆-thioalkanoyl group, a C₁-C₆-thiohaloalkanoyl group, a C₃-C₆-thiocycloalkanoyl group, a thioaroyl group, a thioheterocyclylcarbonyl group, a C₁-C₆-alkoxycarbonyl group, a C₁-C₆-haloalkoxycarbonyl group, a C₃-C₆ cycloalkoxycarbonyl group, an aryloxycarbonyl group, a benzyloxycarbonyl group, a heterocyclyloxycarbonyl group, a C₁-C₆-alkylthiocarbonyl group, a C₁-C₆-haloalkylthiocarbonyl group, a C₃-C₆ cycloalkylthiocarbonyl group, an arylthiocarbonyl group, a benzoylthiocarbonyl group, a heterocyclylthiocarbonyl group, a C₁-C₆-alkylaminocarbonyl group, a C₁-C₆-dialkylaminocarbonyl group, a C₁-C₆-haloalkylaminocarbonyl group, a C₁-C₆-dihaloalkylaminocarbonyl group, a C₃-C₆ cycloalkylaminocarbonyl group, a C₁-C₆-dicycloalkylaminocarbonyl group, an arylaminocarbonyl group, a heterocyclylaminocarbonyl group, a C₁-C₆-alkyl-carbonylalkyl-C₁-C₆ group, a C₁-C₆-haloalkyl-carbonylalkyl-C₁-C₆ group, a C₃-C₆ cycloalkylcarbonylalkyl-C₁-C₆ group, an arylcarbonylalkyl-C₁-C₆ group, a benzylcarbonylalkyl-C₁-C₆ group, a heterocyclycarbonylalkyl-C₁-C₆ group, a C₁-C₆-alkyl-carbonyloxyalkyl-C₁-C₆ group, a C₁-C₆-alkyl-carbonyloxyhaloalkyl-C₁-C₆ group, a C₁-C₆-alkyl-carbonyloxycycloalkyl-C₃-C₆ group, a C₁-C₆-alkyl-carbonyloxyaryl group, a C₁-C₆-alkyl-carbonyloxybenzyl group, a C₁-C₆-alkyl-carbonyloxyheterocyclic group; a C₁-C₆-alkyl-carbonylthioalkyl-C₁-C₆ group, a C₁-C₆-alkyl-carbonylthiohaloalkyl-C₁-C₆ group; a C₁-C₆-alkyl-carbonylthiocycloalkyl-C₃-C₆ group, a C₁-C₆-alkyl-carbonylthioaryl group, a C₁-C₆-alkyl-carbonylthiobenzyl group, a C₁-C₆-alkyl-carbonylthioheterocyclic group, a C₁-C₆-alkyl-aminocarbonylalkyl-C₁-C₆ group, a C₁-C₆-haloalkyl-aminocarbonylalkyl-C₁-C₆ group, a C₁-C₆-dialkyl-aminocarbonylalkyl-C₁-C₆ group, a C₁-C₆-dihaloalkyl-aminocarbonylalkyl-C₁-C₆ group, a C₃-C₆-cycloalkylamino-carbonyl-alkyl-C₁-C₆ group, a C₆-C₁₂-dicycloalkyl-aminocarbonylalkyl-C₁-C₆ group, an aryl-aminocarbonylalkyl-C₁-C₆ group, a benzyl-aminocarbonylalkyl-C₁-C₆ group, a heterocyclyl-aminocarbonylalkyl-C₁-C₆ group, a C₁-C₆-alkanoyloxyalkyl-C₁-C₆ group, a C₁-C₆-haloalkanoyloxyalkyl-C₁-C₆ group, a C₃-C₆-cycloalkanoyloxyalkyl-C₁-C₆ group, an aroyloxyalkyl-C₁-C₆ group, a benzoyloxyalkyl-C₁-C₆ group, a heterocyclylcarbonyloxyalkyl-C₁-C₆ group, a C₁-C₆-alkoxyalkyl-C₁-C₆ group, a C₁-C₆-haloalkoxyalkyl-C₁-C₆ group, a C₃-C₆-cycloalkoxyalkyl-C₁-C₆ group, an aryloxyalkyl-C₁-C₆ group, a benzyloxyalkyl-C₁-C₆ group, a heterocyclyloxyalkyl-C₁-C₆ group, a C₁-C₆-alkanoylthioalkyl-C₁-C₆ group, a C₁-C₆-haloalkanoylthioalkyl-C₁-C₆ group, a C₃-C₆-cycloalkanoylthioalkyl-C₁-C₆ group, an aroylthioalkyl-C₁-C₆ group, a benzoylthioalkyl-C₁-C₆ group, a heterocyclylcarbonylthioalkyl-C₁-C₆ group, a C₁-C₆-alkylthioalkyl-C₁-C₆ group, a C₁-C₆-haloalkylthioalkyl-C₁-C₆ group, a C₃-C₆-cycloalkylthioalkyl-C₁-C₆ group, an arylthioalkyl-C₁-C₆ group, a benzylthioalkyl-C₁-C₆ group, a heterocyclylthioalkyl-C₁-C₆ group, a C₁-C₆-alkanoylaminoalkyl-C₁-C₆ group, a C₁-C₆-haloalkanoylaminoalkyl-C₁-C₆ group, a C₃-C₆-cycloalkanoylaminoalkyl-C₁-C₆ group, an aroylaminoalkyl-C₁-C₆ group, a benzoylaminoalkyl-C₁-C₆ group, a heterocyclylcarbonylaminoalkyl-C₁-C₆ group, a C₁-C₆-dialkylphosphoryl group, a diarylphosphoryl group, a C₁-C₆-dialkylthiophosphoryl group, a diarylthiophosphoryl group, a 2-oxa-1,3,2-dioxaphosphorinan-2-yl group possibly substituted by one or more C₁-C₆ alkyl groups, a 2-oxa-1,3,2-dioxaphospholan-2-yl group possibly substituted by one or more C₁-C₆ alkyl groups;
Z represents a C═O bond, a C═S bond or a R₁—C—R₂ group

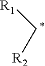

n represents an integer ranging from 0 to 1;
m represents an integer ranging from 1 to 6;
X represents a hydrogen or fluorine atom;
R₁ and R₂ the same or different represent a hydrogen atom, a halogen atom selected from fluorine, chlorine, bromine or iodine, a C₁-C₆ alkyl group, a C₁-C₆ haloalkyl group, a C₂-C₇ alkenyl group, a C₂-C₇ haloalkenyl group, a C₃-C₆-cycloalkyl group, an aryl group, a heterocyclic group, a C₃-C₆-cycloalkylalkyl-C₁-C₆ group, a C₁-C₆-alkoxycarbonyl group, a C₁-C₆-haloalkoxycarbonyl group, a C₃-C₆ cycloalkoxycarbonyl group, an aryloxycarbonyl group, a heterocyclyloxycarbonyl group, a C₁-C₆-alkylthiocarbonyl group, a C₁-C₆-haloalkylthiocarbonyl group, a C₃-C₆ cycloalkylthiocarbonyl group, an arylthiocarbonyl group, a heterocyclylthiocarbonyl group, a C₁-C₆-alkylaminocarbonyl group, a C₁-C₆-dialkylaminocarbonyl group, a C₁-C₆-haloalkylaminocarbonyl group, a C₁-C₆-dihaloalkylaminocarbonyl group, a C₃-C₆ cycloalkylaminocarbonyl group, a C₃-C₆ dicycloalkylaminocarbonyl group, an arylaminocarbonyl group, a heterocyclylaminocarbonyl group, a nitrile, a C₁-C₆-alkoxyl group, a C₁-C₆-thioalkoxyl group, a C₁-C₆-haloalkoxyl group, a C₃-C₆-cycloalkoxyl group, a C₁-C₆-halothioalkoxyl group, a C₃-C₆-cyclothioalkoxyl group, a C₁-C₆-alkanoyl group, a C₁-C₆-haloalkanoyl group, a C₃-C₆ cycloalkanoyl group, an aroyl group, a heterocyclylcarbonyl group, a glucosyl group, galactosyl, mannosyl, saccharosyl, lactosyl, maltosyl, arabisonyl, xylosyl, ribosyl, aminoglucosyl, N-acetylaminoglucosyl wherein the OH groups present on said sugars can be optionally substituted to form OR₇ groups;
or R₁ and R₂ together with the carbon atom to which they are bound form a C₃-C₆ cycloalkyl ring;
R₇ represents a C₁-C₆ alkyl group, a benzyl group, a C₁-C₆-alkanoyl group;
R₃, R₄, R₅ and R₆ the same or different represent a hydrogen atom, a halogen atom selected from fluorine, chlorine, bromine or iodine, a C₁-C₆ alkyl group, a C₁-C₆ haloalkyl group or a C₃-C₆ cycloalkyl group, an aryl group, a benzyl group, a heterocyclic group;
excluding the compound wherein:
E=N-A;
A=benzyloxycarbonyl;
Z═Y═CH₂;
R₃═R₄═H;
n=0;
m=2;
X═F

In the present description, when indicating a numerical range, the extremes are also meant to be included in the same.

A C₁-C₆-alkyl-carbonylalkyl-C₁-C₆ group, C₁-C₆-haloalkyl-carbonylalkyl-C₁-C₆ group, C₃-C₆-cycloalkylcarbonylalkyl-C₁-C₆ group, arylcarbonylalkyl-C₁-C₆ group, benzylcarbonylalkyl-C₁-C₆ group, heterocyclylcarbonylalkyl-C₁-C₆ group, refers to a radical having formula RaC(═O)Rb wherein Ra respectively has the meanings of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are propylcarbonylethyl, phenylcarbonylethyl, isopropylcarbonylbutyl.

A C₁-C₆-alkyl-carbonyloxyalkyl-C₁-C₆ group; C₁-C₆-alkyl-carbonyloxyhaloalkyl-C₁-C₆ group; C₁-C₆-alkyl-carbonyloxycycloalkyl-C₃-C₆ group; C₁-C₆-alkyl-carbonyloxyaryl group, C₁-C₆-alkyl-carbonyloxybenzyl group, C₁-C₆-alkyl-carbonyloxyheterocyclic group, refers to a radical having formula RbC(═O)ORa wherein Ra respectively has the meanings of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are propylcarbonyloxyethyl, phenylcarbonyloxypropyl, isopropylcarbonyloxybutyl.

A C₁-C₆-alkyl-carbonylthioalkyl-C₁-C₆ group, C₁-C₆-alkyl-carbonyl-thiohaloalkyl-C₁-C₆ group; C₁-C₆-alkyl-carbonylthiocycloalkyl-C₃-C₆ group, C₁-C₆-alkyl-carbonylthioaryl group, C₁-C₆-alkyl-carbonylthiobenzyl group, C₁-C₆-alkyl-carbonylthioheterocyclic group, refers to a radical having formula RbC(═O)SRa wherein Ra has the meanings of C₁-C₆ alkyl, C₁-C₆ haloalkyl, C₃-C₆ cycloalkyl, aryl, benzyl and heterocyclyl respectively and Rb has the meaning of C₁-C₆ alkyl.

Examples of said groups are propylcarbonylthioethyl, phenylcarbonylthiopropyl, isopropylcarbonylthiopentyl.

A C₁-C₆-alkyl-aminocarbonylalkyl-C₁-C₆ group, C₁-C₆-haloalkyl-aminocarbonylalkyl-C₁-C₆ group, C₁-C₆-dialkyl-aminocarbonylalkyl-C₁-C₆ group, C₁-C₆-dihaloalkyl-aminocarbonylalkyl-C₁-C₆ group, C₃-C₆-cycloalkyl-aminocarbonylalkyl-C₁-C₆ group, C₆-C₁₂-dicycloalkyl-aminocarbonylalkyl-C₁-C₆ group, aryl-aminocarbonylalkyl-C₁-C₆ group, benzylaminocarbonylalkyl-C₁-C₆ group, heterocyclyl-aminocarbonylalkyl-C₁-C₆ group refers to a radical having formula RaNHC(═O)Rb or formula (Ra)₂NC(═O)Rb wherein Ra respectively has has the meanings of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are propylaminocarbonylethyl, phenylaminocarbonylpropyl, isopropylaminocarbonylbutyl.

A C₁-C₆ alkanoyloxyalkyl C₁-C₆ group, C₁-C₆ haloalkanoyloxyalkyl C₁-C₆ group, C₄-C₁₈ cycloalkanoyloxyalkyl C₁-C₁₂ group, aroyloxyalkyl C₁-C₁₂ group, C₁-C₁₂ benzoyloxyalkyl group, C₁-C₁₂ heterocyclylcarbonyloxyalkyl group, refers to a radical having formula RaC(═O)ORb wherein Ra respectively has the meanings of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are propanoyloxymethyl, cyclohexanoyloxymethyl, benzoyloxyethyl.

C₂-C₁₂ alkanoylthioalkyl C₁-C₁₂, C₂-C₁₂ haloalkanoylthioalkyl C₁-C₁₂, C₄-C₁₈ cycloalkanoylthioalkyl C₁-C₁₂, aroylthioalkyl C₁-C₁₂, benzoylthioalkyl C₁-C₁₂, heterocyclylcarbonylthioalkyl, refer to a radical having formula RaC(═O)SRb wherein Ra respectively has the meanings of C₁-C₁₂ alkyl, C₁-C₁₂ haloalkyl, C₃-C₁₈ cycloalkyl, aryl, benzyl and heterocyclyl and Rb has the meaning of C₁-C₁₂ alkyl.

Examples of said groups are propanoylthiomethyl, cyclohexanoyl-thiomethyl, benzoylthioethyl.

C₁-C₆-alkoxyalkyl-C₁-C₆, C₁-C₆-haloalkoxyalkyl-C₁-C₆, C₃-C₆-cycloalkoxyalkyl-C₁-C₁₂, aryloxyalkyl-C₁-C₆, benzyloxyalkyl-C₁-C₆, heterocyclyloxyalkyl C₁-C₆, refer to a radical having formula RaORb wherein Ra has the meaning of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are ethoxymethyl, trifluoromethoxymethyl, phenoxyethyl.

C₁-C₆-alkylthioalkyl-C₁-C₆, C₁-C₆ haloalkylthioalkyl-C₁-C₆, C₃-C₆ cycloalkylthioalkyl-C₁-C₆, arylthioalkyl C₁-C₆, benzylthioalkyl C₁-C₆, heterocyclylthioalkyl C₁-C₆ refer to a radical having formula RaSRb wherein Ra has the meaning of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are ethylthiomethyl, cyclopropylthiomethyl, phenylthioethyl.

C₁-C₆ alkanoylaminoalkyl-C₁-C₁₂, C₁-C₆ haloalkanoylaminoalkyl-C₁-C₆, C₃-C₆ cycloalkanoylaminoalkyl-C₁-C₆, C₁-C₆ aroylamino-alkyl, C₁-C₆ benzoylamino-alkyl, C₁-C₆ heterocyclylcarbonylaminoalkyl, refer to a radical having formula RaC(═O)NHRb wherein Ra has the meaning of C₁-C₆ alkyl, C₁-C₆ haloalkyl, C₃-C₆ cycloalkyl, aryl, benzyl and heterocyclyl and Rb has the meaning of C₁-C₆ alkyl.

Examples of said groups are propanoylaminomethyl, cyclohexanoylaminomethyl, benzoylaminoethyl.

A C₁-C₆-alkanoyl group, C₁-C₆-haloalkanoyl group, C₃-C₆-cycloalkanoyl group, aroyl group, heterocyclylcarbonyl group, refers to a radical having formula RaC(═O)— wherein Ra has the meaning of C₁-C₆ alkyl, C₁-C₆ haloalkyl, C₃-C₆ cycloalkyl, aryl and heterocyclyl.

Examples of said groups are acetyl, trifluoroacetyl, 2,4-difluorobenzoyl, cyclopropylcarbonyl.

A C₁-C₆-thioalkanoyl group, C₁-C₆-thiohaloalkanoyl group, C₃-C₆-thiocycloalkanoyl group, thioaroyl group, thioheterocyclycylcarbonyl group, refers to a radical having formula RaC(═S)— wherein Ra has the meaning of C₁-C₆ alkyl, C₁-C₆ haloalkyl, C₃-C₆ cycloalkyl, aryl and heterocyclyl.

Examples of said groups are thioacetyl, 2,4-difluorothiobenzoyl, cyclopropylthiocarbonyl.

A C₁-C₆-alkoxycarbonyl group, C₁-C₆-haloalkoxycarbonyl group, C₃-C₆-cycloalkoxycarbonyl group, aryloxycarbonyl group, benzyloxycarbonyl group, heterocyclyloxycarbonyl group, refers to a radical having formula RaOC(═O)— wherein Ra has the meaning of C₁-C₆ alkyl, C₁-C₆ haloalkyl, C₃-C₆ cycloalkyl, aryl, benzyl and heterocyclyl.

Examples of said groups are t-butoxycarbonyl, benzyloxycarbonyl, methoxycarbonyl, cyclopentoxycarbonyl.

A C₁-C₆-alkylthiocarbonyl group, C₁-C₆-haloalkylthiocarbonyl group, C₃-C₆-cycloalkylthiocarbonyl group, arylthiocarbonyl group, benzylthiocarbonyl group, heterocyclycylthiocarbonyl group, refers to a radical having formula RaSC(═O)— wherein Ra has the meaning of C₁-C₆ alkyl, C₁-C₆ haloalkyl, C₃-C₆ cycloalkyl, aryl and heterocyclyl.

Examples of said groups are t-butylthiocarbonyl, benzylthiocarbonyl, ethylthiocarbonyl, cyclopropylthiocarbonyl.

Examples of halogen are fluorine, chlorine, bromine, iodine.

A C₁-C₆-alkyl group, refers to a linear or branched C₁-C₆ alkyl radical, optionally substituted by aryl, heterocyclic, cycloalkyl, alkoxycarbonyl, cycloalkoxycarboxyl, alkoxyl, phenoxyl, cycloalkoxyl, thioalkoxyl, N-alkyl aminocarbonyl, N,N-dialkylaminocarbonyl, nitrile, acyl and benzoyl groups.

Examples of C₁-C₆ alkyl are methyl, ethyl, n-propyl, isopropyl, n-butyl, isobutyl, sec-butyl, tert-butyl, n-pentyl, 3-methylbutyl, n-hexyl, 3,3-dimethylbutyl.

Examples of C₁-C₁₂ halo alkyl are fluoromethyl, difluoromethyl, trifluoromethyl, chloromethyl, dichloromethyl, 2,2,2-trifluoroethyl, 1,1,2,2-tetrafluoroethyl, pentafluoroethyl, heptafluoropropyl, 4,4,4-trichloro-butyl, 4,4-difluoropentyl, 5,5-difluorohexyl.

A C₃-C₆-cycloalkyl group refers to a carbocyclic grouping containing from three to six carbon atoms possibly substituted by alkyl, haloalkyl, alkenyl, haloalkenyl, aryl, heterocyclic, cycloalkyl, halogen, alkoxycarbonyl, cycloalkoxycarbonyl, alkoxyl, cycloalkoxyl, thioalkoxyl, phenoxyl, N-alkylaminocarbonyl, N,N-dialkylaminocarbonyl, CN, acyl and benzoyl groups.

Examples of C₃-C₆-cycloalkyl are cyclopropyl, cyclobutyl, cyclopentyl, cyclohexyl.

Examples of C₂-C₇ alkenyl are: ethenyl, propenyl, butenyl.

Examples of C₂-C₇ haloalkenyls are: 2,2-dichloro-propenyl, 1,2,2-trichloropropenyl.

Examples of C₃-C₆-cycloalkylalkyl-C₁-C₆ are: 2-ethylcycyclopropyl, cyclopentylmethyl, 3-propylhexyl.

Heterocyclic groups refer to cyclic systems with 5 or 6 terms, aromatic or non-aromatic, possibly benzocondensed or heterobicyclic, containing at least one heteroatom chosen from nitrogen, oxygen, sulfur.

Examples of heterocycles are: thiazole, 1,3,4 thiadiazole, pyrrolidine, piperidine, morpholine, pyrazole etc.

Examples of aryls, wherein aryl refers to mono, bi or tricyclic aromatic systems, composed of carbon atoms alone, are phenyl, naphthyl, phenanthrenyl, anthracenyl.

All aryl, benzyl, phenoxyl and heterocyclic systems can be substituted by one or more groups selected from halogens, C₁-C₆-alkyl, C₁-C₆-haloalkyl, C₃-C₆-cycloalkyl, C₃-C₆-cycloalkylalkyl-C₁-C₆, C₁-C₆-alkoxyl, C₁-C₆-haloalkoxyl, C₁-C₆-thioalkoxyl, C₁-C₆-thiohaloalkoxyl, C₁-C₆-alkylsulfinyl, C₁-C₆-alkylsulfonyl, C₁-C₆-alkoxycarbonyl, C₃-C₆-cycloalkoxycarbonyl, amino, N—C₁-C₆-alkylamino, C₁-C₆-dialkylamino, C₃-C₆-cycloalkylamino, C₆-C₁₂-dicycloalkylamino, C₁-C₆-alkylaminocarbonyl, C₃-C₆-cycloalkylaminocarbonyl, C₁-C₆-alkylcarbonyl, carboxyl, cyano, aryl, benzyl.

The following are also intended to fall within the spirit of the present invention:
a) all possible geometric isomers of the compounds having general formula (I) deriving from particular meanings of the substituents R₁—R₇, Z, Y and A
b) salts of the compounds having formula (I) obtained by the addition of inorganic or organic acids;
c) any hydrated forms of the compounds having formula (I).

Examples of preferred compounds having general formula (I), are compounds wherein E, Z, Y, R₃, R₄, R₅, R₆, X, n and m have the meanings indicated in Table 1:

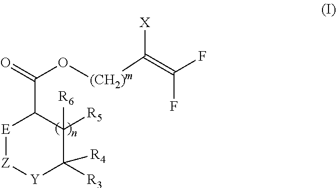

TABLE 1

| Comp | | | | | | | | | | |
|---|---|---|---|---|---|---|---|---|---|---|
| Nr. | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO(Cypropyl) | CH₂ | S | F | H | H | — | — | 2 | 0 |
| 7 | N—COCH₃ | C—(CH₃)₂ | S | F | CH₃ | CH₃ | — | — | 2 | 0 |
| 8 | N—COCH₃ | CH₃—C—COOCH₃ | S | F | H | H | — | — | 2 | 0 |
| 9 | N—COCH₃ | CH- | S | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 10 | N—COCH₃ | CH-(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH₃)₂ | CH₂ | S | F | H | H | — | — | 2 | 0 |
| 13 | N—CO(Cypentyl) | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 16 | N—COCH₃ | CF₃—C—CH₃ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 21 | N—COSCH(CH₃)₂ | CH₂ | S | F | H | H | — | — | 2 | 0 |
| 22 | N—COCH₃ | CH₂ | S | F | H | H | H | H | 2 | 1 |
| 23 | N—COCH₃ | CH₂ | S | F | H | H | — | — | 2 | 0 |
| 24 | N—COCH₂CH₃ | CH₂ | S | F | H | H | H | H | 2 | 1 |
| 25 | N—COCH₃ | CH₂ | S(O) | F | H | H | — | — | 2 | 0 |
| 26 | N—CH₂COCH₃ | CH₂ | S | F | H | H | — | — | 2 | 0 |
| 27 | N—CH₂COCypentyl | CH₂ | S | F | H | H | — | — | 2 | 0 |
| 28 | N—CH₂CO-(4-F)-Ph | CH₂ | S | F | H | H | — | — | 2 | 0 |
| 29 | N—CH₂CO-2- | CH₂ | S | F | H | H | — | — | 2 | 0 |
| | thiazolyl | | | | | | | | | |
| 30 | N—CH₂ | CH₂ | S | F | H | H | — | — | 2 | 0 |
| | CH₂CH₂COOCH₃ | | | | | | | | | |
| 31 | N—CH₂CH₂COO- | CH₂ | S | F | H | H | — | — | 2 | 0 |
| | (Cypropyl) | | | | | | | | | |
| 32 | N—CH₂CH₂COOCH₂Ph | CH₂ | S | F | H | H | — | — | 2 | 0 |
| 33 | N—CH₂CH₂COSCH₃ | CH₂ | S | F | H | H | — | — | 2 | 0 |
| 34 | N—CH₂CH₂CONHCH₃ | CH₂ | S | F | H | H | — | — | 2 | 0 |
| 35 | N—CH₂OCOCH₃ | CH₂ | S | F | H | H | — | — | 2 | 0 |
| 36 | N—CH₂OCOCypentyl | CH₂ | S | F | H | H | — | — | 2 | 0 |
| 37 | N—CH₂OCObenzyl | CH₂ | S | F | H | H | — | — | 2 | 0 |
| 38 | N—CH₂OCH(CH₃)₂ | CH₂ | S | F | H | H | — | — | 2 | 0 |
| 39 | N—CH₂Obenzyl | CH₂ | S | F | H | H | — | — | 2 | 0 |
| 40 | N—CH₂SCH(CH₃)₂ | CH₂ | S | F | H | H | — | — | 2 | 0 |
| 41 | N—CH₂SCOCH₃ | CH₂ | S | F | H | H | — | — | 2 | 0 |
| 42 | N—CH₂SCOCypentyl | CH₂ | S | F | H | H | — | — | 2 | 0 |
| 43 | N—COCH₃ | CH(CH₃)₂ | S(O) | F | H | H | — | — | 2 | 0 |
| 44 | N—COCH₃ | CH(CH₃)₂ | S(O)₂ | F | H | H | — | — | 2 | 0 |
| 45 | N—CH₂COCH₃ | CH(CH₃)₂ | S | F | H | H | — | — | 2 | 0 |
| 46 | N—CH₂COCypentyl | CH(CH₃)₂ | S | F | H | H | — | — | 2 | 0 |
| 47 | N—CH₂CO-(4-F)-Ph | CH(CH₃)₂ | S | F | H | H | — | — | 2 | 0 |
| 48 | N—CH₂CO-₂-Thiazolyl | CH(CH₃)₂ | S | F | H | H | — | — | 2 | 0 |
| 49 | N—CH₂CH₂COOCH₃ | CH(CH₃)₂ | S | F | H | H | — | — | 2 | 0 |
| 50 | N—CH₂CH₂COO | CH(CH₃)₂ | S | F | H | H | — | — | 2 | 0 |
| | Cypropyl | | | | | | | | | |
| 51 | N—CH₂CH₂COOCH₂Ph | CH(CH₃)₂ | S | F | H | H | — | — | 2 | 0 |
| 52 | N—CH₂CH₂COSCH₃ | CH(CH₃)₂ | S | F | H | H | — | — | 2 | 0 |
| 53 | N—CH₂CH₂CONHCH₃ | CH(CH₃)₂ | S | F | H | H | — | — | 2 | 0 |
| 54 | N—CH₂OCOCH₃ | CH(CH₃)₂ | S | F | H | H | — | — | 2 | 0 |
| 55 | N—CH₂OCOCypentyl | CH(CH₃)₂ | S | F | H | H | — | — | 2 | 0 |
| 56 | N—CH₂OCOBenzyl | CH(CH₃)₂ | S | F | H | H | — | — | 2 | 0 |
| 57 | N—CH₂OCH(CH₃)₂ | CH(CH₃)₂ | S | F | H | H | — | — | 2 | 0 |
| 58 | N—CH₂Obenzyl | CH(CH₃)₂ | S | F | H | H | — | — | 2 | 0 |
| 59 | N—CH₂SCH(CH₃)₂ | CH(CH₃)₂ | S | F | H | H | — | — | 2 | 0 |
| 60 | N—CH₂SCOCH₃ | CH(CH₃)₂ | S | F | H | H | — | — | 2 | 0 |
| 61 | N—CH₂SCOCypentyl | CH(CH₃)₂ | S | F | H | H | — | — | 2 | 0 |
| 62 | N—CH₂CO-(2,4-diCl)-Ph | CF₃—C—CH₃ | O | F | H | H | — | — | 2 | 0 |
| 63 | N—COButyl | CF₃—C—CH₃ | O | F | H | H | — | — | 2 | 0 |
| 64 | N—CH₂COCH₃ | CF₃—C—CH₃ | O | F | H | H | — | — | 2 | 0 |
| 65 | N—CH₂COCypentyl | CF₃—C—CH₃ | O | F | H | H | — | — | 2 | 0 |
| 66 | N—CH₂CO-(4-F)-Ph | CF₃—C—CH₃ | O | F | H | H | — | — | 2 | 0 |
| 67 | N—CH₂CO-2- | CF₃—C—CH₃ | O | F | H | H | — | — | 2 | 0 |
| | Thiazolyl | | | | | | | | | |
| 68 | N—CH₂CH₂COOCH₃ | CF₃—C—CH₃ | O | F | H | H | — | — | 2 | 0 |
| 69 | N—CH₂CH₂COO- | CF₃—C—CH₃ | O | F | H | H | — | — | 2 | 0 |
| | (Cypropyl) | | | | | | | | | |
| 70 | N—CH₂CH₂COOCH₂Ph | CF₃—C—CH₃ | O | F | H | H | — | — | 2 | 0 |
| 71 | N—CH₂CH₂COSCH₃ | CF₃—C—CH₃ | O | F | H | H | — | — | 2 | 0 |
| 72 | N—CH₂CH₂CONHCH₃ | CF₃—C—CH₃ | O | F | H | H | — | — | 2 | 0 |
| 73 | N—CH₂OCOCH₃ | CF₃—C—CH₃ | O | F | H | H | — | — | 2 | 0 |
| 74 | N—CH₂OCO(Cypentyl) | CF₃—C—CH₃ | O | F | H | H | — | — | 2 | 0 |
| 75 | N—CH₂OCOBenzyl | CF₃—C—CH₃ | O | F | H | H | — | — | 2 | 0 |
| 76 | N—CH₂OCH(CH₃)₂ | CF₃—C—CH₃ | O | F | H | H | — | — | 2 | 0 |
| 77 | N—CH₂OBenzyl | CF₃—C—CH₃ | O | F | H | H | — | — | 2 | 0 |
| 78 | N—CH₂SCH(CH₃)₂ | CF₃—C—CH₃ | O | F | H | H | — | — | 2 | 0 |
| 79 | N—CH₂SCOCH₃ | CF₃—C—CH₃ | O | F | H | H | — | — | 2 | 0 |
| 80 | N—CH₂SCOCypentyl | CF₃—C—CH₃ | S | F | H | H | — | — | 2 | 0 |
| 81 | N—COCH₃ | CH₂ | NH | F | H | H | — | — | 2 | 0 |
| 82 | N—CH₂CO- | CF₃—C—CH₃ | NH | F | H | H | — | — | 2 | 0 |
| | (2,4-diCl)-Ph | | | | | | | | | |
| 83 | N—COButyl | CF₃—C—CH₃ | NH | F | H | H | — | — | 2 | 0 |
| 84 | N—CH₂COCH₃ | CF₃—C—CH₃ | NH | F | H | H | — | — | 2 | 0 |
| 85 | N—CH₂COCypentyl | CF₃—C—CH₃ | NH | F | H | H | — | — | 2 | 0 |
| 86 | N—CH₂CO-(4-F)-Ph | CF₃—C—CH₃ | NH | F | H | H | — | — | 2 | 0 |
| 87 | N—CH₂CO-2- | CF₃—C—CH₃ | NH | F | H | H | — | — | 2 | 0 |
| | Thiazolyl | | | | | | | | | |
| 88 | N—CH₂CH₂COOCH₃ | CF₃—C—CH₃ | NH | F | H | H | — | — | 2 | 0 |
| 89 | N—CH₂CH₂COO- | CF₃—C—CH₃ | NH | F | H | H | — | — | 2 | 0 |
| | (Cypropyl) | | | | | | | | | |
| 90 | N—CH₂CH₂COOCH₂Ph | CF₃—C—CH₃ | NH | F | H | H | — | — | 2 | 0 |
| 91 | N—CH₂CH₂COSCH₃ | CF₃—C—CH₃ | NH | F | H | H | — | — | 2 | 0 |
| 92 | N—CH₂CH₂CONHCH₃ | CF₃—C—CH₃ | NH | F | H | H | — | — | 2 | 0 |
| 93 | N—CH₂OCOCH₃ | CF₃—C—CH₃ | NH | F | H | H | — | — | 2 | 0 |
| 94 | N—CH₂OCOCypentyl | CF₃—C—CH₃ | NH | F | H | H | — | — | 2 | 0 |
| 95 | N—CH₂OCOBenzyl | CF₃—C—CH₃ | NH | F | H | H | — | — | 2 | 0 |
| 96 | N—CH₂OCH(CH₃)₂ | CF₃—C—CH₃ | NH | F | H | H | — | — | 2 | 0 |
| 97 | N—CH₂OBenzyl | CF₃—C—CH₃ | NH | F | H | H | — | — | 2 | 0 |
| 98 | N—CH₂SCH(CH₃)₂ | CF₃—C—CH₃ | NH | F | H | H | — | — | 2 | 0 |
| 99 | N—CH₂SCOCH₃ | CF₃—C—CH₃ | NH | F | H | H | — | — | 2 | 0 |
| 100 | N—CH₂SCOCypentyl | CF₃—C—CH₃ | NH | F | H | H | — | — | 2 | 0 |
| 101 | N—COCH₃ | CH₂ | NCH₃ | F | H | H | — | — | 2 | 0 |
| 102 | N—CH₂CO- | CF₃—C—CH₃ | NCH₃ | F | H | H | — | — | 2 | 0 |
| | (2,4-diCl)-Ph | | | | | | | | | |
| 103 | N—COButyl | CF₃—C—CH₃ | NCH₃ | F | H | H | — | — | 2 | 0 |
| 104 | N—CH₂COCH₃ | CF₃—C—CH₃ | NCH₃ | F | H | H | — | — | 2 | 0 |
| 105 | N—CH₂COCypentyl | CF₃—C—CH₃ | NCH₃ | F | H | H | — | — | 2 | 0 |
| 106 | N—CH₂CO-(4-F)-Ph | CF₃—C—CH₃ | NCH₃ | F | H | H | — | — | 2 | 0 |
| 107 | N—CH₂CO-2- | CF₃—C—CH₃ | NCH₃ | F | H | H | — | — | 2 | 0 |
| | Thiazolyl | | | | | | | | | |
| 108 | N—CH₂CH₂COOCH₃ | CF₃—C—CH₃ | NCH₃ | F | H | H | — | — | 2 | 0 |
| 109 | N—CH₂CH₂COO- | CF₃—C—CH₃ | NCH₃ | F | H | H | — | — | 2 | 0 |
| | (Cypropyl) | | | | | | | | | |
| 110 | N—CH₂CH₂COOCH₂Ph | CF₃—C—CH₃ | NCH₃ | F | H | H | — | — | 2 | 0 |
| 111 | N—CH₂CH₂COSCH₃ | CF₃—C—CH₃ | NCH₃ | F | H | H | — | — | 2 | 0 |
| 112 | N—CH₂CH₂CONHCH₃ | CF₃—C—CH₃ | NCH₃ | F | H | H | — | — | 2 | 0 |
| 113 | N—CH₂OCOCH₃ | CF₃—C—CH₃ | NCH₃ | F | H | H | — | — | 2 | 0 |
| 114 | N—CH₂OCOCypentyl | CF₃—C—CH₃ | NCH₃ | F | H | H | — | — | 2 | 0 |
| 115 | N—CH₂OCOBenzyl | CF₃—C—CH₃ | NCH₃ | F | H | H | — | — | 2 | 0 |
| 116 | N—CH₂OCH(CH₃)₂ | CF₃—C—CH₃ | NCH₃ | F | H | H | — | — | 2 | 0 |
| 117 | N—CH₂OBenzyl | CF₃—C—CH₃ | NCH₃ | F | H | H | — | — | 2 | 0 |
| 118 | N—CH₂SCH(CH₃)₂ | CF₃—C—CH₃ | NCH₃ | F | H | H | — | — | 2 | 0 |
| 119 | N—CH₂SCOCH₃ | CF₃—C—CH₃ | NCH₃ | F | H | H | — | — | 2 | 0 |
| 120 | N—CH₂SCOCypentyl | CF₃—C—CH₃ | NCH₃ | F | H | H | — | — | 2 | 0 |
| 121 | N—COCH₃ | CH₂ | S | F | H | H | CH₃ | CH₃ | 2 | 1 |
| 122 | N—COCH₃ | CH₂ | NCH(CH₃)₂ | F | H | H | — | — | 4 | 1 |
| 123 | N—COCH₃ | CH₂ | S | F | CH₃ | CH₃ | CH₃ | CH₃ | 2 | 1 |
| 124 | N—COCH₃ | CH₂ | S(O) | F | H | H | CH₃ | CH₃ | 2 | 1 |
| 125 | N—COCH₃ | CH₂ | S(O) | F | CH₃ | CH₃ | CH₃ | CH₃ | 2 | 1 |
| 126 | N—COCH₃ | CH₂ | S(O)₂ | F | H | H | CH₃ | CH₃ | 2 | 1 |
| 127 | N—COCH₃ | CH₂ | S(O)₂ | F | CH₃ | CH₃ | CH₃ | CH₃ | 2 | 1 |
| 128 | N—COCH₃ | CH₂ | CH₂ | F | H | H | CH₃ | CH₃ | 2 | 1 |
| 129 | N—COCH₃ | CH₂ | CH₂ | F | CH₃ | CH₃ | CH₃ | CH₃ | 2 | 1 |
| 130 | N—COCH₃ | CH₂ | CH₂ | F | H | H | H | H | 2 | 1 |
| 131 | N—COOCH₃ | CH₂ | S | F | H | H | CH₃ | CH₃ | 2 | 1 |
| 132 | N—COOCH₃ | CH₂ | S | F | CH₃ | CH₃ | CH₃ | CH₃ | 2 | 1 |
| 133 | N—COOCH₃ | CH₂ | S(O) | F | H | H | CH₃ | CH₃ | 2 | 1 |
| 134 | N—COOCH₃ | CH₂ | S(O) | F | CH₃ | CH₃ | CH₃ | CH₃ | 2 | 1 |
| 135 | N—COOCH₃ | CH₂ | S(O)₂ | F | H | H | CH₃ | CH₃ | 2 | 1 |
| 136 | N—COOCH₃ | CH₂ | S(O)₂ | F | CH₃ | CH₃ | CH₃ | CH₃ | 2 | 1 |
| 137 | N—COOCH₃ | CH₂ | CH₂ | F | H | H | CH₃ | CH₃ | 2 | 1 |
| 138 | N—COOCH₃ | CH₂ | CH₂ | F | CH₃ | CH₃ | CH₃ | CH₃ | 2 | 1 |
| 139 | N—COOCH₃ | CH₂ | CH₂ | F | H | H | H | H | 2 | 1 |
| 140 | N—COCH₃ | CHCH₃ | S | F | H | H | CH₃ | CH₃ | 2 | 1 |
| 141 | N—COCH₃ | CHCH₃ | S | F | CH₃ | CH₃ | CH₃ | CH₃ | 2 | 1 |
| 142 | N—COCH₃ | CHCH₃ | S(O) | F | H | H | CH₃ | CH₃ | 2 | 1 |
| 143 | N—COCH₃ | CHCH₃ | S(O) | F | CH₃ | CH₃ | CH₃ | CH₃ | 2 | 1 |
| 144 | N—COCH₃ | CHCH₃ | S(O)₂ | F | H | H | CH₃ | CH₃ | 2 | 1 |
| 145 | N—COCH₃ | CHCH₃ | S(O)₂ | F | CH₃ | CH₃ | CH₃ | CH₃ | 2 | 1 |
| 146 | N—COCH₃ | CHCH₃ | CH₂ | F | H | H | CH₃ | CH₃ | 2 | 1 |
| 147 | N—COCH₃ | CHCH₃ | CH₂ | F | CH₃ | CH₃ | CH₃ | CH₃ | 2 | 1 |
| 148 | N—COCH₃ | CHCH₃ | CH₂ | F | H | H | H | H | 2 | 1 |
| 149 | N—COOCH₃ | CHCH₃ | S | F | H | H | CH₃ | CH₃ | 2 | 1 |
| 150 | N—COOCH₃ | CHCH₃ | S | F | CH₃ | CH₃ | CH₃ | CH₃ | 2 | 1 |
| 151 | N—COOCH₃ | CHCH₃ | S(O) | F | H | H | CH₃ | CH₃ | 2 | 1 |
| 152 | N—COOCH₃ | CHCH₃ | S(O) | F | CH₃ | CH₃ | CH₃ | CH₃ | 2 | 1 |
| 153 | N—COOCH₃ | CHCH₃ | S(O)₂ | F | H | H | CH₃ | CH₃ | 2 | 1 |
| 154 | N—COOCH₃ | CHCH₃ | S(O)₂ | F | CH₃ | CH₃ | CH₃ | CH₃ | 2 | 1 |
| 155 | N—COOCH₃ | CHCH₃ | CH₂ | F | H | H | CH₃ | CH₃ | 2 | 1 |
| 156 | N—COOCH₃ | CHCH₃ | CH₂ | F | CH₃ | CH₃ | CH₃ | CH₃ | 2 | 1 |
| 157 | N—COOCH₃ | CHCH₃ | CH₂ | F | H | H | H | H | 2 | 1 |
| 158 | N—COCH₃ | C(CH₃)₂ | S | F | H | H | CH₃ | CH₃ | 2 | 1 |
| 159 | N—COCH₃ | C(CH₃)₂ | S | F | CH₃ | CH₃ | CH₃ | CH₃ | 2 | 1 |
| 160 | N—COCH₃ | C(CH₃)₂ | S(O) | F | H | H | CH₃ | CH₃ | 2 | 1 |
| 161 | N—COCH₃ | C(CH₃)₂ | S(O) | F | CH₃ | CH₃ | CH₃ | CH₃ | 2 | 1 |
| 162 | N—COCH₃ | C(CH₃)₂ | S(O)₂ | F | H | H | CH₃ | CH₃ | 2 | 1 |
| 163 | N—COCH₃ | C(CH₃)₂ | S(O)₂ | F | CH₃ | CH₃ | CH₃ | CH₃ | 2 | 1 |
| 164 | N—COCH₃ | C(CH₃)₂ | CH₂ | F | H | H | CH₃ | CH₃ | 2 | 1 |
| 165 | N—COCH₃ | C(CH₃)₂ | CH₂ | F | CH₃ | CH₃ | CH₃ | CH₃ | 2 | 1 |
| 166 | N—COCH₃ | C(CH₃)₂ | CH₂ | F | H | H | H | H | 2 | 1 |
| 167 | N—COOCH₃ | C(CH₃)₂ | S | F | H | H | CH₃ | CH₃ | 2 | 1 |
| 168 | N—COOCH₃ | C(CH₃)₂ | S | F | CH₃ | CH₃ | CH₃ | CH₃ | 2 | 1 |
| 169 | N—COOCH₃ | C(CH₃)₂ | S(O) | F | H | H | CH₃ | CH₃ | 2 | 1 |
| 170 | N—COOCH₃ | C(CH₃)₂ | S(O) | F | CH₃ | CH₃ | CH₃ | CH₃ | 2 | 1 |
| 171 | N—COOCH₃ | C(CH₃)₂ | S(O)₂ | F | H | H | CH₃ | CH₃ | 2 | 1 |
| 172 | N—COOCH₃ | C(CH₃)₂ | S(O)₂ | F | CH₃ | CH₃ | CH₃ | CH₃ | 2 | 1 |
| 172 | N—COOCH₃ | C(CH₃)₂ | CH₂ | F | H | H | CH₃ | CH₃ | 2 | 1 |
| 173 | N—COOCH₃ | C(CH₃)₂ | CH₂ | F | CH₃ | CH₃ | CH₃ | CH₃ | 2 | 1 |
| 174 | N—COOCH₃ | C(CH₃)₂ | CH₂ | F | H | H | H | H | 2 | 1 |
| 175 | N—COCH₃ | C═O | S | F | H | H | — | — | 4 | 0 |
| 176 | N—COCH₃ | C—(CH₃)₂ | S | F | H | H | — | — | 4 | 0 |
| 177 | N—COCH₃ | CH₂ | S(O)₂ | F | H | H | — | — | 4 | 0 |
| 178 | N—COCH₃ | CH—CH₃ | S | F | H | H | — | — | 4 | 0 |
| 179 | N—COCF₃ | CH₂ | S | F | H | H | — | — | 4 | 0 |
| 180 | N— | CH₂ | S | F | H | H | — | — | 4 | 0 |
| | CO(Cypropyl) | | | | | | | | | |
| 181 | N—COCH₃ | C—(CH₃)₂ | S | F | CH₃ | CH₃ | — | — | 4 | 0 |
| 182 | N—COCH₃ | CH₃—C—COOCH₃ | S | F | H | H | — | — | 4 | 0 |
| 183 | N—COCH₃ | CH- | S | F | H | H | — | — | 4 | 0 |
| | | (1R,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 184 | N—COCH₃ | CH-(4F)- | S | F | H | H | — | — | 4 | 0 |
| | | phenyl | | | | | | | | |
| 185 | N—COC(CH₃)₃ | CH₂ | S | F | H | H | — | — | 4 | 0 |
| 186 | N—COCH₂CH(CH₃)₂ | CH₂ | S | F | H | H | — | — | 4 | 0 |
| 187 | N—CO(Cypentyl) | CH₂ | S | F | H | H | — | — | 4 | 0 |
| 188 | N—COCH₃ | CH—CF₃ | S | F | H | H | — | — | 4 | 0 |
| 189 | N—COOC(CH₃)₃ | C—(CH₃)₂ | O | F | H | H | — | — | 4 | 0 |
| 190 | N—COCH₃ | CF₃—C—CH₃ | O | F | H | H | — | — | 4 | 0 |
| 191 | N—COCH₃ | CH₂ | O | F | H | H | — | — | 4 | 0 |
| 192 | N—COCH₃ | CH—CF₃ | O | F | H | H | — | — | 4 | 0 |
| 193 | N—COCH₃ | CH-Cypentyl | S | F | H | H | — | — | 4 | 0 |
| 194 | N—COCH₃ | CH-Cypropyl | S | F | H | H | — | — | 4 | 0 |
| 195 | N—COSCH(CH₃)₂ | CH₂ | S | F | H | H | — | — | 4 | 0 |
| 196 | N—COCH₃ | CH₂ | S | F | H | H | H | H | 4 | 1 |
| 197 | N—COCH₃ | CH₂ | S | F | H | H | — | — | 4 | 0 |
| 198 | N—COCH₃ | CH₂ | S(O) | F | H | H | — | — | 4 | 0 |
| 199 | N—CH₂COCH₃ | CH₂ | S | F | H | H | — | — | 4 | 0 |
| 200 | N—CH₂CO(Cypentyl | CH₂ | S | F | H | H | — | — | 4 | 0 |
| 201 | N—CH₂CO-(4-F)-Ph | CH₂ | S | F | H | H | — | — | 4 | 0 |
| 202 | N—CH₂CO-2- | CH₂ | S | F | H | H | — | — | 4 | 0 |
| | thiazolyl | | | | | | | | | |
| 203 | N—CH₂CH₂COOCH₃ | CH₂ | S | F | H | H | — | — | 4 | 0 |
| 204 | N—CH₂CH₂COO- | CH₂ | S | F | H | H | — | — | 4 | 0 |
| | (Cypropyl) | | | | | | | | | |
| 205 | N—CH₂CH₂COOCH₂Ph | CH₂ | S | F | H | H | — | — | 4 | 0 |
| 206 | N—CH₂CH₂COSCH₃ | CH₂ | S | F | H | H | — | — | 4 | 0 |
| 207 | N—CH₂CH₂CONHCH₃ | CH₂ | S | F | H | H | — | — | 4 | 0 |
| 208 | N—CH₂OCOCH₃ | CH₂ | S | F | H | H | — | — | 4 | 0 |
| 209 | N—CH₂OCOCypentyl | CH₂ | S | F | H | H | — | — | 4 | 0 |
| 210 | N—CH₂OCObenzyl | CH₂ | S | F | H | H | — | — | 4 | 0 |
| 211 | N—CH₂OCH(CH₃)₂ | CH₂ | S | F | H | H | — | — | 4 | 0 |
| 212 | N—CH₂Obenzyl | CH₂ | S | F | H | H | — | — | 4 | 0 |
| 213 | N—CH₂SCH(CH₃)₂ | CH₂ | S | F | H | H | — | — | 4 | 0 |
| 214 | N—CH₂SCOCH₃ | CH₂ | S | F | H | H | — | — | 4 | 0 |
| 215 | N—CH₂SCOCypentyl | CH₂ | S | F | H | H | — | — | 4 | 0 |
| 216 | N—COCH₃ | CH(CH₃)₂ | S(O) | F | H | H | — | — | 4 | 0 |
| 217 | N—COCH₃ | CH(CH₃)₂ | S(O)2 | F | H | H | — | — | 4 | 0 |
| 218 | N—CH₂COCH₃ | CH(CH₃)₂ | S | F | H | H | — | — | 4 | 0 |
| 219 | N—CH₂COCypentyl | CH(CH₃)₂ | S | F | H | H | — | — | 4 | 0 |
| 220 | N—CH₂CO-(4-F)-Ph | CH(CH₃)₂ | S | F | H | H | — | — | 4 | 0 |
| 221 | N—CH₂CO-2-Thiazolyl | CH(CH₃)₂ | S | F | H | H | — | — | 4 | 0 |
| 222 | N—CH₂CH₂COOCH₃ | CH(CH₃)₂ | S | F | H | H | — | — | 4 | 0 |
| 223 | N—CH₂CH₂COO- | CH(CH₃)₂ | S | F | H | H | — | — | 4 | 0 |
| | (Cypropyl) | | | | | | | | | |
| 224 | N—CH₂CH₂COOCH₂Ph | CH(CH₃)₂ | S | F | H | H | — | — | 4 | 0 |
| 225 | N—CH₂CH₂COSCH₃ | CH(CH₃)₂ | S | F | H | H | — | — | 4 | 0 |
| 226 | N—CH₂CH₂CONHCH₃ | CH(CH₃)₂ | S | F | H | H | — | — | 4 | 0 |
| 227 | N—CH₂OCOCH₃ | CH(CH₃)₂ | S | F | H | H | — | — | 4 | 0 |
| 228 | N—CH₂OCO(Cypentyl) | CH(CH₃)₂ | S | F | H | H | — | — | 4 | 0 |
| 229 | N—CH₂OCO(Benzyl) | CH(CH₃)₂ | S | F | H | H | — | — | 4 | 0 |
| 230 | N—CH₂OCH(CH₃)₂ | CH(CH₃)₂ | S | F | H | H | — | — | 4 | 0 |
| 231 | N—CH₂O(Benzyl) | CH(CH₃)₂ | S | F | H | H | — | — | 4 | 0 |
| 232 | N—CH₂SCH(CH₃)₂ | CH(CH₃)₂ | S | F | H | H | — | — | 4 | 0 |
| 233 | N—CH₂SCOCH₃ | CH(CH₃)₂ | S | F | H | H | — | — | 4 | 0 |
| 234 | N—CH₂SCO(Cypentyl) | CH(CH₃)₂ | S | F | H | H | — | — | 4 | 0 |
| 235 | N—CH₂CO- | CF₃—C—CH₃ | O | F | H | H | — | — | 4 | 0 |
| | (2,4-diCl)-Ph | | | | | | | | | |
| 236 | N-COButyl | CF₃—C—CH₃ | O | F | H | H | — | — | 4 | 0 |
| 237 | N—CH₂COCH₃ | CF₃—C—CH₃ | O | F | H | H | — | — | 4 | 0 |
| 238 | N—CH₂CO(Cypentyl) | CF₃—C—CH₃ | O | F | H | H | — | — | 4 | 0 |
| 239 | N—CH₂CO-(4-F)-Ph | CF₃—C—CH₃ | O | F | H | H | — | — | 4 | 0 |
| 240 | N—CH₂CO-2- | CF₃—C—CH₃ | O | F | H | H | — | — | 4 | 0 |
| | Thiazolyl | | | | | | | | | |
| 241 | N—CH₂CH₂COOCH₃ | CF₃—C—CH₃ | O | F | H | H | — | — | 4 | 0 |
| 242 | N—CH₂CH₂COO- | CF₃—C—CH₃ | O | F | H | H | — | — | 4 | 0 |
| | (Cypropyl) | | | | | | | | | |
| 243 | N—CH₂CH₂COOCH₂Ph | CF₃—C—CH₃ | O | F | H | H | — | — | 4 | 0 |
| 244 | N—CH₂CH₂COSCH₃ | CF₃—C—CH₃ | O | F | H | H | — | — | 4 | 0 |
| 245 | N—CH₂CH₂CONHCH₃ | CF₃—C—CH₃ | O | F | H | H | — | — | 4 | 0 |
| 246 | N—CH₂OCOCH₃ | CF₃—C—CH₃ | O | F | H | H | — | — | 4 | 0 |
| 247 | N—CH₂OCO(Cypentyl) | CF₃—C—CH₃ | O | F | H | H | — | — | 4 | 0 |
| 248 | N—CH₂OCOBenzyl | CF₃—C—CH₃ | O | F | H | H | — | — | 4 | 0 |
| 249 | N—CH₂OCH(CH₃)₂ | CF₃—C—CH₃ | O | F | H | H | — | — | 4 | 0 |
| 250 | N—CH₂OBenzyl | CF₃—C—CH₃ | O | F | H | H | — | — | 4 | 0 |
| 251 | N—CH₂SCH(CH₃)₂ | CF₃—C—CH₃ | O | F | H | H | — | — | 4 | 0 |
| 252 | N—CH₂SCOCH₃ | CF₃—C—CH₃ | O | F | H | H | — | — | 4 | 0 |
| 253 | N—CH₂SCO(Cypentyl) | CF₃—C—CH₃ | S | F | H | H | — | — | 4 | 0 |
| 254 | N—COCH₃ | CH₂ | NH | F | H | H | — | — | 4 | 0 |
| 255 | N—CH₂CO- | CF₃—C—CH₃ | NH | F | H | H | — | — | 4 | 0 |
| | (2,4-diCl)-Ph | | | | | | | | | |
| 256 | N-COButyl | CF₃—C—CH₃ | NH | F | H | H | — | — | 4 | 0 |
| 257 | N—CH₂COCH₃ | CF₃—C—CH₃ | NH | F | H | H | — | — | 4 | 0 |
| 258 | N—CH₂COCypentyl | CF₃—C—CH₃ | NH | F | H | H | — | — | 4 | 0 |
| 259 | N—CH₂CO-(4-F)-Ph | CF₃—C—CH₃ | NH | F | H | H | — | — | 4 | 0 |
| 260 | N—CH₂CO-2- | CF₃—C—CH₃ | NH | F | H | H | — | — | 4 | 0 |
| | Thiazolyl | | | | | | | | | |
| 261 | N—CH₂CH₂COOCH₃ | CF₃—C—CH₃ | NH | F | H | H | — | — | 4 | 0 |
| 262 | N—CH₂CH₂COO- | CF₃—C—CH₃ | NH | F | H | H | — | — | 4 | 0 |
| | (Cypropyl) | | | | | | | | | |
| 263 | N—CH₂CH₂COOCH₂Ph | CF₃—C—CH₃ | NH | F | H | H | — | — | 4 | 0 |
| 264 | N—CH₂CH₂COSCH₃ | CF₃—C—CH₃ | NH | F | H | H | — | — | 4 | 0 |
| 265 | N—CH₂CH₂CONHCH₃ | CF₃—C—CH₃ | NH | F | H | H | — | — | 4 | 0 |
| 266 | N—CH₂OCOCH₃ | CF₃—C—CH₃ | NH | F | H | H | — | — | 4 | 0 |
| 267 | N—CH₂OCO(Cypentyl) | CF₃—C—CH₃ | NH | F | H | H | — | — | 4 | 0 |
| 268 | N—CH₂OCO(Benzyl) | CF₃—C—CH₃ | NH | F | H | H | — | — | 4 | 0 |
| 269 | N— | CF₃—C—CH₃ | NH | F | H | H | — | — | 4 | 0 |
| | CH₂OCH(CH₃)₂ | | | | | | | | | |
| 270 | N—CH₂OBenzyl | CF₃—C—CH₃ | NH | F | H | H | — | — | 4 | 0 |
| 271 | N— | CF₃—C—CH₃ | NH | F | H | H | — | — | 4 | 0 |
| | CH₂SCH(CH₃)₂ | | | | | | | | | |
| 272 | N—CH₂SCOCH₃ | CF₃—C—CH₃ | NH | F | H | H | — | — | 4 | 0 |
| 273 | N— | CF₃—C—CH₃ | NH | F | H | H | — | — | 4 | 0 |
| | CH₂SCO (Cypentyl | | | | | | | | | |
| tyl) | | | | | | | | | | |
| 274 | N—COCH₃ | CH₂ | NCH₃ | F | H | H | — | — | 4 | 0 |
| 275 | N—CH₂CO- | CF₃—C—CH₃ | NCH₃ | F | H | H | — | — | 4 | 0 |
| | (2,4-diCl)-Ph | | | | | | | | | |
| 276 | N-COButyl | CF₃—C—CH₃ | NCH₃ | F | H | H | — | — | 4 | 0 |
| 277 | N—CH₂COCH₃ | CF₃—C—CH₃ | NCH₃ | F | H | H | — | — | 4 | 0 |
| 278 | N— | CF₃—C—CH₃ | NCH₃ | F | H | H | — | — | 4 | 0 |
| | CH₂CO(Cypentyl) | | | | | | | | | |
| 279 | N—CH₂CO-(4-F)-Ph | CF₃—C—CH₃ | NCH₃ | F | H | H | — | — | 4 | 0 |
| 280 | N—CH₂CO-2- | CF₃—C—CH₃ | NCH₃ | F | H | H | — | — | 4 | 0 |
| | Thiazolyl | | | | | | | | | |
| 281 | N— | CF₃—C—CH₃ | NCH₃ | F | H | H | — | — | 4 | 0 |
| | CH₂CH₂COOCH₃ | | | | | | | | | |
| 282 | N—CH₂CH₂COO- | CF₃—C—CH₃ | NCH₃ | F | H | H | — | — | 4 | 0 |
| | (Cypropyl) | | | | | | | | | |
| 283 | N— | CF₃—C—CH₃ | NCH₃ | F | H | H | — | — | 4 | 0 |
| | CH₂CH₂COOCH₂Ph | | | | | | | | | |
| 284 | N— | CF₃—C—CH₃ | NCH₃ | F | H | H | — | — | 4 | 0 |
| | CH₂CH₂COSCH₃ | | | | | | | | | |
| 285 | N— | CF₃—C—CH₃ | NCH₃ | F | H | H | — | — | 4 | 0 |
| | CH₂CH₂CONHCH₃ | | | | | | | | | |
| 286 | N—CH₂OCOCH₃ | CF₃—C—CH₃ | NCH₃ | F | H | H | — | — | 4 | 0 |
| 287 | N— | CF₃—C—CH₃ | NCH₃ | F | H | H | — | — | 4 | 0 |
| | CH₂OCO(Cypentyl) | | | | | | | | | |
| 288 | N— | CF₃—C—CH₃ | NCH₃ | F | H | H | — | — | 4 | 0 |
| | CH₂OCOBenzyl | | | | | | | | | |
| 289 | N— | CF₃—C—CH₃ | NCH₃ | F | H | H | — | — | 4 | 0 |
| | CH₂OCH(CH₃)₂ | | | | | | | | | |
| 290 | N—CH₂Benzyl | CF₃—C—CH₃ | NCH₃ | F | H | H | — | — | 4 | 0 |
| 291 | N—COCH₃ | CH₂ | NCH(CH₃)₂ | F | H | H | — | — | 4 | 0 |
| 292 | N— | CF₃—C—CH₃ | NCH₃ | F | H | H | — | — | 4 | 0 |
| | CH₂SCH(CH₃)₂ | | | | | | | | | |
| 293 | N—CH₂SCOCH₃ | CF₃—C—CH₃ | NCH₃ | F | H | H | — | — | 4 | 0 |
| 294 | N— | CF₃—C—CH₃ | NCH₃ | F | H | H | — | — | 4 | 0 |
| | CH₂SCOCypentyl | | | | | | | | | |
| 295 | N—COCH₃ | CH₂ | S | F | H | H | CH₃ | CH₃ | 4 | 1 |
| 296 | N—COCH₃ | CH₂ | S | F | CH₃ | CH₃ | CH₃ | CH₃ | 4 | 1 |
| 297 | N—COCH₃ | CH₂ | S(O) | F | H | H | CH₃ | CH₃ | 4 | 1 |
| 298 | N—COCH₃ | CH₂ | S(O) | F | CH₃ | CH₃ | CH₃ | CH₃ | 4 | 1 |
| 299 | N—OCH₃ | CH₂ | S(O)₂ | F | H | H | CH₃ | CH₃ | 4 | 1 |
| 300 | N—OCH₃ | CH₂ | S(O)₂ | F | CH₃ | CH₃ | CH₃ | CH₃ | 4 | 1 |
| 301 | N—OCH₃ | CH₂ | CH₂ | F | H | H | CH₃ | CH₃ | 4 | 1 |
| 302 | N—COCH₃ | CH₂ | CH₂ | F | CH₃ | CH₃ | CH₃ | CH₃ | 4 | 1 |
| 303 | N—COCH₃ | CH₂ | CH₂ | F | H | H | H | H | 4 | 1 |
| 304 | N—COOCH₃ | CH₂ | S | F | H | H | CH₃ | CH₃ | 4 | 1 |
| 305 | N—COOCH₃ | CH₂ | S | F | CH₃ | CH₃ | CH₃ | CH₃ | 4 | 1 |
| 306 | N—COOCH₃ | CH₂ | S(O) | F | H | H | CH₃ | CH₃ | 4 | 1 |
| 307 | N—COOCH₃ | CH₂ | S(O) | F | CH₃ | CH₃ | CH₃ | CH₃ | 4 | 1 |
| 308 | N—COOCH₃ | CH₂ | S(O)₂ | F | H | H | CH₃ | CH₃ | 4 | 1 |
| 309 | N—COOCH₃ | CH₂ | S(O)₂ | F | CH₃ | CH₃ | CH₃ | CH₃ | 4 | 1 |
| 310 | N—COOCH₃ | CH₂ | CH₂ | F | H | H | CH₃ | CH₃ | 4 | 1 |
| 311 | N—COOCH₃ | CH₂ | CH₂ | F | CH₃ | CH₃ | CH₃ | CH₃ | 4 | 1 |
| 312 | N—COOCH₃ | CH₂ | CH₂ | F | H | H | H | H | 4 | 1 |
| 313 | N—COCH₃ | CHCH₃ | S | F | H | H | CH₃ | CH₃ | 4 | 1 |
| 314 | N—COCH₃ | CHCH₃ | S | F | CH₃ | CH₃ | CH₃ | CH₃ | 4 | 1 |
| 315 | N—COCH₃ | CHCH₃ | S(O) | F | H | H | CH₃ | CH₃ | 4 | 1 |
| 316 | N—COCH₃ | CHCH₃ | S(O) | F | CH₃ | CH₃ | CH₃ | CH₃ | 4 | 1 |
| 317 | N—COCH₃ | CHCH₃ | S(O)₂ | F | H | H | CH₃ | CH₃ | 4 | 1 |
| 318 | N—COCH₃ | CHCH₃ | S(O)₂ | F | CH₃ | CH₃ | CH₃ | CH₃ | 4 | 1 |
| 319 | N—COCH₃ | CHCH₃ | CH₂ | F | H | H | CH₃ | CH₃ | 4 | 1 |
| 320 | N—COCH₃ | CHCH₃ | CH₂ | F | CH₃ | CH₃ | CH₃ | CH₃ | 4 | 1 |
| 321 | N—COCH₃ | CHCH₃ | CH₂ | F | H | H | H | H | 4 | 1 |
| 322 | N—COOCH₃ | CHCH₃ | S | F | H | H | CH₃ | CH₃ | 4 | 1 |
| 323 | N—COOCH₃ | CHCH₃ | S | F | CH | CH₃ | CH₃ | CH₃ | 4 | 1 |
| 324 | N—COOCH₃ | CHCH₃ | S(O) | F | H | H | CH₃ | CH₃ | 4 | 1 |
| 325 | N—COOCH₃ | CHCH₃ | S(O) | F | CH | CH | CH | CH₃ | 4 | 1 |
| 326 | N—COOCH₃ | CHCH₃ | S(O)₂ | F | H | H | CH₃ | CH₃ | 4 | 1 |
| 327 | N—COOCH₃ | CHCH₃ | S(O)₂ | F | CH₃ | CH₃ | CH₃ | CH₃ | 4 | 1 |
| 328 | N—COOCH₃ | CHCH₃ | CH₂ | F | H | H | CH₃ | CH₃ | 4 | 1 |
| 329 | N—COOCH₃ | CHCH₃ | CH₂ | F | CH₃ | CH₃ | CH₃ | CH₃ | 4 | 1 |
| 330 | N—COOCH₃ | CHCH₃ | CH₂ | F | H | H | H | H | 4 | 1 |
| 331 | N—COCH₃ | C(CH₃)₂ | S | F | H | H | CH₃ | CH₃ | 4 | 1 |
| 332 | N—COCH₃ | C(CH₃)₂ | S | F | CH₃ | CH₃ | CH₃ | CH₃ | 4 | 1 |
| 333 | N—COCH₃ | C(CH₃)₂ | S(O) | F | H | H | CH₃ | CH₃ | 4 | 1 |
| 334 | N—COCH₃ | C(CH₃)₂ | S(O) | F | CH₃ | CH₃ | CH₃ | CH₃ | 4 | 1 |
| 335 | N—COCH₃ | C(CH₃)₂ | S(O)₂ | F | H | H | CH₃ | CH₃ | 4 | 1 |
| 336 | N—COCH₃ | C(CH₃)₂ | S(O)₂ | F | CH₃ | CH₃ | CH₃ | CH₃ | 4 | 1 |
| 337 | N—COCH₃ | C(CH₃)₂ | CH₂ | F | H | H | CH₃ | CH₃ | 4 | 1 |
| 338 | N—COCH₃ | C(CH₃)₂ | CH₂ | F | CH₃ | CH₃ | CH₃ | CH₃ | 4 | 1 |
| 339 | N—COCH₃ | C(CH₃)₂ | CH₂ | F | H | H | H | H | 4 | 1 |
| 340 | N—COOCH₃ | C(CH₃)₂ | S | F | H | H | CH₃ | CH₃ | 4 | 1 |
| 341 | N—COOCH₃ | C(CH₃)₂ | S | F | CH₃ | CH₃ | CH₃ | CH₃ | 4 | 1 |
| 342 | N—COOCH₃ | C(CH₃)₂ | S(O) | F | H | H | CH₃ | CH₃ | 4 | 1 |
| 343 | N—COOCH₃ | C(CH₃)₂ | S(O) | F | CH₃ | CH₃ | CH₃ | CH₃ | 4 | 1 |
| 344 | N—COOCH₃ | C(CH₃)₂ | S(O)₂ | F | H | H | CH₃ | CH₃ | 4 | 1 |
| 345 | N—COOCH₃ | C(CH₃)₂ | S(O)₂ | F | CH₃ | CH₃ | CH₃ | CH₃ | 4 | 1 |
| 346 | N—COOCH₃ | C(CH₃)₂ | CH₂ | F | H | H | CH₃ | CH₃ | 4 | 1 |
| 347 | N—COOCH₃ | C(CH₃)₂ | CH₂ | F | CH₃ | CH₃ | CH₃ | CH₃ | 4 | 1 |
| 348 | N—COOCH₃ | C(CH₃)₂ | CH₂ | F | H | H | H | H | 4 | 1 |
| 349 | N—COCH₃ | C═O | S | H | H | H | — | — | 2 | 0 |
| 350 | N—COCH₃ | C—(CH₃)₂ | S | H | H | H | — | — | 2 | 0 |
| 351 | N—COCH₃ | CH₂ | S(O)₂ | H | H | H | — | — | 2 | 0 |
| 352 | N—COCH₃ | CH—CH₃ | S | H | H | H | — | — | 2 | 0 |
| 353 | N—COCF₃ | CH₂ | S | H | H | H | — | — | 2 | 0 |
| 354 | N—CO(Cypropyl) | CH₂ | S | H | H | H | — | — | 2 | 0 |
| 355 | N—COCH₃ | C—(CH₃)₂ | S | H | CH₃ | CH₃ | — | — | 2 | 0 |
| 356 | N—COCH₃ | CH₃—C—COOCH₃ | S | H | H | H | — | — | 2 | 0 |
| 357 | N—COCH₃ | CH- | S | H | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 358 | N—COCH₃ | CH-(4F)-phenyl | S | H | H | H | — | — | 2 | 0 |
| 359 | N—COC(CH₃)₃ | CH₂ | S | H | H | H | — | — | 2 | 0 |
| 360 | N— | CH₂ | S | H | H | H | — | — | 2 | 0 |
| | COCH₂CH(CH₃)₂ | | | | | | | | | |
| 361 | N— | CH₂ | S | H | H | H | — | — | 2 | 0 |
| | CO(Cypentyl) | | | | | | | | | |
| 362 | N—COCH₃ | CH—CF₃ | S | H | H | H | — | — | 2 | 0 |
| 363 | N—COOC(CH₃)₃ | C—(CH₃)₂ | O | H | H | H | — | — | 2 | 0 |
| 364 | N—COCH₃ | CF₃—C—CH₃ | O | H | H | H | — | — | 2 | 0 |
| 365 | N—COCH₃ | CH₂ | O | H | H | H | — | — | 2 | 0 |
| 366 | N—COCH₃ | CH—CF₃ | O | H | H | H | — | — | 2 | 0 |
| 367 | N—COCH₃ | CH-Cypentyl | S | H | H | H | — | — | 2 | 0 |
| 368 | N—COCH₃ | CH-Cypropyl | S | H | H | H | — | — | 2 | 0 |
| 369 | N—COSCH(CH₃)₂ | CH₂ | S | H | H | H | — | — | 2 | 0 |
| 370 | N—COCH₃ | CH₂ | S | H | H | H | H | H | 2 | 1 |
| 371 | N—COCH₃ | CH₂ | S | H | H | H | — | — | 2 | 0 |
| 372 | N—COCH₃ | CH₂ | S(O) | H | H | H | — | — | 2 | 0 |
| 373 | N—CH₂COCH₃ | CH₂ | S | H | H | H | — | — | 2 | 0 |
| 374 | N—CH₂CO(Cypentyl | CH₂ | S | H | H | H | — | — | 2 | 0 |
| 375 | N—CH₂CO-(4-F)-Ph | CH₂ | S | H | H | H | — | — | 2 | 0 |
| 376 | N—CH₂CO-2-(Thiazolyl) | CH₂ | S | H | H | H | — | — | 2 | 0 |
| 377 | N—CH₂CH₂COOCH₃ | CH₂ | S | H | H | H | — | — | 2 | 0 |
| 378 | N—CH₂CH₂COO- | CH₂ | S | H | H | H | — | — | 2 | 0 |
| | (Cypropyl) | | | | | | | | | |
| 379 | N—CH₂CH₂COOCH₂Ph | CH₂ | S | H | H | H | — | — | 2 | 0 |
| 380 | N—CH₂CH₂COSCH₃ | CH₂ | S | H | H | H | — | — | 2 | 0 |
| 381 | N—CH₂CH₂CONHCH₃ | CH₂ | S | H | H | H | — | — | 2 | 0 |
| 382 | N—CH₂OCOCH₃ | CH₂ | S | H | H | H | — | — | 2 | 0 |
| 383 | N—CH₂OCO(Cypentyl) | CH₂ | S | H | H | H | — | — | 2 | 0 |
| 384 | N—CH₂OCO(Benzyl) | CH₂ | S | H | H | H | — | — | 2 | 0 |
| 385 | N—CH₂OCH(CH₃)₂ | CH₂ | S | H | H | H | — | — | 2 | 0 |
| 386 | N—CH₂O(Benzyl) | CH₂ | S | H | H | H | — | — | 2 | 0 |
| 387 | N—CH₂SCH(CH₃)₂ | CH₂ | S | H | H | H | — | — | 2 | 0 |
| 388 | N—CH₂SCOCH₃ | CH₂ | S | H | H | H | — | — | 2 | 0 |
| 389 | N—CH₂SCOCypentyl | CH₂ | S | H | H | H | — | — | 2 | 0 |
| 390 | N—COCH₃ | CH(CH₃)₂ | S(O) | H | H | H | — | — | 2 | 0 |
| 391 | N—COCH₃ | CH(CH₃)₂ | S(O)₂ | H | H | H | — | — | 2 | 0 |
| 392 | N—CH₂COCH₃ | CH(CH₃)₂ | S | H | H | H | — | — | 2 | 0 |
| 393 | N—CH₂COCypentyl | CH(CH₃)₂ | S | H | H | H | — | — | 2 | 0 |
| 394 | N—CH₂CO-(4-F)-Ph | CH(CH₃)₂ | S | H | H | H | — | — | 2 | 0 |
| 395 | N—CH₂CO-2-thiazolyl | CH(CH₃)₂ | S | H | H | H | — | — | 2 | 0 |
| 396 | N—CH₂CH₂COOCH₃ | CH(CH₃)₂ | S | H | H | H | — | — | 2 | 0 |
| 397 | N—CH₂CH₂COO- | CH(CH₃)₂ | S | H | H | H | — | — | 2 | 0 |
| | (Cypropyl) | | | | | | | | | |
| 398 | N—CH₂CH₂COOCH₂Ph | CH(CH₃)₂ | S | H | H | H | — | — | 2 | 0 |
| 399 | N—CH₂CH₂COSCH₃ | CH(CH₃)₂ | S | H | H | H | — | — | 2 | 0 |
| 400 | N—CH₂CH₂CONHCH₃ | CH(CH₃)₂ | S | H | H | H | — | — | 2 | 0 |
| 401 | N—CH₂OCOCH₃ | CH(CH₃)₂ | S | H | H | H | — | — | 2 | 0 |
| 402 | N—CH₂OCO(Cypentyl) | CH(CH₃)₂ | S | H | H | H | — | — | 2 | 0 |
| 403 | N—CH₂OCO(Benzyl) | CH(CH₃)₂ | S | H | H | H | — | — | 2 | 0 |
| 404 | N—CH₂OCH(CH₃)₂ | CH(CH₃)₂ | S | H | H | H | — | — | 2 | 0 |
| 405 | N—CH₂O(Benzyl) | CH(CH₃)₂ | S | H | H | H | — | — | 2 | 0 |
| 406 | N—CH₂SCH(CH₃)₂ | CH(CH₃)₂ | S | H | H | H | — | — | 2 | 0 |
| 407 | N—CH₂SCOCH₃ | CH(CH₃)₂ | S | H | H | H | — | — | 2 | 0 |
| 408 | N—CH₂SCO(Cypentyl) | CH(CH₃)₂ | S | H | H | H | — | — | 2 | 0 |
| 409 | N—CH₂CO- | CF₃—C—CH₃ | O | H | H | H | — | — | 2 | 0 |
| | (2,4-diCl)-Ph | | | | | | | | | |
| 410 | N—CO(Butyl) | CF₃—C—CH₃ | O | H | H | H | — | — | 2 | 0 |
| 411 | N—CH₂COCH₃ | CF₃—C—CH₃ | O | H | H | H | — | — | 2 | 0 |
| 412 | N—CH₂CO(Cypentyl | CF₃—C—CH₃ | O | H | H | H | — | — | 2 | 0 |
| 413 | N—CH₂CO-(4-F)-Ph | CF₃—C—CH₃ | O | H | H | H | — | — | 2 | 0 |
| 414 | N—CH2CO-2- | CF₃—C—CH₃ | O | H | H | H | — | — | 2 | 0 |
| | Thiazolyl | | | | | | | | | |
| 415 | N— | CF₃—C—CH₃ | O | H | H | H | — | — | 2 | 0 |
| | CH₂CH₂COOCH₃ | | | | | | | | | |
| 416 | N—CH₂CH₂COO- | CF₃—C—CH₃ | O | H | H | H | — | — | 2 | 0 |
| | (Cypropyl) | | | | | | | | | |
| 417 | N—CH₂CH₂COOCH₂Ph | CF₃—C—CH₃ | O | H | H | H | — | — | 2 | 0 |
| 418 | N—CH₂CH₂COSCH₃ | CF₃—C—CH₃ | O | H | H | H | — | — | 2 | 0 |
| 419 | N—CH₂CH₂CONHCH₃ | CF₃—C—CH₃ | O | H | H | H | — | — | 2 | 0 |
| 420 | N—CH₂OCOCH₃ | CF₃—C—CH₃ | O | H | H | H | — | — | 2 | 0 |
| 421 | N— | CF₃—C—CH₃ | O | H | H | H | — | — | 2 | 0 |
| | CH₂OCO(Cypentyl) | | | | | | | | | |
| 422 | N— | CF₃—C—CH₃ | O | H | H | H | — | — | 2 | 0 |
| | CH₂OCO(Benzyl) | | | | | | | | | |
| 423 | N— | CF₃—C—CH₃ | O | H | H | H | — | — | 2 | 0 |
| | CH₂OCH(CH₃)₂ | | | | | | | | | |
| 424 | N— | CF₃—C—CH₃ | O | H | H | H | — | — | 2 | 0 |
| | CH₂OBenzile | | | | | | | | | |
| 425 | N— | CF₃—C—CH₃ | O | H | H | H | — | — | 2 | 0 |
| | CH₂SCH(CH₃)₂ | | | | | | | | | |
| 426 | N—CH₂SCOCH₃ | CF₃—C—CH₃ | O | H | H | H | — | — | 2 | 0 |
| 427 | N— | CF₃—C—CH₃ | S | H | H | H | — | — | 2 | 0 |
| | CH₂SCO(Cypentyl) | | | | | | | | | |
| 428 | N—COCH₃ | CH₂ | NH | G | H | H | — | — | 2 | 0 |
| | | | | H | | | | | | |
| 429 | N—CH₂CO- | CF₃—C—CH₃ | NH | H | H | H | — | — | 2 | 0 |
| | (2,4-diCl)-Ph | | | | | | | | | |
| 430 | N—COButyl | CF₃—C—CH₃ | NH | H | H | H | — | — | 2 | 0 |
| 431 | N—CH₂COCH₃ | CF₃—C—CH₃ | NH | H | H | H | — | — | 2 | 0 |
| 432 | N— | CF₃—C—CH₃ | NH | H | H | H | — | — | 2 | 0 |
| | CH₂CO(Cypentyl) | | | | | | | | | |
| 433 | N—CH₂CO-(4-F)-Ph | CF₃—C—CH₃ | NH | H | H | H | — | — | 2 | 0 |
| 434 | N—CH₂CO-2- | CF₃—C—CH₃ | NH | H | H | H | — | — | 2 | 0 |
| | thiazolyl | | | | | | | | | |
| 435 | N— | CF₃—C—CH₃ | NH | H | H | H | — | — | 2 | 0 |
| | CH₂CH₂COOCH₃ | | | | | | | | | |
| 436 | N—CH₂CH₂COO- | CF₃—C—CH₃ | NH | H | H | H | — | — | 2 | 0 |
| | (Cypropyl) | | | | | | | | | |
| 437 | N— | CF₃—C—CH₃ | NH | H | H | H | — | — | 2 | 0 |
| | CH₂CH₂COOCH₂Ph | | | | | | | | | |
| 438 | N— | CF₃—C—CH₃ | NH | H | H | H | — | — | 2 | 0 |
| | CH₂CH₂COSCH₃ | | | | | | | | | |
| 439 | N— | CF₃—C—CH₃ | NH | H | H | H | — | — | 2 | 0 |
| | CH₂CH₂CONHCH₃ | | | | | | | | | |
| 440 | N—CH₂OCOCH₃ | CF₃—C—CH₃ | NH | H | H | H | — | — | 2 | 0 |
| 441 | N— | CF₃—C—CH₃ | NH | H | H | H | — | — | 2 | 0 |
| | CH₂OCO(Cypentyl) | | | | | | | | | |
| 442 | N— | CF₃—C—CH₃ | NH | H | H | H | — | — | 2 | 0 |
| | CH₂OCO(Benzyl) | | | | | | | | | |
| 443 | N— | CF₃—C—CH₃ | NH | H | H | H | — | — | 2 | 0 |
| | CH₂OCH(CH₃)₂ | | | | | | | | | |
| 444 | N— | CF₃—C—CH₃ | NH | H | H | H | — | — | 2 | 0 |
| | CH₂O(Benzyl) | | | | | | | | | |
| 445 | N— | CF₃—C—CH₃ | NH | H | H | H | — | — | 2 | 0 |
| | CH₂SCH(CH₃)₂ | | | | | | | | | |
| 446 | N—CH₂SCOCH₃ | CF₃—C—CH₃ | NH | H | H | H | — | — | 2 | 0 |
| 447 | N— | CF₃—C—CH₃ | NH | H | H | H | — | — | 2 | 0 |
| | CH₂SCOCypentyl | | | | | | | | | |
| 448 | N—COCH₃ | CH₂ | NCH₃ | H | H | H | — | — | 2 | 0 |
| 449 | N—CH₂CO- | CF₃—C—CH₃ | NCH₃ | H | H | H | — | — | 2 | 0 |
| | (2,4-diCl)-Ph | | | | | | | | | |
| 450 | N-COButyl | CF₃—C—CH₃ | NCH₃ | H | H | H | — | — | 2 | 0 |
| 451 | N—CH₂COCH₃ | CF₃—C—CH₃ | NCH₃ | H | H | H | — | — | 2 | 0 |
| 452 | N— | CF₃—C—CH₃ | NCH₃ | H | H | H | — | — | 2 | 0 |
| | CH₂COCypentyl | | | | | | | | | |
| 453 | N—CH₂CO-(4- | CF₃—C—CH₃ | NCH₃ | H | H | H | — | — | 2 | 0 |
| | F)-Ph | | | | | | | | | |
| 454 | N—CH₂CO-2- | CF₃—C—CH₃ | NCH₃ | H | H | H | — | — | 2 | 0 |
| | thiazolyl | | | | | | | | | |
| 455 | N— | CF₃—C—CH₃ | NCH₃ | H | H | H | — | — | 2 | 0 |
| | CH₂CH₂COOCH₃ | | | | | | | | | |
| 456 | N—CH₂CH₂COO— | CF₃—C—CH₃ | NCH₃ | H | H | H | — | — | 2 | 0 |
| | (Cypropyl) | | | | | | | | | |
| 457 | N— | CF₃—C—CH₃ | NCH₃ | H | H | H | — | — | 2 | 0 |
| | CH₂CH₂COOCH₂Ph | | | | | | | | | |
| 458 | N— | CF₃—C—CH₃ | NCH₃ | H | H | H | — | — | 2 | 0 |
| | CH₂CH₂COSCH₃ | | | | | | | | | |
| 459 | N— | CF₃—C—CH₃ | NCH₃ | H | H | H | — | — | 2 | 0 |
| | CH₂CH₂CONHCH₃ | | | | | | | | | |
| 460 | N—CH₂OCOCH₃ | CF₃—C—CH₃ | NCH₃ | H | H | H | | — | 2 | 0 |
| 461 | N—CH₂OCO- | CF₃—C—CH₃ | NCH₃ | H | H | H | — | — | 2 | 0 |
| | (Cypentyl) | | | | | | | | | |
| 462 | N— | CF₃—C—CH₃ | NCH₃ | H | H | H | — | — | 2 | 0 |
| | CH₂OCO(Benzyl) | | | | | | | | | |
| 463 | N— | CF₃—C—CH₃ | NCH₃ | H | H | H | — | — | 2 | 0 |
| | CH₂OCH(CH₃)₂ | | | | | | | | | |
| 464 | N—CH₂O- | CF₃—C—CH₃ | NCH₃ | H | H | H | — | — | 2 | 0 |
| | (Benzyl) | | | | | | | | | |
| 465 | N— | CF₃—C—CH₃ | NCH₃ | H | H | H | — | — | 2 | 0 |
| | CH₂SCH(CH₃)₂ | | | | | | | | | |
| 466 | N—CH₂SCOCH₃ | CF₃—C—CH₃ | NCH₃ | H | H | H | — | — | 2 | 0 |
| 467 | N— | CF₃—C—CH₃ | NCH₃ | H | H | H | — | — | 2 | 0 |
| | CH₂SCOCypentyl | | | | | | | | | |
| 468 | N—COCH₃ | CH₂ | S | H | H | H | CH₃ | CH₃ | 2 | 1 |
| 469 | N—COCH₃ | CH₂ | S | H | CH₃ | CH₃ | CH₃ | CH₃ | 2 | 1 |
| 470 | N—COCH₃ | CH₂ | S(O) | H | H | H | CH₃ | CH₃ | 2 | 1 |
| 471 | N—COCH₃ | CH₂ | S(O) | H | CH₃ | CH₃ | CH₃ | CH₃ | 2 | 1 |
| 472 | N—COCH₃ | CH₂ | S(O)₂ | H | H | H | CH₃ | CH₃ | 2 | 1 |
| 473 | N—COCH₃ | CH₂ | S(O)₂ | H | CH₃ | CH₃ | CH₃ | CH₃ | 2 | 1 |
| 474 | N—COCH₃ | CH₂ | CH₂ | H | H | H | CH₃ | CH₃ | 2 | 1 |
| 475 | N—COCH₃ | CH₂ | CH₂ | H | CH₃ | CH₃ | CH₃ | CH₃ | 2 | 1 |
| 476 | N—COCH₃ | CH₂ | CH₂ | H | H | H | H | H | 2 | 1 |
| 477 | N—COOCH₃ | CH₂ | S | H | H | H | CH₃ | CH₃ | 2 | 1 |
| 478 | N—COOCH₃ | CH₂ | S | H | CH₃ | CH₃ | CH₃ | CH₃ | 2 | 1 |
| 479 | N—COOCH₃ | CH₂ | S(O) | H | H | H | CH₃ | CH₃ | 2 | 1 |
| 480 | N—COOCH₃ | CH₂ | S(O) | H | CH₃ | CH₃ | CH₃ | CH₃ | 2 | 1 |
| 481 | N—COOCH₃ | CH₂ | S(O)₂ | H | H | H | CH₃ | CH₃ | 2 | 1 |
| 482 | N—COOCH₃ | CH₂ | S(O)₂ | H | CH₃ | CH₃ | CH₃ | CH₃ | 2 | 1 |
| 483 | N—COOCH₃ | CH₂ | CH₂ | H | H | H | CH₃ | CH₃ | 2 | 1 |
| 484 | N—COOCH₃ | CH₂ | CH₂ | F | CH₃ | CH₃ | CH₃ | CH₃ | 2 | 1 |
| 485 | N—COOCH₃ | CH₂ | CH₂ | F | H | H | H | H | 2 | 1 |
| 486 | N—COCH₃ | CHCH₃ | S | H | H | H | CH₃ | CH₃ | 2 | 1 |
| 487 | N—COCH₃ | CHCH₃ | S | H | CH₃ | CH₃ | CH₃ | CH₃ | 2 | 1 |
| 488 | N—COCH₃ | CHCH₃ | S(O) | H | H | H | CH₃ | CH₃ | 2 | 1 |
| 489 | N—COCH₃ | CHCH₃ | S(O) | H | CH₃ | CH₃ | CH₃ | CH₃ | 2 | 1 |
| 490 | N—COCH₃ | CHCH₃ | S(O)₂ | H | H | H | CH₃ | CH₃ | 2 | 1 |
| 491 | N—COCH₃ | CHCH₃ | S(O)₂ | H | CH₃ | CH₃ | CH₃ | CH₃ | 2 | 1 |
| 492 | N—COCH₃ | CHCH₃ | CH₂ | H | H | H | CH₃ | CH₃ | 2 | 1 |
| 493 | N—COCH₃ | CHCH₃ | CH₂ | H | CH₃ | CH₃ | CH₃ | CH₃ | 2 | 1 |
| 494 | N—COCH₃ | CHCH₃ | CH₂ | H | H | H | H | H | 2 | 1 |
| 495 | N—COOCH₃ | CHCH₃ | S | H | H | H | CH₃ | CH₃ | 2 | 1 |
| 496 | N—COOCH₃ | CHCH₃ | S | H | CH₃ | CH₃ | CH₃ | CH₃ | 2 | 1 |
| 497 | N—COOCH₃ | CHCH₃ | S(O) | H | H | H | CH₃ | CH₃ | 2 | 1 |
| 498 | N—COOCH₃ | CHCH₃ | S(O) | H | CH₃ | CH₃ | CH₃ | CH₃ | 2 | 1 |
| 499 | N—COOCH₃ | CHCH₃ | S(O)₂ | H | H | H | CH₃ | CH₃ | 2 | 1 |
| 500 | N—COOCH₃ | CHCH₃ | S(O)₂ | H | CH₃ | CH₃ | CH₃ | CH₃ | 2 | 1 |
| 501 | N—COOCH₃ | CHCH₃ | CH₂ | H | H | H | CH₃ | CH₃ | 2 | 1 |
| 502 | N—COOCH₃ | CHCH₃ | CH₂ | H | CH₃ | CH₃ | CH₃ | CH₃ | 2 | 1 |
| 503 | N—COOCH₃ | CHCH₃ | CH₂ | H | H | H | H | H | 2 | 1 |
| 504 | N—COCH₃ | C(CH₃)₂ | S | H | H | H | CH₃ | CH₃ | 2 | 1 |
| 505 | N—COCH₃ | C(CH₃)₂ | S | H | CH₃ | CH₃ | CH₃ | CH₃ | 2 | 1 |
| 506 | N—COCH₃ | C(CH₃)₂ | S(O) | H | H | H | CH₃ | CH₃ | 2 | 1 |
| 507 | N—COCH₃ | C(CH₃)₂ | S(O) | H | CH₃ | CH₃ | CH₃ | CH₃ | 2 | 1 |
| 508 | N—COCH₃ | C(CH₃)₂ | S(O)₂ | H | H | H | CH₃ | CH₃ | 2 | 1 |
| 509 | N—COCH₃ | C(CH₃)₂ | S(O)₂ | H | CH₃ | CH₃ | CH₃ | CH₃ | 2 | 1 |
| 510 | N—COCH₃ | C(CH₃)₂ | CH₂ | H | H | H | CH₃ | CH₃ | 2 | 1 |
| 511 | N—COCH₃ | C(CH₃)₂ | CH₂ | H | CH₃ | CH₃ | CH₃ | CH₃ | 2 | 1 |
| 512 | N—COCH₃ | C(CH₃)₂ | CH₂ | H | H | H | H | H | 2 | 1 |
| 513 | N—COOCH₃ | C(CH₃)₂ | S | H | H | H | CH₃ | CH₃ | 2 | 1 |
| 514 | N—COOCH₃ | C(CH₃)₂ | S | H | CH₃ | CH₃ | CH₃ | CH₃ | 2 | 1 |
| 515 | N—COOCH₃ | C(CH₃)₂ | S(O) | H | H | H | CH₃ | CH₃ | 2 | 1 |
| 516 | N—COOCH₃ | C(CH₃)₂ | S(O) | H | CH₃ | CH₃ | CH₃ | CH₃ | 2 | 1 |
| 517 | N—COOCH₃ | C(CH₃)₂ | S(O)₂ | H | H | H | CH₃ | CH₃ | 2 | 1 |
| 518 | N—COOCH₃ | C(CH₃)₂ | S(O)₂ | H | CH₃ | CH₃ | CH₃ | CH₃ | 2 | 1 |
| 519 | N—COOCH₃ | C(CH₃)₂ | CH₂ | H | H | H | CH₃ | CH₃ | 2 | 1 |
| 520 | N—COOCH₃ | C(CH₃)₂ | CH₂ | H | CH₃ | CH₃ | CH₃ | CH₃ | 2 | 1 |
| 521 | N—COOCH₃ | C(CH₃)₂ | CH₂ | H | H | H | H | H | 2 | 1 |
| 522 | N—COCH₃ | C═S | S | F | H | H | — | — | 2 | 0 |
| 523 | N—COCH₃ | C═S | S(O) | F | H | H | — | — | 2 | 0 |
| 524 | N—COCH₃ | C═S | S(O)₂ | F | H | H | — | — | 2 | 0 |
| 525 | N—COCH₃ | C═O | S(O) | F | H | H | — | — | 2 | 0 |
| 526 | N—COCH₃ | C═O | S(O)₂ | F | H | H | — | — | 2 | 0 |
| 527 | N—COC(CH₃)₃ | C═O | S | F | H | H | — | — | 2 | 0 |
| 528 | N— | C═O | S | F | H | H | — | — | 2 | 0 |
| | COCH₂CH(CH₃)₂ | | | | | | | | | |
| 529 | N—CO- | C═O | S | F | H | H | — | — | 2 | 0 |
| | Cypentyl) | | | | | | | | | |
| 530 | N—COButyl | C═O | S | F | H | H | — | — | 2 | 0 |
| 531 | N—COOC(CH₃)₃ | C═O | O | F | H | H | — | — | 2 | 0 |
| 532 | N—COCH₃ | C═O | O | F | H | H | — | — | 2 | 0 |
| 533 | N—COCH₂CH₃ | C═O | O | F | H | H | — | — | 2 | 0 |
| 534 | N—COCH(CH₃)₂ | C═O | O | F | H | H | — | — | 2 | 0 |
| 535 | N—COCH(CH₃)₂ | C═O | S | F | H | H | — | — | 2 | 0 |
| 536 | N—COCH₂CH₃ | C═O | S | F | H | H | — | — | 2 | 0 |
| 537 | N—COSCH(CH₃)₂ | C═O | S | F | H | H | — | — | 2 | 0 |
| 538 | N—COCH₃ | C═O | S | F | H | H | H | H | 2 | 1 |
| 539 | N—COCH₃ | C═O | S(O)2 | F | H | H | — | — | 2 | 0 |
| 540 | N—COCH₃ | C═O | S(O) | F | H | H | — | — | 2 | 0 |
| 541 | N—CH₂COCH₃ | C═O | S | F | H | H | — | — | 2 | 0 |
| 542 | N—CH₂CO- | C═O | S | F | H | H | — | — | 2 | 0 |
| | (Cypentyl) | | | | | | | | | |
| 543 | N—CH₂CO-(4-F)-Ph | C═O | S | F | H | H | — | — | 2 | 0 |
| 544 | N—CH₂CO-2- | C═O | S | F | H | H | — | — | 2 | 0 |
| | Thiazolyl | | | | | | | | | |
| 545 | N— | C═O | S | F | H | H | — | — | 2 | 0 |
| | CH₂CH₂COOCH₃ | | | | | | | | | |
| 546 | N—CH₂CH₂COO- | C═O | S | F | H | H | — | — | 2 | 0 |
| | (Cypropyl) | | | | | | | | | |
| 547 | N— | C═O | S | F | H | H | — | — | 2 | 0 |
| | CH₂CH₂COOCH₂Ph | | | | | | | | | |
| 548 | N— | C═O | S | F | H | H | — | — | 2 | 0 |
| | CH₂CH₂COSCH₃ | | | | | | | | | |
| 549 | N— | C═O | S | F | H | H | — | — | 2 | 0 |
| | CH₂CH₂CONHCH₃ | | | | | | | | | |
| 550 | N—CH₂OCOCH₃ | C═O | S | F | H | H | — | — | 2 | 0 |
| 551 | N— | C═O | S | F | H | H | — | — | 2 | 0 |
| | CH₂OCO(Cypentyl) | | | | | | | | | |
| 552 | N— | C═O | S | F | H | H | — | — | 2 | 0 |
| | CH₂OCO(Benzyl) | | | | | | | | | |
| 553 | N— | C═O | S | F | H | H | — | — | 2 | 0 |
| | CH₂OCH(CH₃)₂ | | | | | | | | | |
| 554 | N— | C═O | S | F | H | H | — | — | 2 | 0 |
| | CH₂O(Benzyl) | | | | | | | | | |
| 555 | N— | C═O | S | F | H | H | — | — | 2 | 0 |
| | CH₂SCH(CH₃)₂ | | | | | | | | | |
| 556 | N—CH₂SCOCH₃ | C═O | S | F | H | H | — | — | 2 | 0 |
| 557 | N— | C═O | S | F | H | H | — | — | 2 | 0 |
| | CH₂SCO(Cypentyl) | | | | | | | | | |
| 558 | N—COC(CH₃)₃ | C═S | S | F | H | H | — | — | 2 | 0 |
| 559 | N— | C═S | S | F | H | H | — | — | 2 | 0 |
| | COCH₂CH(CH₃)₂ | | | | | | | | | |
| 560 | N—CO- | C═S | S | F | H | H | — | — | 2 | 0 |
| | (Cypentyl) | | | | | | | | | |
| 561 | N—COOC(CH₃)₃ | C═S | O | F | H | H | — | — | 2 | 0 |
| 562 | N—COCH₃ | C═S | O | F | H | H | — | — | 2 | 0 |
| 563 | N—COCH₂CH₃ | C═S | O | F | H | H | — | — | 2 | 0 |
| 564 | N—COCH₂CH₃ | C═S | O | F | H | H | — | — | 2 | 0 |
| 565 | N—COCH₃ | C═S | S(O)₂ | F | H | H | — | — | 2 | 0 |
| 566 | N—COCH₂CH₃ | C═S | S(O)₂ | F | H | H | — | — | 2 | 0 |
| 567 | N—COSCH(CH₃)₂ | C═S | S | F | H | H | — | — | 2 | 0 |
| 568 | N—COCH₃ | C═S | S | F | H | H | H | H | 2 | 1 |
| 569 | N—COCH(CH₃)₂ | C═S | S | F | H | H | — | — | 2 | 0 |
| 570 | N—COCH₃ | C═S | S(O) | F | H | H | — | — | 2 | 0 |
| 571 | N—CH₂COCH₃ | C═S | S | F | H | H | — | — | 2 | 0 |
| 572 | N—CH₂CO- | C═S | S | F | H | H | — | — | 2 | 0 |
| | (Cypentyl) | | | | | | | | | |
| 573 | N—CH₂CO-(4-F)-Ph | C═S | S | F | H | H | — | — | 2 | 0 |
| 574 | N—CH₂CO-2- | C═S | S | F | H | H | — | — | 2 | 0 |
| | Thiazolyl | | | | | | | | | |
| 575 | N— | C═S | S | F | H | H | — | — | 2 | 0 |
| | CH₂CH₂COOCH₃ | | | | | | | | | |
| 576 | N—CH₂CH₂COO- | C═S | S | F | H | H | — | — | 2 | 0 |
| | (Cypropyl) | | | | | | | | | |
| 577 | N— | C═S | S | F | H | H | — | — | 2 | 0 |
| | CH₂CH₂COOCH₂Ph | | | | | | | | | |
| 578 | N— | C═S | S | F | H | H | — | — | 2 | 0 |
| | CH₂CH₂COSCH₃ | | | | | | | | | |
| 579 | N— | C═S | S | F | H | H | — | — | 2 | 0 |
| | CH₂CH₂CONHCH₃ | | | | | | | | | |
| 580 | N—CH₂OCOCH₃ | C═S | S | F | H | H | — | — | 2 | 0 |
| 581 | N— | C═S | S | F | H | H | — | — | 2 | 0 |
| | CH₂OCO(Cypentyl) | | | | | | | | | |
| 582 | N— | C═S | S | F | H | H | — | — | 2 | 0 |
| | CH₂OCOBenzyl | | | | | | | | | |
| 583 | N— | C═S | S | F | H | H | — | — | 2 | 0 |
| | CH₂OCH(CH₃)₂ | | | | | | | | | |
| 584 | N—CH₂OBenzyl | C═S | S | F | H | H | — | — | 2 | 0 |
| 585 | N— | C═S | S | F | H | H | — | — | 2 | 0 |
| | CH₂SCH(CH₃)₂ | | | | | | | | | |
| 586 | N—CH₂SCOCH₃ | C═S | S | F | H | H | — | — | 2 | 0 |
| 587 | N— | C═S | S | F | H | H | — | — | 2 | 0 |
| | CH₂SCO(Cypentyl) | | | | | | | | | |
| 588 | N—COCH₃ | C═O | S | H | H | H | H | H | 2 | 1 |
| 589 | N—COCH₃ | C═O | S | H | H | H | CH₃ | CH₃ | 2 | 1 |
| 589 | N—COCH₃ | C═O | S | H | CH₃ | CH₃ | CH₃ | CH₃ | 2 | 1 |
| 590 | N—COCH₃ | C═O | S(O) | H | H | H | CH₃ | CH₃ | 2 | 1 |
| 591 | N—COCH₃ | C═O | S(O) | H | CH₃ | CH₃ | CH₃ | CH₃ | 2 | 1 |
| 592 | N—COCH₃ | C═S | S(O)₂ | H | CH₃ | CH₃ | CH₃ | CH₃ | 2 | 1 |
| 593 | N—COCH₃ | C═O | CH₂ | H | H | H | H | H | 2 | 1 |
| 594 | N—COCH₃ | C═O | CH₂ | H | H | H | CH₃ | CH₃ | 2 | 1 |
| 595 | N—COOCH₃ | C═O | CH₂ | H | CH₃ | CH₃ | CH₃ | CH₃ | 2 | 1 |
| 596 | N—COOCH₃ | C═S | CH₂ | H | H | H | CH₃ | CH₃ | 2 | 1 |
| 597 | N—COCH₃ | C═O | CH₂ | H | CH₃ | CH₃ | CH₃ | CH₃ | 2 | 1 |
| 598 | N—COCH₃ | C═S | CH₂ | H | H | H | CH₃ | CH₃ | 2 | 1 |
| 599 | N—COCH₃ | C═S | CH₂ | H | CH₃ | CH₃ | CH₃ | CH₃ | 2 | 1 |
| 600 | N—COCH₃ | CH- | O | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 601 | N—COCH₃ | CH- | S | F | H | H | — | — | 2 | 0 |
| | | (1S,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 602 | N—COCH₃ | CH- | S | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 603 | N—COCH₃ | CH- | S | F | H | H | — | — | 2 | 0 |
| | | (1R,2S,3S,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 604 | N—COCH₃ | CH- | O | F | H | H | — | — | 2 | 0 |
| | | (1S,2S,3R,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 605 | N—COCH₃ | CH_(1R,2R,3R)- | S | F | H | H | — | — | 2 | 0 |
| | | (1,2,3,4)- | | | | | | | | |
| | | (tetra- | | | | | | | | |
| | | acetoxy-butyl | | | | | | | | |
| 606 | N—COCH₃ | CH_(1R,2S,3R, | O | F | H | H | — | — | 2 | 0 |
| | | 4R)-- | | | | | | | | |
| | | (1,2,3,4,5)- | | | | | | | | |
| | | (penta- | | | | | | | | |
| | | methoxy- | | | | | | | | |
| | | pentyl | | | | | | | | |
| 607 | N—COCH₃ | CH- | O | F | H | H | — | — | 2 | 0 |
| | | (1R,2S,3S,4R)-- | | | | | | | | |
| | | (1,2,3,4,5)- | | | | | | | | |
| | | (penta- | | | | | | | | |
| | | acetoxy- | | | | | | | | |
| | | pentyl | | | | | | | | |
| 608 | N—COCH₃ | CH_(1R, 2S, 3R, | O | F | H | H | — | — | 2 | 0 |
| | | 4R)-- | | | | | | | | |
| | | (1,2,3,4,5)- | | | | | | | | |
| | | (penta- | | | | | | | | |
| | | hydroxy- | | | | | | | | |
| | | pentyl | | | | | | | | |
| 609 | N—COCH₃ | CH- | S | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | hydroxy- | | | | | | | | |
| | | pentyl | | | | | | | | |
| 610 | N—COCH₃ | CH- | S | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | ethoxy- | | | | | | | | |
| | | pentyl | | | | | | | | |
| 611 | N—COCH₃ | CH- | S | F | H | H | — | — | 2 | 0 |
| | | (1R, 2S, 3S, 4R)-- | | | | | | | | |
| | | (1,2,3,4,5)- | | | | | | | | |
| | | (penta- | | | | | | | | |
| | | hydroxy- | | | | | | | | |
| | | pentyl | | | | | | | | |
| 612 | N—COCH₃ | CH- | O | F | H | H | — | — | 2 | 0 |
| | | (1S,2S,3R,4R)-- | | | | | | | | |
| | | (1,2,3,4,5)- | | | | | | | | |
| | | (penta | | | | | | | | |
| | | hydroxy- | | | | | | | | |
| | | pentyl | | | | | | | | |
| 613 | N—COCH₃ | CH- | O | F | H | H | — | — | 2 | 0 |
| | | (1R,2S,3R,4R)-- | | | | | | | | |
| | | (1,2,3,4,5)- | | | | | | | | |
| | | (penta- | | | | | | | | |
| | | ethoxy- | | | | | | | | |
| | | pentyl | | | | | | | | |
| 614 | N—COCH₃ | CH- | O | F | H | H | — | — | 2 | 0 |
| | | (1R,2S,3S,4R)-- | | | | | | | | |
| | | (1,2,3,4,5)- | | | | | | | | |
| | | (penta- | | | | | | | | |
| | | hydroxy- | | | | | | | | |
| | | pentyl | | | | | | | | |
| 615 | N—CH₃ | CH₂ | O | F | H | H | — | — | 2 | 0 |
| 616 | N—CH(CH₃)₂ | CH₂ | O | F | H | H | — | — | 2 | 0 |
| 617 | N-Cypropyl | CH₂ | O | F | H | H | — | — | 2 | 0 |
| 618 | N—CH₂-Cypropyl | CH₂ | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 621 | N-H | CH₂ | CH₂ | F | H | H | — | — | 2 | 0 |
| 622 | N—COOC(CH₃)₃ | CH₂ | CH₂ | F | H | H | — | — | 2 | 0 |
| 623 | N—CH₃ | CH₂ | CH₂ | F | H | H | — | — | 2 | 0 |
| 624 | N—COCH₃ | CH₂ | CH₂ | F | H | H | — | — | 2 | 0 |
| 625 | N—COCF₃ | CH₂ | CH₂ | F | H | H | — | — | 2 | 0 |
| 626 | N—CH₃S(O)₂ | CH₂ | CH₂ | F | H | H | — | — | 2 | 0 |
| 627 | N—CF₃S(O)₂ | CH₂ | CH₂ | F | H | H | — | — | 2 | 0 |
| 628 | N—COButyl | CH₂ | CH₂ | F | H | H | — | — | 2 | 0 |
| 629 | N—CO-(2,4- | CH₂ | CH₂ | F | H | H | — | — | 2 | 0 |
| | diF)-Ph | | | | | | | | | |
| 630 | N—CO-(4- | CH₂ | CH₂ | F | H | H | — | — | 2 | 0 |
| | OCF₃)-Ph | | | | | | | | | |
| 631 | N—CO-(2-F, | CH₂ | CH₂ | F | H | H | — | — | 2 | 0 |
| | 6-CF₃)-Ph | | | | | | | | | |
| 632 | N— | CH₂ | CH₂ | F | H | H | — | — | 2 | 0 |
| | COOCH₂CH(CH₃)₂ | | | | | | | | | |
| 633 | N—COOCC(CH₃)₂ | CH₂ | CH₂ | F | H | H | — | — | 2 | 0 |
| 634 | N—COPh | CH₂ | CH₂ | F | H | H | — | — | 2 | 0 |
| 635 | N—CO(4-CF₃)-Ph | CH₂ | CH₂ | F | H | H | — | — | 2 | 0 |
| 636 | N—CO(3-CF₃)- | CH₂ | CH₂ | F | H | H | — | — | 2 | 0 |
| | Ph | | | | | | | | | |
| 637 | N—CO-(2,3,6- | CH₂ | CH₂ | F | H | H | — | — | 2 | 0 |
| | tri-F, -5-5--OCH₃-)-Ph | | | | | | | | | |
| 638 | N—COEthyl | CH₂ | CH₂ | F | H | H | — | — | 2 | 0 |
| 639 | N—CO-n- | CH₂ | CH₂ | F | H | H | — | — | 2 | 0 |
| | Propyl | | | | | | | | | |
| 640 | N—CO-i- | CH₂ | CH₂ | F | H | H | — | — | 2 | 0 |
| | Propyl | | | | | | | | | |
| 641 | N—COOCH₃ | CH₂ | CH₂ | F | H | H | — | — | 2 | 0 |
| 642 | N—COO-i- | CH₂ | CH₂ | F | H | H | — | — | 2 | 0 |
| | Propyl | | | | | | | | | |
| 643 | N—COO-i- | CH₂ | CH₂ | F | H | H | — | — | 2 | 0 |
| | Butyl | | | | | | | | | |
| 644 | N—COOCH₃ | CH₂ | CH₂ | F | H | H | — | — | 2 | 0 |
| 645 | N— | CH₂ | CH₂ | F | H | H | — | — | 2 | 0 |
| | P(═O) (OEthyl)₂ | | | | | | | | | |
| 646 | N— | CH₂ | CH₂ | F | H | H | — | — | 2 | 0 |
| | P(═O) (OPh)₂ | | | | | | | | | |
| 647 | N— | CH₂ | CH₂ | F | H | H | — | — | 2 | 0 |
| | P(═S)(OCH₃)₂ | | | | | | | | | |
| 648 | N-i-propyl | CH₂ | CH₂ | F | H | H | — | — | 2 | 0 |
| 649 | N-t-Butyl | CH₂ | CH₂ | F | H | H | — | — | 2 | 0 |
| 650 | 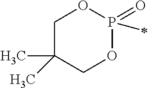 | CH₂ | CH₂ | F | H | H | — | — | 2 | 0 |
| 651 | 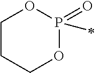 | CH₂ | CH₂ | F | H | H | — | — | 2 | 0 |
| 652 | 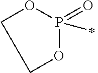 | CH₂ | CH₂ | F | H | H | — | — | 2 | 0 |
| 653 | N—H | CH₂ | CH₂ | H | H | H | — | — | 2 | 0 |
| 654 | N—COOC(CH₃)₃ | CH₂ | CH₂ | H | H | H | — | — | 2 | 0 |
| 655 | N—CH₃ | CH₂ | CH₂ | H | H | H | — | — | 2 | 0 |
| 656 | N—COCH₃ | CH₂ | CH₂ | H | H | H | — | — | 2 | 0 |
| 657 | N—COCF₃ | CH₂ | CH₂ | H | H | H | — | — | 2 | 0 |
| 658 | N—CH₃S(O)₂ | CH₂ | CH₂ | H | H | H | — | — | 2 | 0 |
| 659 | N—CF₃S(O)₂ | CH₂ | CH₂ | H | H | H | — | — | 2 | 0 |
| 660 | N—COButyl | CH₂ | CH₂ | H | H | H | — | — | 2 | 0 |
| 661 | N—CO-(2,4- | CH₂ | CH₂ | H | H | H | — | — | 2 | 0 |
| | diF)-Ph | | | | | | | | | |
| 662 | N—CO-(4- | CH₂ | CH₂ | H | H | H | — | — | 2 | 0 |
| | OCF₃)-Ph | | | | | | | | | |
| 663 | N—CO-(2-F, | CH₂ | CH₂ | H | H | H | — | — | 2 | 0 |
| | 6-CF₃)-Ph | | | | | | | | | |
| 664 | N—H | CH₂ | CH₂ | F | H | H | — | — | 4 | 0 |
| 665 | N—COOC(CH₃)₃ | CH₂ | CH₂ | F | H | H | — | — | 4 | 0 |
| 666 | N—CH₃ | CH₂ | CH₂ | F | H | H | — | — | 4 | 0 |
| 667 | N—COCH₃ | CH₂ | CH₂ | F | H | H | — | — | 4 | 0 |
| 668 | N—COCF₃ | CH₂ | CH₂ | F | H | H | — | — | 4 | 0 |
| 669 | N—CH₃S(O)₂ | CH₂ | CH₂ | F | H | H | — | — | 4 | 0 |
| 670 | N—CF₃S(O)₂ | CH₂ | CH₂ | F | H | H | — | — | 4 | 0 |
| 671 | N—COButyl | CH₂ | CH₂ | F | H | H | — | — | 4 | 0 |
| 672 | N—CO-(2,4- | CH₂ | CH₂ | F | H | H | — | — | 4 | 0 |
| | diF)-Ph | | | | | | | | | |
| 673 | N—CO-(4- | CH₂ | CH₂ | F | H | H | — | — | 4 | 0 |
| | OCF₃)-Ph | | | | | | | | | |
| 674 | N—CO-(2-F, | CH₂ | CH₂ | F | H | H | — | — | 4 | 0 |
| | 6-CF₃)-Ph | | | | | | | | | |
| 675 | N—COCH₃ | C═O | CH₂ | F | H | H | H | H | 2 | 1 |
| 676 | S | CH₂ | CH₂ | F | H | H | H | H | 2 | 1 |
| 677 | O | CH₂ | CH₂ | F | H | H | H | H | 2 | 1 |
| 678 | S | CH₂ | CH₂ | F | H | H | — | — | 2 | 0 |
| 679 | O | CH₂ | CH₂ | F | H | H | — | — | 2 | 0 |

Particularly preferred are compounds having general formula (I) wherein E, Z, Y, R₃, R₄, R₅, R₆, X, n and m have the meanings indicated in Table 2.

TABLE 2

| Comp | | | | | | | | | | |
|---|---|---|---|---|---|---|---|---|---|---|
| Nr. | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO-Cypropyl | CH₂ | S | F | H | H | — | — | 2 | 0 |
| 7 | N—COCH₃ | C—(CH₃)₂ | S | F | CH₃ | CH₃ | — | — | 2 | 0 |
| 8 | N—COCH₃ | CH₃—C—COOCH₃ | S | F | H | H | — | — | 2 | 0 |
| 9 | N—COCH₃ | CH- | S | F | H | H | — | — | 2 | 0 |
| | | (1R,2S,3R,4R)-- | | | | | | | | |
| | | (1,2,3,4,5)-(penta | | | | | | | | |
| | | Acetoxy)-pentyl | | | | | | | | |
| 10 | N—COCH₃ | CH—(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH₃)₂ | CH₂ | S | F | H | H | — | — | 2 | 0 |
| 13 | N—CO-Cypentyl | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 16 | N—COCH₃ | CF₃—C—CH₃ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 22 | N—COCH₃ | CH₂ | S | F | H | H | H | H | 2 | 1 |
| 23 | N—COCH₃ | CH₂ | S | F | H | H | — | — | 2 | 0 |
| 25 | N—COCH₃ | CH₂ | S(O) | F | H | H | — | — | 2 | 0 |
| 26 | N—CH₂COCH₃ | CH₂ | S | F | H | H | — | — | 2 | 0 |
| 30 | N—H₂CH₂COOCH₃ | CH₂ | S | F | H | H | — | — | 2 | 0 |
| 35 | N—CH₂OCOCH₃ | CH₂ | S | F | H | H | — | — | 2 | 0 |
| 38 | N—H₂OCH(CH₃)₂ | CH₂ | S | F | H | H | — | — | 2 | 0 |
| 39 | N—CH₂OBenzyl | CH₂ | S | F | H | H | — | — | 2 | 0 |
| 44 | N—COCH₃ | CH(CH₃)₂ | S(O)₂ | F | H | H | — | — | 2 | 0 |
| 45 | N—CH₂COCH₃ | CH(CH₃)₂ | S | F | H | H | — | — | 2 | 0 |
| 54 | N—CH₂OCOCH₃ | CH(CH₃)₂ | S | F | H | H | — | — | 2 | 0 |
| 58 | N—CH₂OBenzyl | CH(CH₃)₂ | S | F | H | H | — | — | 2 | 0 |
| 64 | N—CH₂COCH₃ | CF₃—C—CH₃ | O | F | H | H | — | — | 2 | 0 |
| 68 | N—H₂CH₂COOCH₃ | CF₃—C—CH₃ | O | F | H | H | — | — | 2 | 0 |
| 73 | N—CH₂OCOCH₃ | CF₃—C—CH₃ | O | F | H | H | — | — | 2 | 0 |
| 77 | N—CH₂OBenzyl | CF₃—C—CH₃ | O | F | H | H | — | — | 2 | 0 |
| 81 | N—COCH₃ | CH₂ | NH | F | H | H | — | — | 2 | 0 |
| 84 | N—CH₂COCH₃ | CF₃—C—CH₃ | NH | F | H | H | — | — | 2 | 0 |
| 93 | N—CH₂OCOCH₃ | CF₃—C—CH₃ | NH | F | H | H | — | — | 2 | 0 |
| 101 | N—COCH₃ | CH₂ | NCH₃ | F | H | H | — | — | 2 | 0 |
| 104 | N—CH₂COCH₃ | CF₃—C—CH₃ | NCH₃ | F | H | H | — | — | 2 | 0 |
| 113 | N—CH₂OCOCH₃ | CF₃—C—CH₃ | NCH₃ | F | H | H | — | — | 2 | 0 |
| 121 | N—COCH₃ | CH₂ | S | F | H | H | CH₃ | CH₃ | 2 | 1 |
| 123 | N—COCH₃ | CH₂ | S | F | CH₃ | CH₃ | CH₃ | CH₃ | 2 | 1 |
| 126 | N—COCH₃ | CH₂ | S(O)₂ | F | H | H | CH₃ | CH₃ | 2 | 1 |
| 127 | N—COCH₃ | CH₂ | S(O)₂ | F | CH₃ | CH₃ | CH₃ | CH₃ | 2 | 1 |
| 128 | N—COCH₃ | CH₂ | CH₂ | F | H | H | CH₃ | CH₃ | 2 | 1 |
| 129 | N—COCH₃ | CH₂ | CH₂ | F | CH₃ | CH₃ | CH₃ | CH₃ | 2 | 1 |
| 130 | N—COCH₃ | CH₂ | CH₂ | F | H | H | H | H | 2 | 1 |
| 131 | N—COOCH₃ | CH₂ | S | F | H | H | CH₃ | CH₃ | 2 | 1 |
| 132 | N—COOCH₃ | CH₂ | S | F | CH₃ | CH₃ | CH₃ | CH₃ | 2 | 1 |
| 135 | N—COOCH₃ | CH₂ | S(O)₂ | F | H | H | CH₃ | CH₃ | 2 | 1 |
| 136 | N—COOCH₃ | CH₂ | S(O)₂ | F | CH₃ | CH₃ | CH₃ | CH₃ | 2 | 1 |
| 137 | N—COOCH₃ | CH₂ | CH₂ | F | H | H | CH₃ | CH₃ | 2 | 1 |
| 138 | N—COOCH₃ | CH₂ | CH₂ | F | CH₃ | CH₃ | CH₃ | CH₃ | 2 | 1 |
| 139 | N—COOCH₃ | CH₂ | CH₂ | F | H | H | H | H | 2 | 1 |
| 140 | N—COCH₃ | CHCH₃ | S | F | H | H | CH₃ | CH₃ | 2 | 1 |
| 141 | N—COCH₃ | CHCH₃ | S | F | CH₃ | CH₃ | CH₃ | CH₃ | 2 | 1 |
| 144 | N—COCH₃ | CHCH₃ | S(O)₂ | F | H | H | CH₃ | CH₃ | 2 | 1 |
| 145 | N—COCH₃ | CHCH₃ | S(O)₂ | F | CH₃ | CH₃ | CH₃ | CH₃ | 2 | 1 |
| 146 | N—COCH₃ | CHCH₃ | CH₂ | F | H | H | CH₃ | CH₃ | 2 | 1 |
| 147 | N—COCH₃ | CHCH₃ | CH₂ | F | CH₃ | CH₃ | CH₃ | CH₃ | 2 | 1 |
| 148 | N—COCH₃ | CHCH₃ | CH₂ | F | H | H | H | H | 2 | 1 |
| 149 | N—COOCH₃ | CHCH₃ | S | F | H | H | CH₃ | CH₃ | 2 | 1 |
| 150 | N—COOCH₃ | CHCH₃ | S | F | CH₃ | CH₃ | CH₃ | CH₃ | 2 | 1 |
| 153 | N—COOCH₃ | CHCH₃ | S(O)₂ | F | H | H | CH₃ | CH₃ | 2 | 1 |
| 154 | N—COOCH₃ | CHCH₃ | S(O)₂ | F | CH₃ | CH₃ | CH₃ | CH₃ | 2 | 1 |
| 155 | N—COOCH₃ | CHCH₃ | CH₂ | F | H | H | CH₃ | CH₃ | 2 | 1 |
| 156 | N—COOCH₃ | CHCH₃ | CH₂ | F | CH₃ | CH₃ | CH₃ | CH₃ | 2 | 1 |
| 157 | N—COOCH₃ | CHCH₃ | CH₂ | F | H | H | H | H | 2 | 1 |
| 158 | N—COCH₃ | C(CH₃)₂ | S | F | H | H | CH₃ | CH₃ | 2 | 1 |
| 159 | N—COCH₃ | C(CH₃)₂ | S | F | CH₃ | CH₃ | CH₃ | CH₃ | 2 | 1 |
| 162 | N—COCH₃ | C(CH₃)₂ | S(O)₂ | F | H | H | CH₃ | CH₃ | 2 | 1 |
| 163 | N—COCH₃ | C(CH₃)₂ | S(O)₂ | F | CH₃ | CH₃ | CH₃ | CH₃ | 2 | 1 |
| 164 | N—COCH₃ | C(CH₃)₂ | CH₂ | F | H | H | CH₃ | CH₃ | 2 | 1 |
| 165 | N—COCH₃ | C(CH₃)₂ | CH₂ | F | CH₃ | CH₃ | CH₃ | CH₃ | 2 | 1 |
| 166 | N—COCH₃ | C(CH₃)₂ | CH₂ | F | H | H | H | H | 2 | 1 |
| 167 | N—COOCH₃ | C(CH₃)₂ | S | F | H | H | CH₃ | CH₃ | 2 | 1 |
| 168 | N—COOCH₃ | C(CH₃)₂ | S | F | CH₃ | CH₃ | CH₃ | CH₃ | 2 | 1 |
| 172 | N—COOCH₃ | C(CH₃)₂ | CH₂ | F | H | H | CH₃ | CH₃ | 2 | 1 |
| 173 | N—COOCH₃ | C(CH₃)₂ | CH₂ | F | CH₃ | CH₃ | CH₃ | CH₃ | 2 | 1 |
| 174 | N—COOCH₃ | C(CH₃)₂ | CH₂ | F | H | H | H | H | 2 | 1 |
| 178 | COCH₃ | CH—CH₃ | S | F | H | H | — | — | 4 | 0 |
| 179 | N—COCF₃ | CH₂ | S | F | H | H | — | — | 4 | 0 |
| 188 | COCH₃ | CH—CF₃ | S | F | H | H | — | — | 4 | 0 |
| 191 | N—COCH₃ | CH₂ | O | F | H | H | — | — | 4 | 0 |
| 192 | N—COCH₃ | CH—CF₃ | O | F | H | H | — | — | 4 | 0 |
| 196 | N—COCH₃ | CH₂ | S | F | H | H | H | H | 4 | 1 |
| 197 | N—COCH₃ | CH₂ | S | F | H | H | — | — | 4 | 0 |
| 295 | N—COCH₃ | CH₂ | S | F | H | H | CH₃ | CH₃ | 4 | 1 |
| 296 | N—COCH₃ | CH₂ | S | F | CH₃ | CH₃ | CH₃ | CH₃ | 4 | 1 |
| 303 | N—COCH₃ | CH₂ | CH₂ | F | H | H | H | H | 4 | 1 |
| 304 | N—COOCH₃ | CH₂ | S | F | H | H | CH₃ | CH₃ | 4 | 1 |
| 305 | N—COOCH₃ | CH₂ | S | F | CH₃ | CH₃ | CH₃ | CH₃ | 4 | 1 |
| 310 | N—COOCH₃ | CH₂ | CH₂ | F | H | H | CH₃ | CH₃ | 4 | 1 |
| 311 | N—COOCH₃ | CH₂ | CH₂ | F | CH₃ | CH₃ | CH₃ | CH₃ | 4 | 1 |
| 312 | N—COOCH₃ | CH₂ | CH₂ | F | H | H | H | H | 4 | 1 |
| 313 | N—COCH₃ | CHCH₃ | S | F | H | H | CH₃ | CH₃ | 4 | 1 |
| 314 | N—COCH₃ | CHCH₃ | S | F | CH₃ | CH₃ | CH₃ | CH₃ | 4 | 1 |
| 349 | COCH₃ | C═O | S | H | H | H | — | — | 2 | 0 |
| 350 | N—COCH₃ | C—(CH₃)₂ | S | H | H | H | — | — | 2 | 0 |
| 351 | COCH₃ | CH₂ | S(O)₂ | H | H | H | — | — | 2 | 0 |
| 352 | N—COCH₃ | CH—CH₃ | S | H | H | H | — | — | 2 | 0 |
| 353 | COCF₃ | CH₂ | S | H | H | H | — | — | 2 | 0 |
| 355 | N—COCH₃ | C—(CH₃)₂ | S | H | CH₃ | CH₃ | — | — | 2 | 0 |
| 359 | COC(CH₃)₃ | CH₂ | S | H | H | H | — | — | 2 | 0 |
| 362 | N—COCH₃ | CH—CF₃ | S | H | H | H | — | — | 2 | 0 |
| 364 | COCH₃ | CF₃—C—CH₃ | O | H | H | H | — | — | 2 | 0 |
| 365 | N—COCH₃ | CH₂ | O | H | H | H | — | — | 2 | 0 |
| 366 | COCH₃ | CH—CF₃ | O | H | H | H | — | — | 2 | 0 |
| 391 | N—COCH₃ | CH(CH₃)₂ | S(O)₂ | H | H | H | — | — | 2 | 0 |
| 448 | N—COCH₃ | CH₂ | NCH₃ | H | H | H | — | — | 2 | 0 |
| 468 | N—COCH₃ | CH₂ | S | H | H | H | CH₃ | CH₃ | 2 | 1 |
| 469 | N—COCH₃ | CH₂ | S | H | CH₃ | CH₃ | CH₃ | CH₃ | 2 | 1 |
| 472 | N—COCH₃ | CH₂ | S(O)₂ | H | H | H | CH₃ | CH₃ | 2 | 1 |
| 473 | N—COCH₃ | CH₂ | S(O)₂ | H | CH₃ | CH₃ | CH₃ | CH₃ | 2 | 1 |
| 474 | N—COCH₃ | CH₂ | CH₂ | H | H | H | CH₃ | CH₃ | 2 | 1 |
| 475 | N—COCH₃ | CH₂ | CH₂ | H | CH₃ | CH₃ | CH₃ | CH₃ | 2 | 1 |
| 476 | N—COCH₃ | CH₂ | CH₂ | H | H | H | H | H | 2 | 1 |
| 477 | N—COOCH₃ | CH₂ | S | H | H | H | CH₃ | CH₃ | 2 | 1 |
| 490 | N—COCH₃ | CHCH₃ | S(O)₂ | H | H | H | CH₃ | CH₃ | 2 | 1 |
| 491 | N—COCH₃ | CHCH₃ | S(O)₂ | H | CH₃ | CH₃ | CH₃ | CH₃ | 2 | 1 |
| 492 | N—COCH₃ | CHCH₃ | CH₂ | H | H | H | CH₃ | CH₃ | 2 | 1 |
| 493 | N—COCH₃ | CHCH₃ | CH₂ | H | CH₃ | CH₃ | CH₃ | CH₃ | 2 | 1 |
| 494 | N—COCH₃ | CHCH₃ | CH₂ | H | H | H | H | H | 2 | 1 |
| 504 | N—COCH₃ | C(CH₃)₂ | S | H | H | H | CH₃ | CH₃ | 2 | 1 |
| 505 | N—COCH₃ | C(CH₃)₂ | S | H | CH₃ | CH₃ | CH₃ | CH₃ | 2 | 1 |
| 510 | N—COCH₃ | C(CH₃)₂ | CH₂ | H | H | H | CH₃ | CH₃ | 2 | 1 |
| 511 | N—COCH₃ | C(CH₃)₂ | CH₂ | H | CH₃ | CH₃ | CH₃ | CH₃ | 2 | 1 |
| 512 | N—COCH₃ | C(CH₃)₂ | CH₂ | H | H | H | H | H | 2 | 1 |
| 522 | N—COCH₃ | C═S | S | F | H | H | — | — | 2 | 0 |
| 524 | N—COCH₃ | C═S | S(O)₂ | F | H | H | — | — | 2 | 0 |
| 526 | N—COCH₃ | C═O | S(O)₂ | F | H | H | — | — | 2 | 0 |
| 530 | N—COButyl | C═O | S | F | H | H | — | — | 2 | 0 |
| 538 | N—COCH₃ | C═O | S | F | H | H | H | H | 2 | 1 |
| 562 | N—COCH₃ | C═S | O | F | H | H | — | — | 2 | 0 |
| 564 | N—COCH₂CH₃ | C═S | O | F | H | H | — | — | 2 | 0 |
| 568 | N—COCH₃ | C═S | S | F | H | H | H | H | 2 | 1 |
| 588 | N—COCH₃ | C═O | S | H | H | H | H | H | 2 | 1 |
| 589 | N—COCH₃ | C═O | S | H | H | H | CH₃ | CH₃ | 2 | 1 |
| 589 | N—COCH₃ | C═O | S | H | CH₃ | CH₃ | CH₃ | CH₃ | 2 | 1 |
| 593 | N—COCH₃ | C═O | CH₂ | H | H | H | H | H | 2 | 1 |
| 594 | N—COCH₃ | C═O | CH₂ | H | H | H | CH₃ | CH₃ | 2 | 1 |
| 598 | N—COCH₃ | C═S | CH₂ | H | H | H | CH₃ | CH₃ | 2 | 1 |
| 599 | N—COCH₃ | C═S | CH₂ | H | CH₃ | CH₃ | CH₃ | CH₃ | 2 | 1 |
| 600 | N—COCH₃ | CH- | O | F | H | H | — | — | 2 | 0 |
| | | (1R,2S,3R,4R)-- | | | | | | | | |
| | | (1,2,3,4,5)-(penta | | | | | | | | |
| | | acetoxy-pentyl | | | | | | | | |
| 615 | N—CH₃ | CH₂ | O | F | H | H | — | — | 2 | 0 |
| 616 | N—CH(CH₃)₂ | CH₂ | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 621 | N—H | CH₂ | CH₂ | F | H | H | — | — | 2 | 0 |
| 622 | N—COOC(CH₃)₃ | CH₂ | CH₂ | F | H | H | — | — | 2 | 0 |
| 623 | N—CH₃ | CH₂ | CH₂ | F | H | H | — | — | 2 | 0 |
| 624 | N—COCH₃ | CH₂ | CH₂ | F | H | H | — | — | 2 | 0 |
| 625 | N—COCF₃ | CH₂ | CH₂ | F | H | H | — | — | 2 | 0 |
| 626 | N—S(O)₂—CH₃ | CH₂ | CH₂ | F | H | H | — | — | 2 | 0 |
| 627 | N—S(O)₂—CF₃ | CH₂ | CH₂ | F | H | H | — | — | 2 | 0 |
| 628 | N—COButyl | CH₂ | CH₂ | F | H | H | — | — | 2 | 0 |
| 629 | N—CO-(2,4-diF)-Ph | CH₂ | CH₂ | F | H | H | — | — | 2 | 0 |
| 630 | N—CO-(4-OCF₃)—Ph | CH₂ | CH₂ | F | H | H | — | — | 2 | 0 |
| 631 | N—CO-(2-F, 6-CF₃)—Ph | CH₂ | CH₂ | F | H | H | — | — | 2 | 0 |
| 632 | N—COOCH₂CH(CH₃)₂ | CH₂ | CH₂ | F | H | H | — | — | 2 | 0 |
| 633 | N—COOCC(CH₃)₂ | CH₂ | CH₂ | F | H | H | — | — | 2 | 0 |
| 634 | N—COPh | CH₂ | CH₂ | F | H | H | — | — | 2 | 0 |
| 635 | N—CO(4-CF₃)—Ph | CH₂ | CH₂ | F | H | H | — | — | 2 | 0 |
| 636 | N—CO(3-CF₃)—Ph | CH₂ | CH₂ | F | H | H | — | — | 2 | 0 |
| 637 | N—CO-(2,3,6-tri-F, | CH₂ | CH₂ | F | H | H | — | — | 2 | 0 |
| | 5-5-- | | | | | | | | | |
| | OCH₃-)—Ph | | | | | | | | | |
| 638 | N—COEthyl | CH₂ | CH₂ | F | H | H | — | — | 2 | 0 |
| 639 | N—CO-n-Propyl | CH₂ | CH₂ | F | H | H | — | — | 2 | 0 |
| 640 | N—CO-i-Propyl | CH₂ | CH₂ | F | H | H | — | — | 2 | 0 |
| 641 | N—COOCH₃ | CH₂ | CH₂ | F | H | H | — | — | 2 | 0 |
| 642 | N—COO-i-Propyl | CH₂ | CH₂ | F | H | H | — | — | 2 | 0 |
| 643 | N—COO-i-Butyl | CH₂ | CH₂ | F | H | H | — | — | 2 | 0 |
| 644 | N—COOCH₃ | CH₂ | CH₂ | F | H | H | — | — | 2 | 0 |
| 645 | N—P(═O)(OEthyl)₂ | CH₂ | CH₂ | F | H | H | — | — | 2 | 0 |
| 646 | N—P(═O)(OPh)₂ | CH₂ | CH₂ | F | H | H | — | — | 2 | 0 |
| 647 | N—P(═S)(OCH₃)₂ | CH₂ | CH₂ | F | H | H | — | — | 2 | 0 |
| 648 | N-i-propyl | CH₂ | CH₂ | F | H | H | — | — | 2 | 0 |
| 649 | N-t-Butyl | CH₂ | CH₂ | F | H | H | — | — | 2 | 0 |
| 650 | 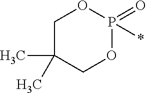 [SMILES: *P1(=O)OCC(C)(C)CO1] | CH₂ | CH₂ | F | H | H | — | — | 2 | 0 |
| 651 | 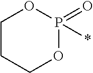 [SMILES: *P1(=O)OCCCO1] | CH₂ | CH₂ | F | H | H | — | — | 2 | 0 |
| 652 | 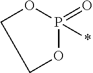 [SMILES: *P1(=O)OCCO1] | CH₂ | CH₂ | F | H | H | — | — | 2 | 0 |
| 675 | N—COCH₃ | C═O | CH₂ | F | H | H | H | H | 2 | 1 |

Compounds having general formula (I) wherein E, Z, Y, R₃, R₄, R₅, R₆, X, n and m have the meanings indicated in Table 3 are even more preferred.

TABLE 3

| Comp. | | | | | | | | | | |
|---|---|---|---|---|---|---|---|---|---|---|
| Nr. | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO(Cypropyl) | CH₂ | S | F | H | H | — | — | 2 | 0 |
| 7 | N—COCH₃ | C—(CH₃)₂ | S | F | CH₃ | CH₃ | — | — | 2 | 0 |
| 8 | N—COCH₃ | CH₃—C—COOCH₃ | S | F | H | H | — | — | 2 | 0 |
| 9 | N—COCH₃ | CH-(1R,2S,3R,4R)-- | S | F | H | H | — | — | 2 | 0 |
| | | (1,2,3,4,5)-(penta | | | | | | | | |
| | | acetoxy)-pentyl | | | | | | | | |
| 10 | N—COCH₃ | CH—(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH₃)₂ | CH₂ | S | F | H | H | — | — | 2 | 0 |
| 13 | N—CO(Cypentyl) | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 16 | N—COCH₃ | CF₃—C—CH₃ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 22 | N—COCH₃ | CH₂ | S | F | H | H | H | H | 2 | 1 |
| 23 | N—COCH₃ | CH₂ | S | F | H | H | — | — | 2 | 0 |
| 26 | N—CH₂COCH₃ | CH₂ | S | F | H | H | — | — | 2 | 0 |
| 35 | N—CH₂OCOCH₃ | CH₂ | S | F | H | H | — | — | 2 | 0 |
| 64 | N—CH₂COCH₃ | CF₃—C—CH₃ | O | F | H | H | — | — | 2 | 0 |
| 522 | N—COCH₃ | C═S | S | F | H | H | — | — | 2 | 0 |
| 562 | N—COCH₃ | C═S | O | F | H | H | — | — | 2 | 0 |
| 564 | N—COCH₂CH₃ | C═S | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 621 | N—H | CH₂ | CH₂ | F | H | H | — | — | 2 | 0 |
| 622 | N—COOC(CH₃)₃ | CH₂ | CH₂ | F | H | H | — | — | 2 | 0 |
| 623 | N—CH₃ | CH₂ | CH₂ | F | H | H | — | — | 2 | 0 |
| 624 | N—COCH₃ | CH₂ | CH₂ | F | H | H | — | — | 2 | 0 |
| 625 | N—COCF₃ | CH₂ | CH₂ | F | H | H | — | — | 2 | 0 |
| 626 | N—S(O)₂—CH₃ | CH₂ | CH₂ | F | H | H | — | — | 2 | 0 |
| 627 | N—S(O)₂—CF₃ | CH₂ | CH₂ | F | H | H | — | — | 2 | 0 |
| 629 | N—CO—(2,4-diF)—Ph | CH₂ | CH₂ | F | H | H | — | — | 2 | 0 |
| 630 | N—CO—(4-OCF₃)—Ph | CH₂ | CH₂ | F | H | H | — | — | 2 | 0 |
| 631 | N—CO—(2-F,6-CF₃)—Ph | CH₂ | CH₂ | F | H | H | — | — | 2 | 0 |
| 675 | N—COCH₃ | C═O | CH₂ | F | H | H | H | H | 2 | 1 |

wherein Cy represents the abbreviation of cycle and Ph represents the abbreviation of phenyl.

The compounds having general formula (I) can be prepared starting from the corresponding heterocyclic acid having general formula (II) by esterification reaction with a suitable alcohol having general formula (III) as indicated in reaction scheme 1, according to methods well-known in organic chemistry.

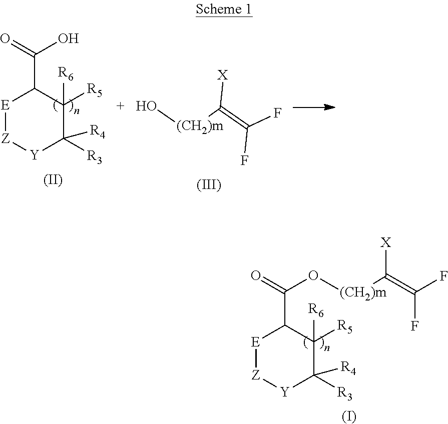

The reaction conditions provide for the use of a condenser such as, for example, N,N-dicyclohexylcarbodiimide, in the presence or absence of an amine such as N,N-dimethylaminopyridine, in an appropriate solvent such as dichloromethane, chloroform, tetrahydrofuran or dioxane, a temperature ranging from 0° C. to the boiling point of the solvent, a time ranging from 1 to 72 hours.

The compounds having formula (I) can also be obtained by reaction of the acid having formula (II) with the alcohol having formula (III) in the presence of an acid catalysis, using for example hydrochloric acid or sulfuric acid as described in R. C. Larock “Comprehensive Organic Transformations” or for example in F. T. Schevenels, M. Shen. A. Scott “J. American Chemical Society”, 2017, vol. 139 pages 6329-6337.

The compounds having formula (I) can also be obtained by Mitsunobu reaction between the acid having formula (II) and the alcohol having general formula (III) in the presence of triphenylphosphine and diethylazodicarboxylate, in a solvent such as, for example, tetrahydrofuran, diethyl ether or dioxane, at a temperature ranging from room temperature to the reflux temperature of the solvent, as described for example in U.S. Pat. No. 7,601,849 (2009).

Alternatively, the compounds having formula (I) can be obtained by activation of the carboxylic acid or via acyl chloride or via mixed anhydride and the subsequent addition of the appropriate alcohol having general formula (III), according to reaction scheme 2.

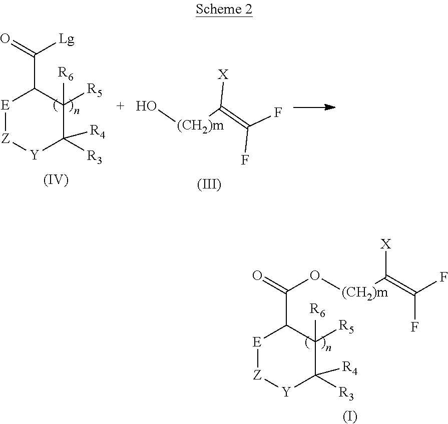

The reaction is carried out by reacting a compound having formula (IV), wherein Lg represents a chlorine atom or an OCOR_{c} residue, with R_{c} having the meaning of C₁-C₆ alkyl, obtained from the compound having general formula (II) by methods known in literature, with an alcohol having general formula (III) in the presence of a base selected from triethylamine, N-methyl-morpholine or pyridine, in a suitable solvent such as methylene chloride, chloroform or tetrahydrofuran, at a temperature ranging from 0° C. to the boiling point of the solvent, for a time ranging from 1 to 72 hours, as widely described in R C Larock “Comprehensive Organic Transformations”, in US 2003/109563 or in US 2004/198702.

The compounds having general formula (I) can also be obtained from a suitable salt of carboxylic acid, having general formula (V), a salt of an alkaline metal, such as sodium, lithium or potassium or ammonium, such as trimethylammonium or triethylammonium, in the presence of a derivative having formula (VI) wherein K represents an outgoing group such as a halogen atom, selected from chlorine, bromine or iodine or a trifluoromethanesulfonate, p-toluenesulfonate or methanesulfonate group according to reaction scheme 3.

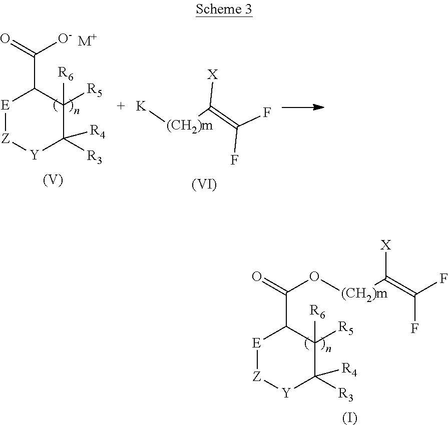

The reaction provides for the salification of carboxylic acid with a base such as sodium bicarbonate, potassium bicarbonate, potassium carbonate, sodium carbonate, sodium hydroxide, potassium hydroxide, lithium hydroxide or sodium hydride or potassium t-butylate in a solvent such as tetrahydrofuran, N,N-dimethylformamide, N-methylpyrrolidone, toluene or acetone and the subsequent addition of a compound having formula (VI), at a temperature ranging from room temperature to the reflux temperature of the solvent selected, as described for example in “Journal of Organic Chemistry” (2010) vol. 75, 4135-4145.

The compounds having formula (II), with E which represents a group having formula N-A and Z which represents the R₁—C—R₂ group or compounds having formula (VIII) wherein R₈ represents a hydrogen atom or a C₁-C₆ alkyl group, can be prepared starting from the compounds having formula (VII) by acylation or alkylation reaction with a compound A-Lg, wherein Lg represents an outgoing group such as a chlorine atom, bromine with a base such as pyridine, triethylamine, in the presence of a solvent such as tetrahydrofuran, dichloromethane, methanol, at a temperature ranging from 0° C. to the reflux temperature of the solvent selected, as described in “Journal of Organic Chemistry”, 2010, vol. 75, pages 4135-4145, and in “Bioscience, Biotechnology, and Biochemistry” (1994), vol. 58, pages 1150-1152 and indicated in reaction scheme 4.

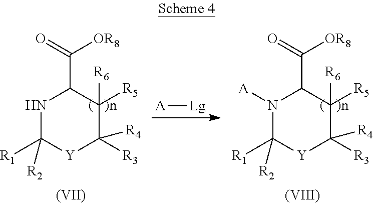

The compounds having formula (VIII) with R₈ which represents a C₁-C₆ alkyl group, can be brought back to the corresponding acid form (R₈ which represents a hydrogen atom) by acid or basic hydrolysis according to methods well-known in literature and described for example in R C Larock “Comprehensive Organic Transformations”.

The compounds having formula (VII), when they are not commercial compounds, can be prepared by the reaction of an alpha-amino acid or ester (IX), wherein R₈ represents a hydrogen atom or a C₁-C₆ alkyl group, with an aldehyde or ketone (X) in the presence of a base such as sodium bicarbonate, potassium bicarbonate, potassium carbonate, sodium carbonate, sodium hydroxide, potassium hydroxide, pyridine, in a solvent such as alcohol, water, toluene at a temperature ranging from room temperature to the reflux temperature of the solvent selected, as described in “Journal of Medicinal Chemistry”, (1976), vol. 19, pages 1002-1007 or in “Journal of Organic Chemistry” (2010), vol. 75, pages 4135-4145, or described in U.S. Pat. No. 3,980,666 (1976), WO2014120784, WO2015031627, according to reaction scheme 5.

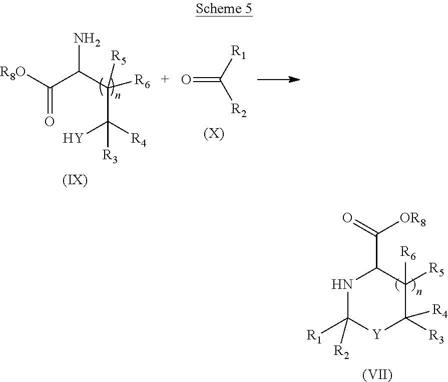

The compounds having formula (II) with Z which represents the C═O bond can be prepared by reaction of an alpha-amino acid or ester having formula (IX), wherein R₈ represents a hydrogen atom or a C₁-C₆ alkyl group, with a chloroformate having formula R_{d}OCOCl (XI), wherein R_{d} represents a phenyl or a C₁-C₆ alkyl group, in the presence of a base such as sodium bicarbonate, potassium bicarbonate, potassium carbonate, sodium carbonate, sodium hydroxide, potassium hydroxide, in a solvent such as water, toluene, xylene, tetrahydrofuran, 1,4-dioxane, diethyl ether at a temperature ranging from room temperature to the reflux temperature of the solvent selected, as described in EP1462444, US20050038260, according to reaction scheme 6.

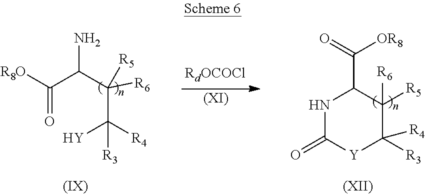

The compounds having formula (II) with Z which represents the C═S bond, when they are not commercial, can be prepared by the reaction of an alpha-amino acid or ester having formula (IX), wherein R₈ represents a hydrogen atom or a C₁-C₆ alkyl group, with carbon disulfide in the presence of a base such as sodium hydroxide, potassium hydroxide at room temperature, as described in U.S. Pat. No. 3,960,881 or with thiocarbonyldiimidazole as described in “Journal American Chemical Society” (2014), vol. 136, pages 16104-16107, according to reaction scheme 7.

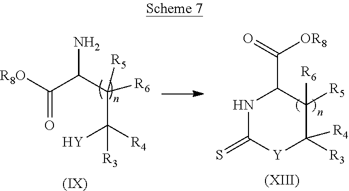

The compounds having formula (XII) and (XIII), with R₈ which represents a C₁-C₆ alkyl group, can be brought back to the corresponding acid form (R₈ which represents a hydrogen atom) by acid or basic hydrolysis according to methods well-known in literature and described for example in RC Larock “Comprehensive Organic Transformations”.

The compounds having formula (II) with E which represents a sulfur atom and Y a CH₂ group, can be obtained starting from the compounds having formula (XIV), wherein Lg represents a chlorine or bromine atom, by reaction in the presence of a strong base such as sodium or potassium methylate or potassium tert-butylate, in a suitable solvent such as dioxane or tetrahydrofuran, at a temperature ranging from room temperature to the reflux temperature of the solvent used, to give compounds having formula (XV), according to what is described for example in WO2015/005901 and indicated in scheme 8.

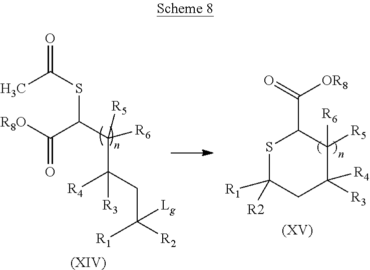

The compounds having formula (XV) with R₈ which represents a C₁-C₆ alkyl group, can be brought back to the corresponding acid form (R₈ which represents a hydrogen atom), compounds having formula (II), by acid or basic hydrolysis according to methods well-known in literature and described for example in RC Larock “Comprehensive Organic Transformations”.

The preparation of compounds having formula (XIV) is clearly described in literature, for example in WO2015/005901 or in “Chemical and Pharmaceutical Bulletin” (1986), vol. 34, pages 380-384.

The compounds having formula (II) with E which represents an oxygen atom and Y a CH₂ group, can be obtained starting from the compounds having formula (XVI), by reaction in the presence of an acid such as trifluoromethanesulfonic acid or p-toluenesulfonic, in a suitable solvent such as sulfolane, N,N-dimethylformamide or dimethylsulfoxide, at a temperature ranging from room temperature to the reflux temperature of the solvent used, to give compounds having formula (XVII) as described for example in WO2011/149339 and indicated in scheme 9.

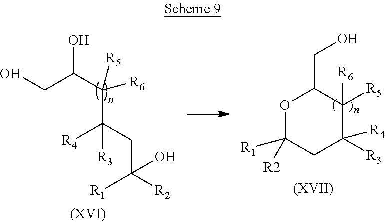

The compounds having formula (XVII) can then be oxidized to the corresponding carboxylic acids having formula (II) in the presence of an oxidizing agent, such as for example potassium permanganate, in an aqueous solution as such or in an aqueous solution in the presence of a solvent such as tetrahydrofuran o dioxane, at a temperature ranging from 0° C. to the reflux temperature, as described for example in “European Journal Organic Chemistry” (2016), vol. 2016, pages 139-149.

The compounds having formula (VI), with K which represents a halogen atom, are commercial products.

Alternatively, the compounds having formula (VI), with K which represents a trifluoromethanesulfonate, p-toluenesulfonate or methanesulfonate group, can be obtained from the corresponding alcohols (III) according to what is described in Theodora W: Greene “Protective Groups in Organic Synthesis” Third Edition pages 198-199 and indicated in scheme 10.

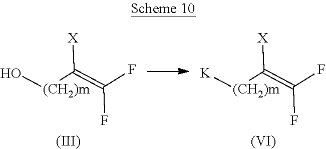

The compounds having general formula (I), for particular meanings of the substituent groups, can be obtained in racemic form or as optically active isomers.

Both compounds having general formula (I) isomerically pure, and mixtures of the same, possibly obtained during the preparation of the compounds having general formula (I) or deriving from an incomplete separation of the isomers themselves, in any proportion, therefore fall within the spirit of the present invention.

As already mentioned, the compounds having general formula (I) are provided with a high nematicidal activity and do not show any phytotoxicity with respect to the crops of application, making them suitable for use in the agrarian field in defense against nematodes.

A further object of the present invention therefore relates to the use of

compounds having formula (I) for the control of nematodes in agricultural crops.

In particular, the compounds of the present invention are effective in the control of numerous nematodes such as, for example: Pratylenchus spp, Globodera spp, Heterodera spp, Meloidogyne spp, Aphelenchoides spp, Radopholus similis Ditylenchus dipsaci, Tylenchulus semipenetrans, Longidorus spp, Xiphinema spp, Trichodorus spp, Bursaphelenchus spp, Belonolaimus spp., etc.

More specifically, the compounds having formula (I) can be applied at different times of the vegetative development, for example before the transplanting/sowing or during the growth of the plant, via the leaves, or to the soil by fertigation, or incorporation in the ground, or through seed tanning.

The compounds having formula (I) are capable of exerting a curative and preventive nematocidal action and exhibit a very low or no phytotoxicity on the crops treated.

For practical uses in agriculture, it is often advantageous to use the compounds of the present invention appropriately formulated in agronomic compositions having a nematicidal activity comprising one or more compounds having formula (I), possibly also as a mixture of agronomically acceptable isomers and coformulants.

A further object of the present invention therefore relates to nematocidal agronomic compositions comprising one or more compounds having formula (I), a solvent and/or solid, liquid or liquefied diluent, optionally one or more surfactants and other agronomically acceptable coformulants.

Compositions can be used which are in the form of dry powders, wettable powders, emulsifiable concentrates, microemulsions, pastes, granulates, solutions, suspensions, fumigants etc.: the choice of the type of composition will depend on the specific use.

The compositions are prepared according to known methods, for example by diluting or dissolving the active substance with a solvent and/or solid diluent, optionally in the presence of surfactants.

Kaolin, alumina, silica, talc, bentonite, gypsum, quartz, dolomite, attapulgite, montmorillonite, diatomaceous earth, cellulose, starch, etc. can be used as solid inert diluents, or carriers.

Liquid inert diluents that can be used are water, or organic solvents such as aromatic hydrocarbons (xylols, mixtures of alkylbenzenes, etc.), aliphatic hydrocarbons (hexane, cyclohexane, etc.), halogenated aromatic hydrocarbons (chlorobenzene, etc.), alcohols (methanol , propanol, butanol, octanol, etc.), esters (isobutyl acetate, etc.), ketones (acetone, cyclohexanone, acetophenone, isophorone, ethylamylketone, etc.), or vegetable or mineral oils or mixtures thereof, etc.

Propellant gases such as butane, propane, halogenated hydrocarbons, nitrogen or carbon dioxide can be used as liquefied diluents or liquefied substances that gasify at room temperature and pressure.

Surfactants that can be used are wetting agents and emulsifiers of the non-ionic type (polyethoxylated alkylphenols, polyethoxylated fatty alcohols, etc.), anionic type (alkylbenzene sulfonates, alkylsulfonates, etc.), cationic type (quaternary salts of alkylammonium, etc.).

Dispersants (e.g. lignin and its salts, cellulose derivatives, alginates, etc.), stabilizers (e.g. antioxidants, ultraviolet ray absorbents, etc.) can also be added.

The concentration of active substance in the above compositions can vary within a wide range, depending on the active compound, the applications for which they are intended, the environmental conditions and the type of formulation adopted. In general, the concentration of active substance preferably ranges from 0.1 to 90%, and in particular from 0.5 to 90%.

The compounds of the present invention, as such or formulated, can be used in a mixture with other active ingredients such as, for example, herbicides, fungicides, bactericides, insecticides, acaricides, nematocides, fertilizers, biostimulants, etc. to broaden the spectrum or prevent resistance.

In some cases, the mixtures thus obtained have a synergistic effect between the components, which brings the mixture, for example, to exert a higher activity with respect to that of the individual elements of which it is composed.

Examples of insecticides, acaricides, nematocides that can be added to the compositions containing one or more compounds having general formula (I) are the following: abamectin, acetamiprid, acrinathrin, alphacypermethrin, alphamethrin, azadirachtin, Bacillus subtilis, Bacillus thuringiensis, Beauveria bassiana, betacyfluthrin, bifenazate, bifenthrin, buprofezin, chlorpyrifos, chlorpyrifos M, clofentezine, cyhalothrin, cyhexatin, cypermethrin, cyromazine, chloropicrin, clorantranilipide, clotianidin, deltamethrin, diflubenzuron, dimethoat, dazonet, difluoruro di solforile, dimethyldisulfide, emamectin, esfenvalerate, ethoprophos, etofenprox, etoxazole, fenamiphos, fenazaquin, fenoxycarb, fenpyroximate, fipronil, fluazaindolizine, fluazinam, fluensulfone, flufenoxuron, fluvalinate, fluopyram, fosthiazate, formentanate, flonicamid, formet, viruses, hexythiazox, imidaclopridi, indoxacarb, lambda-cyhalothrin, lufenuron malathion, metaldehyde, methamidophos, Metharhizium spp, methiocarb, methomyl, methoxyfenozide, milbemectin, metaflumizone, metam sodium, metam potassium, oxamyl, Paecilomyces fumosoroseus, phosmet, pirimicarb, pirimiphos M, pyrethrum, pyridaben, pyriproxyfen, piperonyl butoxide, spinosad, spironesifen, spirotetramat, spinetoran, spirodiclofen, tau-fluvalinate, tebufenozide, tebufenpyrad, teflubenzuron, tefluthrin, thiacloprid, triflumuron, zeta-cypermethrin, (1R-cis)-[5-(phenylmethyl)-3-furanyl]-methyl-3-[(dihydro-2-oxo-3(2H)-furanylidene) methyl]-2,2-dimethylcyclopropanecarboxylate, (3-phenoxyphenyl)-methyl-2,2,3,3-tetramethyl-cyclopropanecarboxylate, 1-[(2-chloro-5-thiazolyl)methyl]-5-triazine-2-(1H)-imine, 2-(2-chloro-6-fluorophenyl)-4-[4-(1,1-dimethylethyl)phenyl]-4,5-dihydro-oxazole, 2-(acetyloxy)-3-dodecyl-1,4-naphthalenedione, 2-chloro-N-[[[4-(1-phenylethoxy)-phenyl]-amino]-carbonyl]-benzamide, 2-chloro-N-[[[4-(2,2-dichloro-1,1-difluoroethoxy)-phenyl]-amino]-carbonyl]-benzamide, 3-methylphenyl-propylcarbamate, 4-[4-(4-ethoxyphenyl)-4-methylpentyl]-1-fluoro-2-phenoxybenzene, 4-chloro-2-(1,1-dimethylethyl)-5-[[2-(2,6-dimethyl-4-phenoxyphenoxy)ethyl]thio]-3-(2H)-pyridazinone, 4-chloro-2-(2-chloro-2-methylpropyl)-5-[(6-iodo-3-pyridinyl)(2-chloro-2-methylpropyl)-5-[(6-iodo-3-pyridinyl)-methoxy]-3-(2H)pyridazinone, 4-chloro-5-[(6-chloro-3-pyridinyl)mehoxy]-2-(3,4-dichlorophenyl)-3(2H)pyridazinone, Bacillus thuringiensis strain EG-2348, [2-benzoyl-1-(1,1-dimethylethyl)-hydrazine] benzoic acid, 2,2-dimethyl-3-(2,4-dichlorophenyl)-2-oxo-1-oxaspiro [4.5]-dec-3-en-4-yl butanoate, [3-[(6-chloro-3-pyridinyl)-methyl]-2-thiazolidinylidene]-cyanamide, dihydro-2-(nitromethylene)-2H-1,3-thiazine-3(4H)-carboxaldehyde, ethyl[2-[[1,6-dihydro-6-oxo-1-(phenylmethyl)-4-pyridazinyl]oxy]ethyl]-carbammate, N-(3,4,4-trifluoro-1-oxo-3-butenyl)-glycine, N-(4-chlorophenyl)-3-[4-(difluoromethoxy)-phenyl]-4,5-dihydro-4-phenyl-1H-pyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl[2-(dipropylamino)-2-oxoethyl]-ethyl-phosphoroamidothioate.

Examples of herbicides that can be added to the compositions containing one or more compounds having general formula (I) are the following: acetochlor, acifluorfen, aclonifen, AKH-7088 ({metil (E,Z)-[[[1-[2-chloro-4-(trifluoromethyl)phenoxy]-2-nitrophenyl]-2-methoxyethylidene]amino]acetate}), alachlor, alloxydim, ametryn, amicarbazone, amidosulfuron, amitrole, anilofos, asulam, atrazine, azafenidin, azimsulfuron, aziprotryne, BAY MKH 6561 (methyl 2-({[(4-methyl-5-oxo-3-propoxy-4,5-dihydro-1H-1,2,4-triazol-1-yl)carbonyl] amino}sulfonyl)benzoate sodium salt), beflubutamid, benazolin, benfluralin, benfuresate, bensulfuron, bensulide, bentazone, benzfendizone, benzobicyclon, benzofenap, benzthiazuron, bifenox, bilanafos, bispyribac-sodium, bromacil, bromobutide, bromofenoxim, bromoxynil, butachlor, butafenacil, butamifos, butenachlor, butralin, butroxydim, butylate, cafenstrole, carbetamide, carfentrazone-ethyl, chlomethoxyfen, chloramben, chlorbromuron, chlorbufam, chlorflurenol, chloridazon, chlorimuron, chlornitrofen, chlorotoluron, chloroxuron, chlorpropham, chlorsulfuron, chlorthal, chlorthiamid, cinidon ethyl, cinmethylin, cinosulfuron, clethodim, clodinafop, clomazone, clomeprop, clopyralid, cloransulam-methyl, cumyluron (JC-940), cyanazine, cycloate, cyclosulfamuron, cycloxydim, cyhalofop-butyl, 2,4-D, 2,4-DB, daimuron, dalapon, desmedipham, desmetryn, dicamba, dichlobenil, dichlorprop, dichlorprop-P, diclofop, diclosulam, diethatyl, difenoxuron, difenzoquat, diflufenican, diflufenzopyr, dimefuron, dimepiperate, dimethachlor, dimethametryn, dimethenamid, dinitramine, dinoseb, dinoseb acetate, dinoterb, diphenamid, dipropetryn, diquat, dithiopyr, 1-diuron, eglinazine, endothal, EPTC, esprocarb, ethalfluralin, ethametsulfuron-methyl, ethidimuron, ethiozin (SMY 1500), ethofumesate, ethoxyfen-ethyl (HC-252), ethoxysulfuron, etobenzanid (HW 52), fenoxaprop, fenoxaprop-P, fentrazamide, fenuron, flamprop, flamprop-M, flazasulfuron, florasulam, fluazifop, fluazifop-P, fluazolate (JV 485), flucarbazone-sodium, fluchloralin, flufenacet, flufenpyr ethyl, flumetsulam, flumiclorac-pentyl, flumioxazin, flumipropin, fluometuron, fluoroglycofen, fluoronitrofen, flupoxam, flupropanate, flupyrsulfuron, flurenol, fluridone, flurochloridone, fluroxypyr, flurtamone, fluthiacet-methyl, fomesafen, foramsulfuron, fosamine, furyloxyfen, glufosinate, glyphosate, halosulfuron-methyl, haloxyfop, haloxyfop-P-methyl, hexazinone, imazamethabenz, imazamox, imazapic, imazapyr, imazaquin, imazethapyr, imazosulfuron, indanofan, iodosulfuron, ioxynil, isopropalin, isoproturon, isouron, isoxaben, isoxachlortole, isoxaflutole, isoxapyrifop, KPP-421, lactofen, lenacil, linuron, LS 830556 ([[[2-methyl(methylsulfonyl)amino]-2-oxoethyl]amino]-methylphosphonic acid), CPA (2-methyl-4-chlorophenoxyacetic acid), MCPA-thioethyl, MCPB (4-(4-chloro-2-methylphenoxy)butanoic acid), mecoprop, mecoprop-P, mefenacet, mesosulfuron, mesotrione, metamitron, metazachlor, methabenzthiazuron, methazole, methoprotryne, methyldymron, metobenzuron, metobromuron, metolachlor, S-metolachlor, metosulam, metoxuron, metribuzin, metsulfuron, molinate, monalide, monolinuron, naproanilide, napropamide, naptalam, NC-330 (methyl 5-[(4,6-dimethylpyrimidin-2-yl)carbamoylsulfamoyl]1-pyridin-2-yl-pyrazole-4-carboxylate), neburon, nicosulfuron, nipyraclofen, norflurazon, orbencarb, oryzalin, oxadiargyl, oxadiazon, oxasulfuron, oxaziclomefone, oxyfluorfen, paraquat, pebulate, pendimethalin, penoxsulam, pentanochlor, pentoxazone, pethoxamid, phenmedipham, picloram, picolinafen, piperophos, pretilachlor, primisulfuron, prodiamine, profluazol, proglinazine, prometon, prometryne, propachlor, propanil, propaquizafop, propazine, propham, propisochlor, propyzamide, prosulfocarb, prosulfuron, pyraclonil, pyraflufen-ethyl, pyrazogyl (HSA-961), pyrazolynate, pyrazosulfuron, pyrazoxyfen, pyribenzoxim, pyributicarb, pyridafol, pyridate, pyriftalid, pyriminobac-methyl, pyrithiobac-sodium, pyroxasulfone quinclorac, quinmerac, quizalofop, quizalofop-P, rimsulfuron, sethoxydim, siduron, simazine, simetryn, sulcotrione, sulfentrazone, sulfometuron-methyl, sulfosulfuron, 2,3,6-TBA, TCA-sodium, tebutam, tebuthiuron, tepraloxydim, terbacil, terbumeton, terbuthyl-azine, terbutryn, thenylchlor, thiazafluron, thiazopyr, thidiazimin, thifensulfuron-methyl, thiobencarb, tiocarbazil, tioclorim, tralkoxydim, tri-allate, triasulfuron, triaziflam, tribenuron, triclopyr, trietazine, trifloxysulfuron, trifluralin, triflusulfuron-methyl, tritosulfuron, UBI-C4874 (quizalofop-P), vernolate.

Examples of fungicides that can be added to the compositions containing one or more compounds having general formula (I) are the following: acibenzolar, ametoctradin, amisulbrom, ampropylfos, anilazine, azaconazole, azoxystrobin, benalaxyl, benalaxyl-M, benomyl, benthiavalicarb, bitertanol, bixafen, blasticidin-S, boscalid, bromuconazole, bupirimate, buthiobate, captafol, captan, carbendazim, carboxin, carpropamid, chinomethionat, chloroneb, chlorothalonil, chlozolinate, cyazofamid, cyflufenamid, cymoxanil, cyproconazole, cyprodinil, debacarb, dichlofluanid, dichlone, diclobutrazol, diclomezine, dicloran, diclocymet, diethofencarb, difenoconazole, diflumetorim, dimethirimol, dimethomorph, dimoxystrobin, diniconazole, dinocap, dipyrithione, ditalimfos, dithianon, dodemorph, dodine, edifenphos, epoxiconazole, etaconazole, ethaboxam, ethirimol, ethoxyquin, etridiazole, famoxadone, fenamidone, fenaminosulf, fenapanil, fenarimol, fenbuconazole, fenfuram, fenhexamid, fenoxanil, fenpiclonil, fenpicoxamid, fenpropidin, fenpropimorph, fenpyrazamine, fentin, ferbam, ferimzone, florylpicoxamid, fluazinam, fludioxonil, fluindapyr, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpet, fosetyl-aluminium, fuberidazole, furalaxyl, furametpyr, furconazole, furconazole-cis, guazatine, hexaconazole, hymexazol, hydroxyquinoline sulfate, imazalil, imibenconazole, iminoctadine, inpyrfluxam, ipconazole, ipfentrifluconazole, iprobenfos, iprodione, isoprothiolane, iprovalicarb, isopyrazam, isotianil, kasugamycin, kresoxim-methyl, mancopper, mancozeb, mandipropamid, maneb, mebenil, mefentrifluconazole, mepanipyrim, mepronil, meptyldinocap, metalaxyl, metalaxyl-M, metconazole, methfuroxam, metiram, metominostrobin, metrafenone, metsulfovax, myclobutanil, natamycin, nicobifen, nitrothal-isopropyl, nuarimol, ofurace, orysastrobin, oxadixyl, oxpoconazole, oxycarboxin, pefurazoate, penconazole, pencycuron, penflufen, pentachlorofenol e suoi sali, penthiopyrad, phthalide, picoxystrobin, piperalin, Bordeaux mixture, polyoxins, probenazole, prochloraz, procymidone, propamocarb, propiconazole, propineb, proquinazid, prothiocarb, prothioconazole, pyracarbolid, pyraclostrobin, pyrametostrobin, pyraoxystrobin, pyrazophos, pyribencarb, pyrifenox, pyrimethanil, pyriofenone, pyroquilon, pyroxyfur, quinacetol, quinazamid, quinconazole, quinoxyfen, quintozene, rabenzazole, copper hydroxide, copper oxychloride, copper (I) oxide, copper sulfate, sedaxane, silthiofam, simeconazole, spiroxamine, streptomycin, tebuconazole, tebufloquin, tetraconazole, thiabendazole, thiadifluor, thicyofen, thifluzamide, thiophanate, thiophanate-methyl, thiram, tiadinil, tioxymid, tolclofos-methyl, tolylfluanid, triadimefon, triadimenol, triarimol, triazbutil, triazoxide, tricyclazole, tridemorf, trifloxystrobin, triflumizole, triforine, triticonazole, uniconazole, uniconazole-P, validamycin, valifenalate, vinclozolin, zineb, ziram, zolfo, zoxamide.

Examples of bactericides that can be added to the compositions containing one or more compounds having general formula (I) are the following: bronopol, dichlorophen, nitrapyrina, nickel dimethyldithiocarbamate, kasugamycin, octhilinone, furancarboxylic acid, probenazole, streptomycin, teclophthalam, copper hydroxide, copper oxychloride, copper (I) oxide, copper sulfate, copper salicylate.

Examples of fertilizers and biostimulants that can be added to the compositions containing one or more compounds having general formula (I) are the following: mixtures of amino acids and/or oligopeptides of an animal and/or vegetable origin, 4-thiazolidinecarboxylic acid, 4-acetylthiazolidine-carboxylic acid, ectoin, phytosterols.

The present invention therefore also relates to agronomic compositions comprising at least one compound having formula (I) and at least one second active ingredient selected from insecticides, acaricides, nematocides other than those having formula (I), herbicides, fungicides, bactericides, fertilizers and biostimulants.

The compositions, object of the present invention, are capable of exerting a nematicidal action which can be of a curative and/or preventive nature and, in general, exhibit very low or no phytotoxicity with respect to the crops treated.

The present invention therefore further relates to the use of compositions comprising at least one compound having formula (I) for the control of nematodes in agricultural crops.

If the compositions comprise a compound having formula (I) and at least one other known active ingredient, the weight ratios in the above compositions, between the compound having formula (I) and the other known active ingredients, vary according to the compounds selected and can normally be within the range of 1:100 to 100:1, preferably from 1:10 to 10:1.

The total concentration of the active components in the above compositions can vary within a wide range; it generally ranges from 1% to 99% by weight with respect to the total weight of the composition, preferably from 5% to 90% by weight with respect to the total weight of the composition.

The compounds having formula (I) or the compositions containing them can be applied to the crop via the leaves, or to the soil by fertigation, or incorporation in the ground, or through seed tanning.

The present invention therefore also relates to a method for controlling nematodes in cultivated areas, which consists in applying effective and non-phytotoxic doses of compositions comprising at least one compound having formula (I) and, optionally, one or more known active ingredients compatible therewith, on any part of the plant to be protected.

The quantity of compound to be applied for obtaining the desired effect can vary depending on various factors such as, for example, the compound used, the crop to be protected, the degree of infestation, the climatic conditions, the characteristics of the soil, the method of application, etc.

Doses of compound having formula (I) ranging from 100 g to 10,000 g per hectare of agricultural crop or, in the case of compositions comprising other known active ingredients, overall doses of active ingredients ranging from 100 g to 20,000 g per hectare of agricultural crop, generally provide a sufficient control.

Doses of the compound having formula (I) ranging from 500 g to 800 g per hectare of agricultural crop are preferably used.

The following examples are now provided for a better illustration of the present invention, which should be considered illustrative and non-limiting thereof.

### EXAMPLE 1

### Preparation of (2,2,5,5-tetramethyl)-1,3-thiadiazolidine-4-carboxylic acid (Intermediate Having formula (VII))

2.0 g (13.81 mmoles) of (D,L)-penicillamine and 13.81 ml of acetone were introduced into a reaction flask in a nitrogen atmosphere. The reaction mixture was heated under reflux for 1 hour. After LC-MS control, the solvent was evaporated under reduced pressure. 2.6 g of a white solid were obtained which was used as such in the subsequent reaction.

### EXAMPLE 2

### Preparation of (N-acetyl-2,2,5,5-tetramethyl)-1,3-thiadiazolidine-4-carboxylic acid (Intermediate Having formula (II))

137 ml of tetrahydrofuran and 5.7 ml (41.25 mmoles) of triethylamine were added to the compound obtained in the previous reaction.

The reaction mixture was brought to 0° C. with an ice-salt bath and 1.95 ml of acetyl chloride were added dropwise.

At the end of the dripping, the mixture was left under magnetic stirring at room temperature for 24 hours.

After LC-MS control, the mixture was diluted with water, then acidified with 10% diluted HCl and the phases were separated; the aqueous phase was re-extracted twice with ethyl acetate. After drying on sodium sulfate, filtration and evaporation of the solvent under reduced pressure, 3.0 g of solid were obtained, which were used without further purification in the subsequent reaction.

### EXAMPLE 3

### Preparation of (N-acetyl 2,2,5,5-tetramethyl)-1,3-thiadiazolidine-4-carboxylate of 3,4,4-trifluoro-3-butenyl (Compound Nr. 7)

3.0 g (12.98 mmoles) of (N-acetyl 2,2,5,5-tetramethyl)-1,3-thiadiazolidine-4-carboxylic acid were dissolved, in a nitrogen atmosphere, in 21 ml of N,N-dimethylformamide. 2.68 g (19.47 mmoles) of potassium carbonate and 2.24 ml (19.47 mmoles) of 1-bromine-3,4.4-trifluoro-3-butene were added under stirring and keeping the reaction mixture at room temperature. The reaction mixture was then left under magnetic stirring at room temperature for 24 hours.

After LC-MS control, the mixture was diluted with water and the phases were separated; the aqueous phase was re-extracted twice with ethyl acetate. The combined organic phases were washed abundantly with a saturated solution of sodium chloride and subsequently with water. After anhydrification on sodium sulfate, filtration and evaporation of the solvent under reduced pressure, 4.5 g of orange oil were obtained. The raw product thus obtained was purified by chromatography on silica gel eluting with a 5:5 heptane:ethyl acetate mixture, obtaining 2.5 g of the desired product. Yield 56%
LC-MS [M+H]=339

### EXAMPLE 4

### Preparation of [2-(1R,2S,3R,4R)-(1,2,3,4,5-pentahydroxypentyl)]-1,3-thiazolidine-4-carboxylic acid (Intermediate Having Formula (VII))

2.57 ml (31.7 mmoles) of pyridine were added to a solution of 5.0 g (31.7 mmoles) of (L)-cysteine hydrochloride and 5.71 g (31.7 mmoles) of (D)-glucose in 10 ml of water.

The reaction was left under magnetic stirring at room temperature for 24 hours, 50 ml of ethanol were then added and the mixture was left under magnetic stirring for one hour. The solid precipitate was filtered and dried under reduced pressure to obtain 5.2 g of white solid, used as such in the subsequent reaction.

### EXAMPLE 5

### Preparation of [N-acetyl-2-(1R,2S,3R,4R)-(1,2,3,4,5-pentacetoxypentyl)]-1,3-thiazolidine-4-carboxylic acid (Intermediate Having Formula (II))

32 ml of acetic anhydride were added dropwise in a nitrogen atmosphere to 45 ml of pyridine at 0° C. keeping the temperature below 5° C. [2-(1R,2S,3R,4R)-(1,2,3,4,5-pentahydroxypentyl)]-1,3-thiazolidine-4-carboxylic acid (5.2 g) obtained in Example 4 was added to the solution. The reaction mixture was left under magnetic stirring for 24 hours.

After LC-MS control, the solvent was evaporated under reduced pressure.

An extremely thick oil was obtained, which was taken up with heptane and evaporated under reduced pressure to obtain 4.5 g of white solid. Yield 75%.

### EXAMPLE 6

### [N-acetyl-2-(1R,2S,3R,4R)-(1,2,3,4,5-pentacetoxypentyl)]-1,3,4-thiazolidin-4-carboxylate of 3,4,4-trifluoro-3-butenyl (Compound Nr. 9)

2.0 g (3.74 mmoles) of [N-acetyl-2-(1R,2S,3R,4R)-(1,2,3,4,5-pentacetoxypentyl)]-1,3-thiazolidine-4-carboxylic acid were dissolved, in a nitrogen atmosphere, in 6.23 ml of N,N-dimethylformamide. 0.77 g (5.61 mmoles) of potassium carbonate and 0.64 ml (5.61 mmoles) of 1-bromine-3,4,4-trifluoro-3-butene were added under stirring and keeping the reaction mixture at room temperature. The mixture was left under magnetic stirring at room temperature for 24 hours.

After LC-MS control, the mixture was diluted with water and the phases were then separated; the aqueous phase was re-extracted twice with ethyl acetate. The combined organic phases were washed abundantly with a saturated sodium chloride solution and subsequently with water. After drying on sodium sulfate, filtration and evaporation of the solvent under reduced pressure, 2.0 g of an orange oil were obtained which was purified by silica gel chromatography eluting with a heptane:ethyl acetate 3:7 mixture, obtaining 1.2 g of the desired product. Yield 50%
LC-MS [M+H]=643

### EXAMPLE 7

### Preparation of (2-trifluoromethyl-2-methyl)-1,3-oxazolidine-4-methyl carboxylate (Intermediate Having Formula (VII))

1.0 g (12.28 mmoles) sodium acetate and 3,45 ml (38.56 mmoles) of trifluoroacetone were added to a solution of 13.00 g (12.28 mmoles) of the methyl ester of (S)-serine hydrochloride in 4.50 ml of toluene. The reaction mixture was left under magnetic stirring at room temperature for 6 hours; 25.78 ml of toluene were then added and the mixture was heated under reflux for 18 hours using a Dean-Stark apparatus.

After GC-MS control, the reaction mixture was evaporated under reduced pressure, obtaining a raw product that was used as such in the subsequent reaction.

### EXAMPLE 8

### Preparation of (N-acetyl-2-trifluoromethyl-2-methyl)-1,3-oxazolidine-4-methyl carboxylate (Intermediate Having Formula (VIII))

Catalytic iodine was added to a solution of (2-trifluoromethyl-2-methyl)-1,3-oxazolidine-4-methyl carboxylate 2.5 g (11.73 mmoles) in 11.09 ml of acetic anhydride, and the mixture was left under magnetic stirring at room temperature for 24 hours. After LC-MS control, the reaction mixture was evaporated under reduced pressure. The raw product obtained was used in the subsequent reaction.

### EXAMPLE 9

### Preparation of the Lithium Salt of (N-acetyl-2-trifluoromethyl-2-methyl)-1,3-oxazolidine-4-carboxylic acid (Intermediate Having Formula (V))

12.50 ml of an aqueous solution containing 0.30 g (12.50 mmoles) of lithium hydroxide were added, under stirring at 0° C. to a solution of 2.9 g (11.37 mmoles) of (N-acetyl-2-trifluoromethyl-2-methyl)-1,3-oxazolidine-4-methyl carboxylate in 18 ml of tetrahydrofuran. The reaction mixture was then brought to room temperature and left under stirring overnight.

The following day, the solvent was evaporated and the lithium salt of N-acetyl-2-trifluoromethyl-2-methyl)-1,3-oxazolidine-4-carboxylic acid was used as such for the subsequent reaction.

### EXAMPLE 10

### Preparation of (N-acetyl-2-trifluoromethyl-2-methyl)-1,3-oxazolidine-4-carboxylate of 3,4,4-trifluoro-3-butenyl (Compound Nr. 16)

2.9 g (9.04 mmoles) of the lithium salt of (N-acetyl-2-trifluoromethyl-2-methyl)-1,3-oxazolidine-4-carboxylic acid, obtained in the previous reaction, were suspended in 15 ml of N,N-dimethylformamide, subsequently adding 1.87 g (13.56 mmoles) of potassium carbonate and 1.56 ml (13.56 mmoles) of 1-bromo-3,4,4-trifluoro-3-butene. The reaction mixture was left under magnetic stirring at room temperature overnight.

After LC-MS control, the reaction mixture was diluted with water; the phases were then separated and the aqueous phase was re-extracted twice with ethyl acetate. The combined organic phases were washed abundantly with a saturated sodium chloride solution and subsequently with water. After anhydrification on sodium sulfate, filtration and evaporation of the solvent under reduced pressure, 2.1 g of an orange oil were obtained, then purified by chromatography on silica gel eluting with a heptane:ethyl acetate 6:4 mixture, obtaining 1.1 g of the desired product.
LC-MS [M+H]=349

### EXAMPLE 11

### Preparation of N-acetyl-1,3-thiadiazolidine-4-carboxylate of 3,4,4-trifluoro-3-butenyl (Compound Nr. 23)

10.0 g (57.14 mmoles) of N-acetyl-1,3-thiadiazolidine-4-carboxylic acid in 95 ml N,N-dimethylformamide were introduced, in a nitrogen atmosphere, into a reaction flask; 11.83 g (85.71 mmoles) of potassium carbonate and 9.87 ml (85.71 mmoles) of 1-bromo-3,4,4-trifluoro-3-butene were then added. The reaction mixture was left under magnetic stirring at room temperature overnight. After LC-MS control, the reaction mixture was diluted with water, the phases were separated and the aqueous phase was re-extracted twice with ethyl acetate. The combined organic phases were washed abundantly with a saturated sodium chloride solution and subsequently with water. After anhydrification on sodium sulfate, filtration and evaporation of the solvent under reduced pressure, 19 g of an orange oil were obtained, purified by chromatography on silica gel eluting with a heptane:ethyl acetate 3:7 mixture, obtaining 15 g of the desired product.
LC-MS [M+H]=283

### EXAMPLE 12

### Preparation of N-(tert-butoxycarbonyl)-L-proline 3,4,4-trifluorobut-3-en-1-yl ester (Compound Nr. 622)

A solution of 3,4,4-trifluorobut-3-en-1-ol (2.79 g, 22.12 mmoles) was prepared in an anhydrous environment and under a nitrogen atmosphere, in 109 ml of anhydrous CH₂C₁₂; the solution was subsequently brought to 0° C., 5.0 g (23.23 mmoles) of N-(tert-butoxycarbonyl)-L-proline, 270 mg (2.21 mmoles) of DMAP and 7.0 g (36.50 mmoles) of EDCI were then added in sequence.

The mixture was stirred and left to return to room temperature. After 48 hours, the reaction progress was controlled by LC-MS analysis, with confirmation of the formation of the product. The reaction was then recovered by diluting with dichloromethane and washing with a saturated solution of sodium bicarbonate (twice), subsequently with a solution of 1 N of HCl cooled to 0° C., and then with brine. The organic phase was dried with sodium sulfate and after filtration and evaporation, 7.67 g of the desired product were obtained (quantitative yield).
LC-MS [M+H⁺]=324.1

### EXAMPLE 13

### Preparation of L-proline 3,4,4-trifluorobut-3-en-1-yl ester hydrochloride (Compound Nr. 621)

A solution of 5.17 g (16.01 mmoles) of N-(tert-butoxycarbonyl)-L-proline 3,4,4-trifluorobut-3-en-1-yl ester in 79 ml of anhydrous dichloromethane was prepared in an anhydrous environment and under a nitrogen atmosphere. 40 ml of a 4 N HCl solution in dioxane (160 mmoles) were then added, and the mixture was left under stirring for 2 hours. After LC-MS control, with confirmation of the transformation, the reaction solvent was evaporated and the residue was brought to dryness. 4 g of the desired product were obtained as a yellow oil (quantitative yield).
LC-MS [M+H⁺]=224.1.

### EXAMPLE 14

### Preparation of N-methyl-L-proline 3,4,4-trifluorobut-3-en-1-yl ester (Compound Nr. 623)

760 mg (2.95 mmoles) of L-proline 3,4,4-trifluorobut-3-en-1-yl ester hydrochloride were dissolved in an anhydrous environment in 9.8 ml of anhydrous acetone, 840 mg of methyl iodide (0.37 ml, 5.89 mmoles) and 0.82 ml (5.89 mmoles) of triethylamine were subsequently added at room temperature. The reaction mixture was left under stirring for 48 hours; the reaction solvent was subsequently evaporated and the residue was diluted with water and washed with dichloromethane (twice). The aqueous phase containing the product of interest was basified up to pH 8-9 with NaHCO₃ (saturated solution), then extracted with dichloromethane (3 times). After anhydrification, filtration and evaporation, 350 mg of the desired product were obtained as a yellow oil (yield=50%).
LC-MS [M+H⁺]=238.1.

### EXAMPLE 15

### Preparation of N-acetyl-L-proline 3,4,4-trifluorobut-3-en-1-yl ester (Compound Nr. 624)

The substrate (L-proline 3,4,4-trifluorobut-3-en-1-yl ester hydrochloride) (770 mg, 2.98 mmoles) was dissolved in 6 ml of pyridine under a nitrogen atmosphere; 1.41 ml (14.9 mmoles) of acetic anhydride were subsequently added. The reaction was stirred for 48 hours. The reaction was subsequently recovered by evaporating the reaction solvent; the residue thus obtained was taken up with water and extracted twice with dichloromethane. The combined organic phases were washed with brine. After anhydrification, filtration and evaporation, 750 mg of the desired product were obtained as a yellow oil (yield=95%).
LC-MS [M+H⁺]=266.1.

### EXAMPLE 16

### Preparation of N-trifluoroacetyl-L-proline 3,4,4-triflluorobut-3-en-1-yl ester (Compound Nr. 625)

The substrate (L-proline 3,4,4-trifluorobut-3-en-1-yl ester hydrochloride) (690 mg, 2.66 mmoles) was dissolved in 5.3 ml of pyridine under a nitrogen atmosphere; 1.85 ml (13.29 mmoles) of trifluoroacetic anhydride were subsequently added at 0° C. The reaction was stirred for 16 hours. The reaction was subsequently recovered by evaporating the reaction solvent; the residue thus obtained was taken up with water and extracted twice with dichloromethane. The combined organic phases were washed with brine. After anhydrification, filtration and evaporation, 900 mg of the desired product were obtained as a yellow oil (quantitative yield).
LC-MS [M+H⁺]=320.1.

### EXAMPLE 17

### Preparation of N-methylsulfonyl-L-proline 3,4,4-triflluorobut-3-en-1-yl ester (Compound Nr. 626)

The substrate (L-proline 3,4,4-trifluorobut-3-en-1-yl ester hydrochloride) (770 mg, 2.97 mmoles) was dissolved in 9.9 ml of dichloromethane under a nitrogen atmosphere, 0.91 ml (6.52 mmoles) of triethylamine were added and the reaction solution was then cooled to 0° C. 0.28 ml (3.56 mmoles) of methanesulfonyl chloride were subsequently added and the mixture was left under stirring for 16 hours. After LC-MS control, the reaction proved to be complete; the reaction was recovered by adding water to the reaction mixture and then extracting with dichloromethane (3 times). The combined aqueous phases were washed with brine. After anhydrification with sodium sulfate, filtration and evaporation, 850 mg of the desired product were obtained as a yellow oil (yield=95%).
LC-MS [M+H⁺]=302.1.

### EXAMPLE 18

### Preparation of N-trifluoromethylsulfonyl-L-proline 3,4,4-triflluorobut-3-en-1-yl ester (Compound Nr. 627)

The substrate (L-proline 3,4,4-trifluorobut-3-en-1-yl ester hydrochloride) (530 mg, 2.04 mmoles) was dissolved in 6.8 ml of dichloromethane under a nitrogen atmosphere, 0.63 ml (4.49 mmoles) of triethylamine were added and the reaction solution was then cooled to 0° C. 0.26 ml (2.45 mmoles) of trifluoromethanesulfonyl chloride were subsequently added and the mixture was left under stirring for 48 hours. After LC-MS control, the reaction proved to be complete; the reaction was recovered by adding water to the reaction mixture and then extracting with dichloromethane (3 times). The combined aqueous phases were washed with brine and dried with sodium sulfate. After filtration and evaporation, 510 mg of raw product were obtained which was purified by chromatography on silica (heptane/ethyl acetate gradient 94:6-50:50), isolating 340 mg of the desired product as a yellow oil (yield=47%).
LC-MS [M+H⁺]=356.2.

### EXAMPLE 19

### Preparation of N-(2,6-difluorobenzoyl)-L-proline 3,4,4-triflluorobut-3-en-1-yl ester (Compound Nr. 629)

The substrate (L-proline 3,4,4-trifluorobut-3-en-1-yl ester hydrochloride) (590 mg, 2.27 mmoles) was dissolved in 7.6 ml of dichloromethane under a nitrogen atmosphere, 0.7 ml (5.0 mmoles) of triethylamine were added, and the reaction solution was then cooled to 0° C. 0.34 ml (2.73 mmoles) of 2,6-difluorobenzoyl chloride were subsequently added and the mixture was left under stirring for 16 hours. After LC-MS control, the reaction proved to be complete; the reaction was recovered by adding water to the reaction mixture and then extracting with dichloromethane (3 times). The combined aqueous phases were washed with brine, then dried with sodium sulfate. After filtration and evaporation, 960 mg of raw product was recovered which was purified by chromatography on silica (heptane/ethyl acetate gradient 94:6-50:50), isolating 700 mg of the desired product as a colourless oil (yield=85%).
LC-MS [M+H⁺]=364.1.

### EXAMPLE 20

### Preparation of N-[4-(trifluoromethoxy)-benzoyl]-L-proline 3,4,4-trifluorobut-3-en-1-yl ester (Compound Nr. 630)

The substrate (L-proline 3,4,4-trifluorobut-3-en-1-yl ester hydrochloride) (580 mg, 2.23 mmoles) was dissolved in 7.5 ml of dichloromethane under a nitrogen atmosphere, 0.68 ml (4.91 mmoles) of triethylamine were then added and the reaction solution was then cooled to 0° C. 0.42 ml (2.68 mmoles) of 4-(trifluoromethoxy)benzoyl chloride were subsequently added and the mixture was left under stirring for 48 hours. After LC-MS control, the reaction proved to be complete; the reaction was recovered by adding water to the reaction mixture and then extracting with dichloromethane (3 times). The combined aqueous phases were washed with brine, then dried with sodium sulfate. After filtration and evaporation 1,070 mg of raw product were recovered which was purified by chromatography on silica (heptane/ethyl acetate gradient 92:8-30:70), isolating 890 mg of the desired product as a yellow oil (yield=97%).
LC-MS [M+H⁺]=412.1.

### EXAMPLE 21

### Preparation of N-[2-fluoro-6-(trifluoromethyl)benzoyl]-L-proline 3,4,4-trifluorobut-3-en-1-yl ester (Compound Nr. 631)

The substrate (L-proline 3,4,4-trifluorobut-3-en-1-yl ester hydrochloride) (610 mg, 2.35 mmoles) was dissolved in 7.8 ml of dichloromethane under a nitrogen atmosphere, 0.72 ml (5.17 mmoles) of triethylamine were added and the reaction solution was then cooled to 0° C. 0.44 ml (2.82 mmoles) of 2-fluoro-6-(trifluoromethyl)-benzoyl chloride were subsequently added and the mixture was left under stirring for 16 hours. After LC-MS control, the reaction proved to be complete; the reaction was recovered by adding water to the reaction mixture and then extracting with dichloromethane (3 times). The combined aqueous phases were washed with brine, then dried with sodium sulfate. After filtration and evaporation, 1 g of raw product was recovered which was purified by chromatography on silica (heptane/ethyl acetate gradient 90:10-20:80), isolating 860 mg of the desired product as a colourless oil (yield=89%).
LC-MS [M+H⁺]=414.1.

Operating analogously to what is indicated in the Examples described above, compounds Nr. 1-6, 8-15, 17-20, 22, 26, 35, 64, 522, 562, 619, 620 and 675 indicated in Table 4, were prepared.

TABLE 4

| Comp. | | | | | | | | | | |
|---|---|---|---|---|---|---|---|---|---|---|
| Nr. | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO-Cypropyl | CH₂ | S | F | H | H | — | — | 2 | 0 |
| 8 | N—COCH₃ | CH₃—C—COOCH₃ | S | F | H | H | — | — | 2 | 0 |
| 10 | N—COCH₃ | CH—(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH3)₂ | CH₂ | S | F | H | H | — | — | 2 | 0 |
| 13 | N—CO-Cypentyl | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 22 | N—COCH₃ | CH₂ | S | F | H | H | H | H | 2 | 1 |
| 26 | N—CH₂COCH₃ | CH₂ | S | F | H | H | — | — | 2 | 0 |
| 35 | N—CH₂OCOCH₃ | CH₂ | S | F | H | H | — | — | 2 | 0 |
| 64 | N—CH₂COCH₃ | CF₃—C—CH₃ | O | F | H | H | — | — | 2 | 0 |
| 522 | N—COCH₃ | C═S | S | F | H | H | — | — | 2 | 0 |
| 562 | N—COCH₃ | C═S | O | F | H | H | — | — | 2 | 0 |
| 564 | N—COCH₂CH₃ | C═S | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 675 | N—COCH₃ | C═O | CH₂ | F | H | H | H | H | 2 | 1 |

Table 5 indicates the results of the LC-MS analyses carried out on compounds Nr. 1-6, 8-15, 17-20, 22, 26, 35, 64, 522, 562 and 619, 620 and 675.

TABLE 5

| Comp. Nr. | LC-MS: M+ |
|---|---|
| 1 | 297 |
| 2 | 311 |
| 3 | 315 |
| 4 | 294 |
| 5 | 337 |
| 6 | 309 |
| 8 | 335 |
| 10 | 377 |
| 11 | 325 |
| 12 | 325 |
| 13 | 337 |
| 14 | 351 |
| 15 | 353 |
| 17 | 267 |
| 18 | 335 |
| 19 | 351 |
| 20 | 323 |
| 21 | 343 |
| 22 | 297 |
| 26 | 297 |
| 35 | 313 |
| 64 | 363 |
| 522 | 313 |
| 562 | 297 |
| 564 | 311 |
| 619 | 269 |
| 620 | 297 |
| 675 | 293 |

### EXAMPLE 22

sDetermination of the Nematocidal Activity Against Meloidogyne sp.

The tests aimed at testing the nematicidal activity of the product under examination were carried out using inoculae taken from a farming of Meloidogyne sp. maintained on potted tomato and cucumber plants and grown in greenhouses.

To carry out the experiments, portions of roots having a good number of galls and soil were taken from the infested pots, in which larvae were present starting from the second stage of age.

New pots having a diameter of 15 cm were half filled with sterile soil. The portions of infested roots, previously cleaned, were placed on the same in order to be able to correctly assess the degree of infestation and ensure that each pot contains the same nematic charge. 200-300 g of infested soil were subsequently added, then covered with a thin layer of sterile soil.

The treatment was carried out by pouring 100 ml of solution on the surface of the soil, in which the product to be tested was dissolved.

Tomato or cucumber seedlings at the stage of two or three true leaves were transplanted in the pots thus prepared, one or seven days after application. Different cultivars of tomato or cucumber were used, having a different sensitivity to the parasite and different growth times. In particular, a variety of ornamental tomatoes (cv Microtom), whose seedlings are small in size and are able to reach the ripeness of the fruit in pots and under greenhouse conditions in about two months, was used for assessing the final production.

The containment capacity of the parasite was detected, 30 and 60 days after transplantation, considering the development of the root system (where 100% is the development reached by the healthy comparative root), the fresh weight of the plants treated expressed in grams, and the presence of galls on the roots. The latter was estimated using the infestation scale proposed by Bridge-Page, according to which the value zero corresponds to 0% of the root affected and the value 10 corresponds to 100% of infested root.

Table 6 indicates the results relating to the effectiveness on cucumbers, cv Chinese Long, treating with Compounds Nr. 23, 620, 1, 13, 5, 15, 16, 624 and 625 included in general formula (I) and the reference compounds CR1=Fluensulfone and CR2, described in JP 2000038379, instead of the compound having general formula (I), at a dose of 2000 g/hectare and effecting the survey 30 days after transplantation.

TABLE 6

| | | FRESH WEIGHT OF |
|---|---|---|
| | GALL INDEX | PLANT |
| | (scale 0-10) | (g) |
| COMPOUND | DOSE APPLIED (kg/hectare) | |
| Nr. | 2 | |
| HEALTHY | / | 23 |
| BLANK | | |
| INFESTED | 10 | 3.5 |
| BLANK | | |
| CR1 | 2 | 5.5 |
| CR2 | 2.9 | 17.5 |
| 23 | 2 | 23 |
| 620 | 2.5 | 23 |
| 1 | 3 | 21 |
| 13 | 3 | 18 |
| 5 | 3 | 20 |
| 15 | 2.5 | 18 |
| 16 | 2.9 | 18 |
| 624 | 1.5 | 21 |
| 625 | 2.5 | 22 |

CR1 = 5-chloro-2-(3,4,4-trifluoro-3-butenylsulfonyl)-thiazole, commercial product NIMITZ ® (Fluensulfone)
CR2 = pyridyl-3-carboxylate of 3,4,4-trifluoro-3-butenyl

As can be seen in Table 6, compounds Nr. 23, 15, 16, 620, 624 and 625 have a comparable or greater effectiveness with respect to the reference products. As far as the fresh weight of the plant is concerned, all the compounds in the table gave better results than the reference compounds and in some cases comparable to the healthy blank.

1. A non-aromatic heterocyclic compound with 5 or 6 terms, substituted in position 2 with a fluoroalkenyl chain, having a general formula (I): 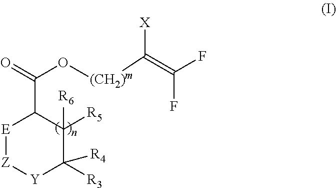
wherein:
E represents an oxygen atom, a sulfur atom or an N-A group;
Y represents a sulfur atom optionally oxidized to group S(O) or S(O)₂, an oxygen atom, an NR₇ group or a CH₂ group;
-A represents a hydrogen atom, a C₁-C₆-alkyl group, a C₁-C₆-haloalkyl group, a C₂-C₇ alkenyl group, a C₂-C₇ haloalkenyl group, a C₃-C₆-cycloalkyl group, a C₃-C₆-cycloalkylalkylC₁-C₆ group, a C₁-C₆-alkylsulfinyl group, a C₁-C₆-alkylsulfonyl group, a C₁-C₆-haloalkylsulfonyl group, an arylsulfonyl group, a C₁-C₆-alkanoyl group, a formyl group, a C₁-C₆-haloalkanoyl group, a C₃-C₆-cycloalkanoyl group, an aroyl group, a heterocyclylcarbonyl group, a C₁-C₆-thioalkanoyl group, a C₁-C₆-thiohaloalkanoyl group, a C₃-C₆-thiocycloalkanoyl group, a thioaroyl group, a thioheterocyclylcarbonyl group, a C₁-C₆-alkoxycarbonyl group, a C₁-C₆-haloalkoxycarbonyl group, a C₃-C₆ cycloalkoxycarbonyl group, an aryloxycarbonyl group, a benzyloxycarbonyl group, a heterocyclyloxycarbonyl group, a C₁-C₆-alkylthiocarbonyl group, a C₁-C₆-haloalkylthiocarbonyl group, a C₃-C₆ cycloalkylthiocarbonyl group, an arylthiocarbonyl group, a benzoylthiocarbonyl group, a heterocyclylthiocarbonyl group, a C₁-C₆-alkylaminocarbonyl group, a C₁-C₆-dialkylaminocarbonyl group, a C₁-C₆-haloalkylaminocarbonyl group, a C₁-C₆-dihaloalkylaminocarbonyl group, a C₃-C₆ cycloalkylaminocarbonyl group, a C₁-C₆-dicycloalkylaminocarbonyl group, an arylaminocarbonyl group, a heterocyclylaminocarbonyl group, a C₁-C₆-alkyl-carbonylalkyl-C₁-C₆ group, a C₁-C₆-haloalkyl-carbonylalkyl-C₁-C₆ group, a C₃-C₆ cycloalkylcarbonylalkyl-C₁-C₆ group, an arylcarbonylalkyl-C₁-C₆ group, a benzylcarbonylalkyl-C₁-C₆ group, a heterocyclycarbonylalkyl-C₁-C₆ group, a C₁-C₆-alkyl-carbonyloxyalkyl-C₁-C₆ group, a C₁-C₆-alkyl-carbonyloxyhaloalkyl-C₁-C₆ group, a C₁-C₆-alkyl-carbonyloxycycloalkyl-C₃-C₆ group, a C₁-C₆-alkyl-carbonyloxyaryl group, a C₁-C₆-alkyl-carbonyloxybenzyl group, a C₁-C₆-alkyl-carbonyloxyheterocyclic group; a C₁-C₆-alkyl-carbonylthioalkyl-C₁-C₆ group, a C₁-C₆-alkyl-carbonylthiohaloalkyl-C₁-C₆ group; a C₁-C₆-alkyl-carbonylthiocycloalkyl-C₃-C₆ group, a C₁-C₆-alkyl-carbonylthioaryl group, a C₁-C₆-alkyl-carbonylthiobenzyl group, a C₁-C₆-alkyl-carbonylthioheterocyclic group, a C₁-C₆-alkyl-aminocarbonylalkyl-C₁-C₆ group, a C₁-C₆-haloalkyl-aminocarbonylalkyl-C₁-C₆ group, a C₁-C₆-dialkyl-aminocarbonylalkyl-C₁-C₆ group, a C₁-C₆-dihaloalkyl-aminocarbonylalkyl-C₁-C₆ group, a C₃-C₆-cycloalkylamino-carbonyl-alkyl-C₁-C₆ group, a C₆-C₁₂-dicycloalkyl-aminocarbonylalkyl-C₁-C₆ group, an aryl-aminocarbonylalkyl-C₁-C₆ group, a benzyl-aminocarbonylalkyl-C₁-C₆ group, a heterocyclyl-aminocarbonylalkyl-C₁-C₆ group, a C₁-C₆-alkanoyloxyalkyl-C₁-C₆ group, a C₁-C₆-haloalkanoyloxyalkyl-C₁-C₆ group, a C₃-C₆-cycloalkanoyloxyalkyl-C₁-C₆ group, an aroyloxyalkyl-C₁-C₆ group, a benzoyloxyalkyl-C₁-C₆ group, a heterocyclylcarbonyloxyalkyl-C₁-C₆ group, a C₁-C₆-alkoxyalkyl-C₁-C₆ group, a C₁-C₆-haloalkoxyalkyl-C1-C₆ group, a C₃-C₆-cycloalkoxyalkyl-C1-C₆ group, an aryloxyalkyl-C₁-C₆ group, a benzyloxyalkyl-C1-C₆ group, a heterocyclyloxyalkyl-C₁-C₆ group, a C₁-C₆-alkanoylthioalkyl-C₁-C₆ group, a C₁-C₆-haloalkanoylthioalkyl-C₁-C₆ group, a C₃-C₆-cycloalkanoylthioalkyl-C1-C₆ group, an aroylthioalkyl-C₁-C₆ group, a benzoylthioalkyl-C₁-C₆ group, a heterocyclylcarbonylthioalkyl-C₁-C₆ group, a C1-C₆-alkylthioalkyl-C1-C₆ group, a C1-C₆-haloalkylthioalkyl-C1-C₆ group, a C₃-C₆-cycloalkylthioalkyl-C₁-C₆ group, an arylthioalkyl-C₁-C₆ group, a benzylthioalkyl-C₁-C₆ group, a heterocyclylthioalkyl-C₁-C₆ group, a C₁-C₆-alkanoylaminoalkyl-C₁-C₆ group, a C₁-C₆-haloalkanoylaminoalkyl-C₁-C₆ group, a C₃-C₆-cycloalkanoylaminoalkyl-C₁-C₆ group, an aroylaminoalkyl-C₁-C₆ group, a benzoylaminoalkyl-C₁-C₆ group, a heterocyclylcarbonylaminoalkyl-C₁-C₆ group, a C₁-C₆-dialkylphosphoryl group, a diarylphosphoryl group, a C₁-C₆-dialkylthiophosphoryl group, a diarylthiophosphoryl group, a 2-oxa-1,3,2-dioxaphosphorinan-2-yl group optionally possibly substituted by one or more C₁-C₆ alkyl groups, a 2-oxa-1,3,2-dioxaphospholan-2-yl group optionally possibly substituted by one or more C₁-C₆ alkyl groups;
Z represents a C═O bond, a C═S bond or a R₁—C—R₂ group: 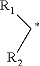
n represents an integer ranging from 0 to 1;
m represents an integer ranging from 1 to 6;
X represents a hydrogen or fluorine atom;
R₁ and R₂ are the same or different and represent a hydrogen atom, a halogen atom selected from fluorine, chlorine, bromine or iodine, a C₁-C₆ alkyl group, a C₁-C₆ haloalkyl group, a C₂-C₇ alkenyl group, a C₂-C₇ haloalkenyl group, a C₃-C₆-cycloalkyl group, an aryl group, a heterocyclic group, a C₃-C₆-cycloalkylalkyl-C₁-C₆ group, a C₁-C₆-alkoxycarbonyl group, a C₁-C₆-haloalkoxycarbonyl group, a C₃-C₆ cycloalkoxycarbonyl group, an aryloxycarbonyl group, a heterocyclyloxycarbonyl group, a C₁-C₆-alkylthiocarbonyl group, a C₁-C₆-haloalkylthiocarbonyl group, a C₃-C₆ cycloalkylthiocarbonyl group, an arylthiocarbonyl group, a heterocyclylthiocarbonyl group, a C₁-C₆-alkylaminocarbonyl group, a C₁-C₆-dialkylaminocarbonyl group, a C₁-C₆-haloalkylaminocarbonyl group, a C₁-C₆-dihaloalkylaminocarbonyl group, a C₃-C₆ cycloalkylaminocarbonyl group, a C₃-C₆ dicycloalkylaminocarbonyl group, an arylaminocarbonyl group, a heterocyclylaminocarbonyl group, a nitrile, a C₁-C₆-alkoxyl group, a C₁-C₆-thioalkoxyl group, a C₁-C₆-haloalkoxyl group, a C₃-C₆-cycloalkoxyl group, a C₁-C₆-halothioalkoxyl group, a C₃-C₆-cyclothioalkoxyl group, a C₁-C₆-alkanoyl group, a C₁-C₆-haloalkanoyl group, a C₃-C₆ cycloalkanoyl group, an aroyl group, a heterocyclylcarbonyl group, a glucosyl group, galactosyl, mannosyl, saccharosyl, lactosyl, maltosyl, arabisonyl, xylosyl, ribosyl, aminoglucosyl, N-acetylaminoglucosyl wherein the OH groups present on said sugars can be optionally substituted to form OR₇ groups;
or R₁ and R₂ together with the carbon atom to which they are bound form a C₃-C₆ cycloalkyl ring;
R₇ represents a C₁-C₆ alkyl group, a benzyl group, a C₁-C₆-alkanoyl group;
R₃, R₄, R₅ and R₆ are the same or different, and represent a hydrogen atom, a halogen atom selected from the group consisting of fluorine, chlorine, bromine or iodine, a C₁-C₆ alkyl group, a C₁-C₆ haloalkyl group or a C₃-C₆ cycloalkyl group, an aryl group, a benzyl group, and a heterocyclic group;
excluding the compound wherein:
E=N-A;
A=benzyloxycarbonyl;
Z═Y═CH₂;
R₃═R₄═H;
n=0;
m=2;
X═F.

2. The compound according to claim 1, wherein the compound having the general formula (I) is selected from compounds wherein E, Z, Y, R₃, R₄, R₅, R₆, X, n and m have the meanings indicated in the following table:

| Comp | | | | | | | | | | |
|---|---|---|---|---|---|---|---|---|---|---|
| Nr | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO-Cypropyl | CH₂ | S | F | H | H | — | — | 2 | 0 |
| 7 | N—COCH₃ | C—(CH₃)₂ | S | F | CH₃ | CH₃ | — | — | 2 | 0 |
| 8 | N—COCH₃ | CH₃—C—COOCH₃ | S | F | H | H | — | — | 2 | 0 |
| 9 | N—COCH₃ | CH- | S | F | H | H | — | — | 2 | 0 |
| | | (1R,2S,3R,4R)-- | | | | | | | | |
| | | (1,2,3,4,5)-(penta | | | | | | | | |
| | | acetoxy)-pentyl | | | | | | | | |
| 10 | N—COCH₃ | CH—(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH₃)₂ | CH₂ | S | F | H | H | — | — | 2 | 0 |
| 13 | N—CO-Cypentyl | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 16 | N—COCH₃ | CF₃—C—CH₃ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 22 | N—COCH₃ | CH₂ | S | F | H | H | H | H | 2 | 1 |
| 23 | N—COCH₃ | CH₂ | S | F | H | H | — | — | 2 | 0 |
| 25 | N—COCH₃ | CH₂ | S(O) | F | H | H | — | — | 2 | 0 |
| 26 | N—CH₂COCH₃ | CH₂ | S | F | H | H | — | — | 2 | 0 |
| 30 | N—H₂CH₂COOCH₃ | CH₂ | S | F | H | H | — | — | 2 | 0 |
| 35 | N—CH₂OCOCH₃ | CH₂ | S | F | H | H | — | — | 2 | 0 |
| 38 | N—H₂OCH(CH₃)₂ | CH₂ | S | F | H | H | — | — | 2 | 0 |
| 39 | N—CH₂OBenzyl | CH₂ | S | F | H | H | — | — | 2 | 0 |
| 44 | N—COCH₃ | CH(CH₃)₂ | S(O)₂ | F | H | H | — | — | 2 | 0 |
| 45 | N—CH₂COCH₃ | CH(CH₃)₂ | S | F | H | H | — | — | 2 | 0 |
| 54 | N—CH₂OCOCH₃ | CH(CH₃)₂ | S | F | H | H | — | — | 2 | 0 |
| 58 | N—CH₂OBenzyl | CH(CH₃)₂ | S | F | H | H | — | — | 2 | 0 |
| 64 | N—CH₂COCH₃ | CF₃—C—CH₃ | O | F | H | H | — | — | 2 | 0 |
| 68 | N—H₂CH₂COOCH₃ | CF₃—C—CH₃ | O | F | H | H | — | — | 2 | 0 |
| 73 | N—CH₂OCOCH₃ | CF₃—C—CH₃ | O | F | H | H | — | — | 2 | 0 |
| 77 | N—CH₂OBenzyl | CF₃—C—CH₃ | O | F | H | H | — | — | 2 | 0 |
| 81 | N—COCH₃ | CH₂ | NH | F | H | H | — | — | 2 | 0 |
| 84 | N—CH₂COCH₃ | CF₃—C—CH₃ | NH | F | H | H | — | — | 2 | 0 |
| 93 | N—CH₂OCOCH₃ | CF₃—C—CH₃ | NH | F | H | H | — | — | 2 | 0 |
| 101 | N—COCH₃ | CH₂ | NCH₃ | F | H | H | — | — | 2 | 0 |
| 104 | N—CH₂COCH₃ | CF₃—C—CH₃ | NCH₃ | F | H | H | — | — | 2 | 0 |
| 113 | N—CH₂OCOCH₃ | CF₃—C—CH₃ | NCH₃ | F | H | H | — | — | 2 | 0 |
| 121 | N—COCH₃ | CH₂ | S | F | H | H | CH₃ | CH₃ | 2 | 1 |
| 123 | N—COCH₃ | CH₂ | S | F | CH₃ | CH₃ | CH₃ | CH₃ | 2 | 1 |
| 126 | N—COCH₃ | CH₂ | S(O)₂ | F | H | H | CH₃ | CH₃ | 2 | 1 |
| 127 | N—COCH₃ | CH₂ | S(O)₂ | F | CH₃ | CH₃ | CH₃ | CH₃ | 2 | 1 |
| 128 | N—COCH₃ | CH₂ | CH₂ | F | H | H | CH₃ | CH₃ | 2 | 1 |
| 129 | N—COCH₃ | CH₂ | CH₂ | F | CH₃ | CH₃ | CH₃ | CH₃ | 2 | 1 |
| 130 | N—COCH₃ | CH₂ | CH₂ | F | H | H | H | H | 2 | 1 |
| 131 | N—COOCH₃ | CH₂ | S | F | H | H | CH₃ | CH₃ | 2 | 1 |
| 132 | N—COOCH₃ | CH₂ | S | F | CH₃ | CH₃ | CH₃ | CH₃ | 2 | 1 |
| 135 | N—COOCH₃ | CH₂ | S(O)₂ | F | H | H | CH₃ | CH₃ | 2 | 1 |
| 136 | N—COOCH₃ | CH₂ | S(O)₂ | F | CH₃ | CH₃ | CH₃ | CH₃ | 2 | 1 |
| 137 | N—COOCH₃ | CH₂ | CH₂ | F | H | H | CH₃ | CH₃ | 2 | 1 |
| 138 | N—COOCH₃ | CH₂ | CH₂ | F | CH₃ | CH₃ | CH₃ | CH₃ | 2 | 1 |
| 139 | N—COOCH₃ | CH₂ | CH₂ | F | H | H | H | H | 2 | 1 |
| 140 | N—COCH₃ | CHCH₃ | S | F | H | H | CH₃ | CH₃ | 2 | 1 |
| 141 | N—COCH₃ | CHCH₃ | S | F | CH₃ | CH₃ | CH₃ | CH₃ | 2 | 1 |
| 144 | N—COCH₃ | CHCH₃ | S(O)₂ | F | H | H | CH₃ | CH₃ | 2 | 1 |
| 145 | N—COCH₃ | CHCH₃ | S(O)₂ | F | CH₃ | CH₃ | CH₃ | CH₃ | 2 | 1 |
| 146 | N—COCH₃ | CHCH₃ | CH₂ | F | H | H | CH₃ | CH₃ | 2 | 1 |
| 147 | N—COCH₃ | CHCH₃ | CH₂ | F | CH₃ | CH₃ | CH₃ | CH₃ | 2 | 1 |
| 148 | N—COCH₃ | CHCH₃ | CH₂ | F | H | H | H | H | 2 | 1 |
| 149 | N—COOCH₃ | CHCH₃ | S | F | H | H | CH₃ | CH₃ | 2 | 1 |
| 150 | N—COOCH₃ | CHCH₃ | S | F | CH₃ | CH₃ | CH₃ | CH₃ | 2 | 1 |
| 153 | N—COOCH₃ | CHCH₃ | S(O)₂ | F | H | H | CH₃ | CH₃ | 2 | 1 |
| 154 | N—COOCH₃ | CHCH₃ | S(O)₂ | F | CH₃ | CH₃ | CH₃ | CH₃ | 2 | 1 |
| 155 | N—COOCH₃ | CHCH₃ | CH₂ | F | H | H | CH₃ | CH₃ | 2 | 1 |
| 156 | N—COOCH₃ | CHCH₃ | CH₂ | F | CH₃ | CH₃ | CH₃ | CH₃ | 2 | 1 |
| 157 | N—COOCH₃ | CHCH₃ | CH₂ | F | H | H | H | H | 2 | 1 |
| 158 | N—COCH₃ | C(CH₃)₂ | S | F | H | H | CH₃ | CH₃ | 2 | 1 |
| 159 | N—COCH₃ | C(CH₃)₂ | S | F | CH₃ | CH₃ | CH₃ | CH₃ | 2 | 1 |
| 162 | N—COCH₃ | C(CH₃)₂ | S(O)₂ | F | H | H | CH₃ | CH₃ | 2 | 1 |
| 163 | N—COCH₃ | C(CH₃)₂ | S(O)₂ | F | CH₃ | CH₃ | CH₃ | CH₃ | 2 | 1 |
| 164 | N—COCH₃ | C(CH₃)₂ | CH₂ | F | H | H | CH₃ | CH₃ | 2 | 1 |
| 165 | N—COCH₃ | C(CH₃)₂ | CH₂ | F | CH₃ | CH₃ | CH₃ | CH₃ | 2 | 1 |
| 166 | N—COCH₃ | C(CH₃)₂ | CH₂ | F | H | H | H | H | 2 | 1 |
| 167 | N—COOCH₃ | C(CH₃)₂ | S | F | H | H | CH₃ | CH₃ | 2 | 1 |
| 168 | N—COOCH₃ | C(CH₃)₂ | S | F | CH₃ | CH₃ | CH₃ | CH₃ | 2 | 1 |
| 172 | N—COOCH₃ | C(CH₃)₂ | CH₂ | F | H | H | CH₃ | CH₃ | 2 | 1 |
| 173 | N—COOCH₃ | C(CH₃)₂ | CH₂ | F | CH₃ | CH₃ | CH₃ | CH₃ | 2 | 1 |
| 174 | N—COOCH₃ | C(CH₃)₂ | CH₂ | F | H | H | H | H | 2 | 1 |
| 178 | COCH₃ | CH—CH₃ | S | F | H | H | — | — | 4 | 0 |
| 179 | N—COCF₃ | CH₂ | S | F | H | H | — | — | 4 | 0 |
| 188 | COCH₃ | CH—CF₃ | S | F | H | H | — | — | 4 | 0 |
| 191 | N—COCH₃ | CH₂ | O | F | H | H | — | — | 4 | 0 |
| 192 | N—COCH₃ | CH—CF₃ | O | F | H | H | — | — | 4 | 0 |
| 196 | N—COCH₃ | CH₂ | S | F | H | H | H | H | 4 | 1 |
| 197 | N—COCH₃ | CH₂ | S | F | H | H | — | — | 4 | 0 |
| 295 | N—COCH₃ | CH₂ | S | F | H | H | CH₃ | CH₃ | 4 | 1 |
| 296 | N—COCH₃ | CH₂ | S | F | CH₃ | CH₃ | CH₃ | CH₃ | 4 | 1 |
| 303 | N—COCH₃ | CH₂ | CH₂ | F | H | H | H | H | 4 | 1 |
| 304 | N—COOCH₃ | CH₂ | S | F | H | H | CH₃ | CH₃ | 4 | 1 |
| 305 | N—COOCH₃ | CH₂ | S | F | CH₃ | CH₃ | CH₃ | CH₃ | 4 | 1 |
| 310 | N—COOCH₃ | CH₂ | CH₂ | F | H | H | CH₃ | CH₃ | 4 | 1 |
| 311 | N—COOCH₃ | CH₂ | CH₂ | F | CH₃ | CH₃ | CH₃ | CH₃ | 4 | 1 |
| 312 | N—COOCH₃ | CH₂ | CH₂ | F | H | H | H | H | 4 | 1 |
| 313 | N—COCH₃ | CHCH₃ | S | F | H | H | CH₃ | CH₃ | 4 | 1 |
| 314 | N—COCH₃ | CHCH₃ | S | F | CH₃ | CH₃ | CH₃ | CH₃ | 4 | 1 |
| 349 | COCH₃ | C═O | S | H | H | H | — | — | 2 | 0 |
| 350 | N—COCH₃ | C—(CH₃)₂ | S | H | H | H | — | — | 2 | 0 |
| 351 | COCH₃ | CH₂ | S(O)₂ | H | H | H | — | — | 2 | 0 |
| 352 | N—COCH₃ | CH—CH₃ | S | H | H | H | — | — | 2 | 0 |
| 353 | COCF₃ | CH₂ | S | H | H | H | — | — | 2 | 0 |
| 355 | N—COCH₃ | C—(CH₃)₂ | S | H | CH₃ | CH₃ | — | — | 2 | 0 |
| 359 | COC(CH₃)₃ | CH₂ | S | H | H | H | — | — | 2 | 0 |
| 362 | N—COCH₃ | CH—CF₃ | S | H | H | H | — | — | 2 | 0 |
| 364 | COCH₃ | CF₃—C—CH₃ | O | H | H | H | — | — | 2 | 0 |
| 365 | N—COCH₃ | CH₂ | O | H | H | H | — | — | 2 | 0 |
| 366 | COCH₃ | CH—CF₃ | O | H | H | H | — | — | 2 | 0 |
| 391 | N—COCH₃ | CH(CH₃)₂ | S(O)₂ | H | H | H | — | — | 2 | 0 |
| 448 | N—COCH₃ | CH₂ | NCH₃ | H | H | H | — | — | 2 | 0 |
| 468 | N—COCH₃ | CH₂ | S | H | H | H | CH₃ | CH₃ | 2 | 1 |
| 469 | N—COCH₃ | CH₂ | S | H | CH₃ | CH₃ | CH₃ | CH₃ | 2 | 1 |
| 472 | N—COCH₃ | CH₂ | S(O)₂ | H | H | H | CH₃ | CH₃ | 2 | 1 |
| 473 | N—COCH₃ | CH₂ | S(O)₂ | H | CH₃ | CH₃ | CH₃ | CH₃ | 2 | 1 |
| 474 | N—COCH₃ | CH₂ | CH₂ | H | H | H | CH₃ | CH₃ | 2 | 1 |
| 475 | N—COCH₃ | CH₂ | CH₂ | H | CH₃ | CH₃ | CH₃ | CH₃ | 2 | 1 |
| 476 | N—COCH₃ | CH₂ | CH₂ | H | H | H | H | H | 2 | 1 |
| 477 | N—COOCH₃ | CH₂ | S | H | H | H | CH₃ | CH₃ | 2 | 1 |
| 490 | N—COCH₃ | CHCH₃ | S(O)₂ | H | H | H | CH₃ | CH₃ | 2 | 1 |
| 491 | N—COCH₃ | CHCH₃ | S(O)₂ | H | CH₃ | CH₃ | CH₃ | CH₃ | 2 | 1 |
| 492 | N—COCH₃ | CHCH₃ | CH₂ | H | H | H | CH₃ | CH₃ | 2 | 1 |
| 493 | N—COCH₃ | CHCH₃ | CH₂ | H | CH₃ | CH₃ | CH₃ | CH₃ | 2 | 1 |
| 494 | N—COCH₃ | CHCH₃ | CH₂ | H | H | H | H | H | 2 | 1 |
| 504 | N—COCH₃ | C(CH₃)₂ | S | H | H | H | CH₃ | CH₃ | 2 | 1 |
| 505 | N—COCH₃ | C(CH₃)₂ | S | H | CH₃ | CH₃ | CH₃ | CH₃ | 2 | 1 |
| 510 | N—COCH₃ | C(CH₃)₂ | CH₂ | H | H | H | CH₃ | CH₃ | 2 | 1 |
| 511 | N—COCH₃ | C(CH₃)₂ | CH₂ | H | CH₃ | CH₃ | CH₃ | CH₃ | 2 | 1 |
| 512 | N—COCH₃ | C(CH₃)₂ | CH₂ | H | H | H | H | H | 2 | 1 |
| 522 | N—COCH₃ | C═S | S | F | H | H | — | — | 2 | 0 |
| 524 | N—COCH₃ | C═S | S(O)₂ | F | H | H | — | — | 2 | 0 |
| 526 | N—COCH₃ | C═O | S(O)₂ | F | H | H | — | — | 2 | 0 |
| 530 | N—COButyl | C═O | S | F | H | H | — | — | 2 | 0 |
| 538 | N—COCH₃ | C═O | S | F | H | H | H | H | 2 | 1 |
| 562 | N—COCH₃ | C═S | O | F | H | H | — | — | 2 | 0 |
| 564 | N—COCH₂CH₃ | C═S | O | F | H | H | — | — | 2 | 0 |
| 568 | N—COCH₃ | C═S | S | F | H | H | H | H | 2 | 1 |
| 588 | N—COCH₃ | C═O | S | H | H | H | H | H | 2 | 1 |
| 589 | N—COCH₃ | C═O | S | H | H | H | CH₃ | CH₃ | 2 | 1 |
| 589 | N—COCH₃ | C═O | S | H | CH₃ | CH₃ | CH₃ | CH₃ | 2 | 1 |
| 593 | N—COCH₃ | C═O | CH₂ | H | H | H | H | H | 2 | 1 |
| 594 | N—COCH₃ | C═O | CH₂ | H | H | H | CH₃ | CH₃ | 2 | 1 |
| 598 | N—COCH₃ | C═S | CH₂ | H | H | H | CH₃ | CH₃ | 2 | 1 |
| 599 | N—COCH₃ | C═S | CH₂ | H | CH₃ | CH₃ | CH₃ | CH₃ | 2 | 1 |
| 600 | N—COCH₃ | CH- | O | F | H | H | — | — | 2 | 0 |
| | | (1R,2S,3R,4R)-- | | | | | | | | |
| | | (1,2,3,4,5)-(penta | | | | | | | | |
| | | acetoxy-pentyl | | | | | | | | |
| 615 | N—CH₃ | CH₂ | O | F | H | H | — | — | 2 | 0 |
| 616 | N—CH(CH₃)₂ | CH₂ | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 621 | N—H | CH₂ | CH₂ | F | H | H | — | — | 2 | 0 |
| 622 | N—COOC(CH₃)₃ | CH₂ | CH₂ | F | H | H | — | — | 2 | 0 |
| 623 | N—CH₃ | CH₂ | CH₂ | F | H | H | — | — | 2 | 0 |
| 624 | N—COCH₃ | CH₂ | CH₂ | F | H | H | — | — | 2 | 0 |
| 625 | N—COCF₃ | CH₂ | CH₂ | F | H | H | — | — | 2 | 0 |
| 626 | N—S(O)₂—CH₃ | CH₂ | CH₂ | F | H | H | — | — | 2 | 0 |
| 627 | N—S(O)₂—CF₃ | CH₂ | CH₂ | F | H | H | — | — | 2 | 0 |
| 628 | N—COButyl | CH₂ | CH₂ | F | H | H | — | — | 2 | 0 |
| 629 | N—CO-(2,4-diF)-Ph | CH₂ | CH₂ | F | H | H | — | — | 2 | 0 |
| 630 | N—CO-(4-OCF₃)—Ph | CH₂ | CH₂ | F | H | H | — | — | 2 | 0 |
| 631 | N—CO-(2-F, 6-CF₃)—Ph | CH₂ | CH₂ | F | H | H | — | — | 2 | 0 |
| 632 | N—COOCH₂CH(CH₃)₂ | CH₂ | CH₂ | F | H | H | — | — | 2 | 0 |
| 633 | N—COOCC(CH₃)₂ | CH₂ | CH₂ | F | H | H | — | — | 2 | 0 |
| 634 | N—COPh | CH₂ | CH₂ | F | H | H | — | — | 2 | 0 |
| 635 | N—CO(4-CF₃)—Ph | CH₂ | CH₂ | F | H | H | — | — | 2 | 0 |
| 636 | N—CO(3-CF₃)—Ph | CH₂ | CH₂ | F | H | H | — | — | 2 | 0 |
| 637 | N—CO-(2,3,6-tri-F, | CH₂ | CH₂ | F | H | H | — | — | 2 | 0 |
| | 5-5-- | | | | | | | | | |
| | OCH₃-)—Ph | | | | | | | | | |
| 638 | N—COEthyl | CH₂ | CH₂ | F | H | H | — | — | 2 | 0 |
| 639 | N—CO-n-Propyl | CH₂ | CH₂ | F | H | H | — | — | 2 | 0 |
| 640 | N—CO-i-Propyl | CH₂ | CH₂ | F | H | H | — | — | 2 | 0 |
| 641 | N—COOCH₃ | CH₂ | CH₂ | F | H | H | — | — | 2 | 0 |
| 642 | N—COO-i-Propyl | CH₂ | CH₂ | F | H | H | — | — | 2 | 0 |
| 643 | N—COO-i-Butyl | CH₂ | CH₂ | F | H | H | — | — | 2 | 0 |
| 644 | N—COOCH₃ | CH₂ | CH₂ | F | H | H | — | — | 2 | 0 |
| 645 | N—P(═O)(OEthyl)₂ | CH₂ | CH₂ | F | H | H | — | — | 2 | 0 |
| 646 | N—P(═O)(OPh)₂ | CH₂ | CH₂ | F | H | H | — | — | 2 | 0 |
| 647 | N—P(═S)(OCH₃)₂ | CH₂ | CH₂ | F | H | H | — | — | 2 | 0 |
| 648 | N-i-propyl | CH₂ | CH₂ | F | H | H | — | — | 2 | 0 |
| 649 | N-t-Butyl | CH₂ | CH₂ | F | H | H | — | — | 2 | 0 |
| 650 | 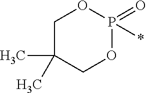 [SMILES: *P1(=O)OCC(C)(C)CO1] | CH₂ | CH₂ | F | H | H | — | — | 2 | 0 |
| 651 | 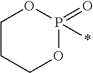 [SMILES: *P1(=O)OCCCO1] | CH₂ | CH₂ | F | H | H | — | — | 2 | 0 |
| 652 | 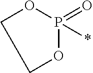 [SMILES: *P1(=O)OCCO1] | CH₂ | CH₂ | F | H | H | — | — | 2 | 0 |
| 675 | N—COCH₃ | C═O | CH₂ | F | H | H | H | H | 2 | 1 |

3. The compound according to claim 1, wherein the compound having the general formula (I) is selected from compounds wherein E, Z, Y, R₃, R₄, R₅, R₆, X, n and m have the meanings indicated in the following table

| Comp. | | | | | | | | | | |
|---|---|---|---|---|---|---|---|---|---|---|
| No | E | Z | Y | X | R₃ | R₄ | R₅ | R₆ | m | n |
| 1 | N—COCH₃ | C═O | S | F | H | H | — | — | 2 | 0 |
| 2 | N—COCH₃ | C—(CH₃)₂ | S | F | H | H | — | — | 2 | 0 |
| 3 | N—COCH₃ | CH₂ | S(O)₂ | F | H | H | — | — | 2 | 0 |
| 4 | N—COCH₃ | CH—CH₃ | S | F | H | H | — | — | 2 | 0 |
| 5 | N—COCF₃ | CH₂ | S | F | H | H | — | — | 2 | 0 |
| 6 | N—CO(Cypropyl) | CH₂ | S | F | H | H | — | — | 2 | 0 |
| 7 | N—COCH₃ | C—(CH₃)₂ | S | F | CH₃ | CH₃ | — | — | 2 | 0 |
| 8 | N—COCH₃ | CH₃—C—COOCH₃ | S | F | H | H | — | — | 2 | 0 |
| 9 | N—COCH₃ | CH-(1R,2S,3R,4R)-- | S | F | H | H | — | — | 2 | 0 |
| | | (1,2,3,4,5)-(penta | | | | | | | | |
| | | acetoxy)-pentyl | | | | | | | | |
| 10 | N—COCH₃ | CH—(4F)-phenyl | S | F | H | H | — | — | 2 | 0 |
| 11 | N—COC(CH₃)₃ | CH₂ | S | F | H | H | — | — | 2 | 0 |
| 12 | N—COCH₂CH(CH₃)₂ | CH₂ | S | F | H | H | — | — | 2 | 0 |
| 13 | N—CO(Cypentyl) | CH₂ | S | F | H | H | — | — | 2 | 0 |
| 14 | N—COCH₃ | CH—CF₃ | S | F | H | H | — | — | 2 | 0 |
| 15 | N—COOC(CH₃)₃ | C—(CH₃)₂ | O | F | H | H | — | — | 2 | 0 |
| 16 | N—COCH₃ | CF₃—C—CH₃ | O | F | H | H | — | — | 2 | 0 |
| 17 | N—COCH₃ | CH₂ | O | F | H | H | — | — | 2 | 0 |
| 18 | N—COCH₃ | CH—CF₃ | O | F | H | H | — | — | 2 | 0 |
| 19 | N—COCH₃ | CH-Cypentyl | S | F | H | H | — | — | 2 | 0 |
| 20 | N—COCH₃ | CH-Cypropyl | S | F | H | H | — | — | 2 | 0 |
| 22 | N—COCH₃ | CH₂ | S | F | H | H | H | H | 2 | 1 |
| 23 | N—COCH₃ | CH₂ | S | F | H | H | — | — | 2 | 0 |
| 26 | N—CH₂COCH₃ | CH₂ | S | F | H | H | — | — | 2 | 0 |
| 35 | N—CH₂OCOCH₃ | CH₂ | S | F | H | H | — | — | 2 | 0 |
| 64 | N—CH₂COCH₃ | CF₃—C—CH₃ | O | F | H | H | — | — | 2 | 0 |
| 522 | N—COCH₃ | C═S | S | F | H | H | — | — | 2 | 0 |
| 562 | N—COCH₃ | C═S | O | F | H | H | — | — | 2 | 0 |
| 564 | N—COCH₂CH₃ | C═S | O | F | H | H | — | — | 2 | 0 |
| 619 | N—COH | CH₂ | S | F | H | H | — | — | 2 | 0 |
| 620 | N—COCH₂CH₃ | CH₂ | S | F | H | H | — | — | 2 | 0 |
| 621 | N—H | CH₂ | CH₂ | F | H | H | — | — | 2 | 0 |
| 622 | N—COOC(CH₃)₃ | CH₂ | CH₂ | F | H | H | — | — | 2 | 0 |
| 623 | N—CH₃ | CH₂ | CH₂ | F | H | H | — | — | 2 | 0 |
| 624 | N—COCH₃ | CH₂ | CH₂ | F | H | H | — | — | 2 | 0 |
| 625 | N—COCF₃ | CH₂ | CH₂ | F | H | H | — | — | 2 | 0 |
| 626 | N—S(O)₂—CH₃ | CH₂ | CH₂ | F | H | H | — | — | 2 | 0 |
| 627 | N—S(O)₂—CF₃ | CH₂ | CH₂ | F | H | H | — | — | 2 | 0 |
| 629 | N—CO—(2,4-diF)—Ph | CH₂ | CH₂ | F | H | H | — | — | 2 | 0 |
| 630 | N—CO—(4-OCF₃)—Ph | CH₂ | CH₂ | F | H | H | — | — | 2 | 0 |
| 631 | N—CO—(2-F,6-CF₃)—Ph | CH₂ | CH₂ | F | H | H | — | — | 2 | 0 |
| 675 | N—COCH₃ | C═O | CH₂ | F | H | H | H | H | 2 | 1 |

wherein Cy represents the abbreviation of cyclo and Ph represents the abbreviation of phenyl.

4. Use of the compound according to claim 1, for the control of nematodes in agricultural crops.

5. Use according to claim 4 for the control of nematodes selected from the group consisting of Pratylenchus spp., Globodera spp., Heterodera spp., Meloidogyne spp., Aphelenchoides spp., Radopholus similis, Ditylenchus dipsaci, Tylenchulus semipenetrans, Longidorus spp., Xiphinema spp., Trichodorus spp., Bursaphelenchus spp., and Belonolaimus spp., etc., preferably by application before transplanting/sowing or during the growth of the plant, via the leaves, on the ground by fertigation, incorporation in the soil, and/or by seed treatment, of one or both a preventive and curative nature.

6. The nematocidal agronomic composition comprising one or more compounds having the formula (I) according to claim 1, a solvent and/or solid, liquid or liquefied diluent, and optionally one or more surfactants and/or other agronomically acceptable co-formulants.

7. The composition according to claim 6, comprising at least one compound having the formula (I) and at least one second active ingredient selected from the group consisting of insecticides, acaricides, nematocides different from those having formula (I), herbicides, fungicides, bactericides, fertilizers and biostimulants.

8. Use of the composition according to claim 6, for the control of nematodes in agricultural crops.

9. Use according to claim 8, for the control of nematodes selected from the group consisting of Pratylenchus spp, Globodera spp, Heterodera spp, Meloidogyne spp, Aphelenchoides spp, Radopholus similis, Ditylenchus dipsaci, Tylenchulus semipenetrans, Longidorus spp, Bursaphelenchus spp, and Belonolaimus spp., by application before transplanting/sowing or during the growth of the plant, via the leaves, on the ground by fertigation, incorporation in the soil, and/or by seed treatment, of one or both a preventive and curative nature.

10. A method for the control of nematodes in cultivated areas, consisting of applying effective and non-phytotoxic doses of a composition comprising at least one compound having the formula (I) according to claim 1, and, optionally, one or more known active ingredients compatible therewith, on any part of the plant to be protected.
